SEQUENCE LISTING (228 sequences)

SEQ ID NO: 1 - DNA, 1053 bp, Arabidopsis thaliana
atgtggacgatgggttacaacgaaggaggcgcagattccttcaacggaggaagaaagcttcgtcctctcatcccacgtctcacttcttgccccaccgccgccgtaaacaccaactccgaccaccgctttaatatggcagtggtgacgatgacggcggagcagaacaagagggagctgatgatgctaaactcagaacctcaacatccaccggtaatggtgagctcacggtggaatccgacaccagatcagttaagggttcttgaagagctttaccgacaaggaactagaactccttctgccgaccacatccaacaaatcaccgcacagctacgacggtacgggaagatagaaggcaaaaacgttttctattggttccaaaaccacaaagcccgagaacgccagaaacgacggcgacaaatggaaactggccacgaagaaacggtcctttcaacagcaagtcttgtctcaaaccacggattcgacaagaaagatcctccaggttacaaggttgaacaggtcaagaactggatatgttcggtcggatgcgacacgcaaccagagaaaccttcccgtgattatcatctggaggagccagcgaatatacgggtggagcacaatgctcgttgtggaggagacgagagacgaagctttttagggataaacaccacgtggcagatgatgcagttgccacctagtttctactcttcttcacatcatcatcatcaacgtaacctcattctcaattcacctactgtttcctccaacatgtccaacagcaacaatgctgtctctgcttctaaagatacggtcacggtttcacctgtatttctacgtacgagagaagcaacgaacacagagacttgtcatcgaaatggtgatgataataaagatcaagaacaacatgaagattgctccaacggtgaattggatcatcaagaacagacacttgagctgtttccactgagaaaagaagggttttgtagcgatggtgagaaagacaagaacattagtggcattcactgtttctatgagtttctgccattgaagaactaa

SEQ ID NO: 2 - protein, 350 aa, Arabidopsis thaliana
MWTMGYNEGGADSFNGGRKLRPLIPRLTSCPTAAVNTNSDHRFNMAVVTMTAEQNKRELMMLNSEPQHPPVMVSSRWNPTPDQLRVLEELYRQGTRTPSADHIQQITAQLRRYGKIEGKNVFYWFQNHKARERQKRRRQMETGHEETVLSTASLVSNHGFDKKDPPGYKVEQVKNWICSVGCDTQPEKPSRDYHLEEPANIRVEHNARCGGDERRSFLGINTTWQMMQLPPSFYSSSHHHHQRNLILNSPTVSSNMSNSNNAVSASKDTVTVSPVFLRTREATNTETCHRNGDDNKDQEQHEDCSNGELDHQEQTLELFPLRKEGFCSDGEKDKNISGIHCFYEFLPLKN

SEQ ID NO: 3 - DNA, 1062 bp, Brassica rapa
atgtggatgatgggttacaacgaaggaggtgcagattctttcaacggaggaagaaagcttcgtcctctcatccctcgcctctcttcttgccccaccgccaccggaaacaccaactccgaccaccgctttaatatggcagtggtgacaacaatgacggcggagcagaacaagagggagttgatgatgctaaactcagagcctcaacatccaccagcaatggtgagctcacggtggaatccgacaccggatcagttaaaggctcttgaagagctttaccaacaaggaacaagaactccttcggccgaccatatccaacaaatcaccgcgcagctacgacggtacgggaagatagaaggcaaaaatgttttctattggttccaaaaccacaaagctcgcgaacgccagaagcgtcgacgacaaatggaaaatggccacgatgaatcagtcgttacgacaacgagtcttgtctcaaaccaaggattcgacaagaaagattcaaaaggttacaaggttgaacataccaagaactggatatgttcggtcggatgcgacacacaacaagagaaacctcatgaccatcatcatcaggaggagcctgtctcaatagcgatggtagcaacgaaacgcaatggtcgtcatggaggagacgagatacaaagctttcctgtgagaaatgccaggtggcatatgatgcagctaccacctggactctacccttcttcacaacaacagcataataatcaccaactcaatctcaattccactacagtttctttaaacttgtccactagcaacagtaacgttcccgctgctaaagatacagtcacattttcaccttcatttctgaggacaagaggagcaaggaacacagaggcttgtgatgataataaagatcaagaacaacatgaagattgctcaaacggtgaattggaacatcaagaacagacactagagctgtttccattgagaaaagaggggttttgtagcaatggtggcgatgatgggaaagagaaggagagtggcattcaatgtttctacgagtttcttccattgaagaactag

SEQ ID NO: 4 - protein, 353 aa, Brassica rapa
MWMMGYNEGGADSFNGGRKLRPLIPRLSSCPTATGNTNSDHRFNMAVVTTMTAEQNKRELMMLNSEPQHPPAMVSSRWNPTPDQLKALEELYQQGTRTPSADHIQQITAQLRRYGKIEGKNVFYWFQNHKARERQKRRRQMENGHDESVVTTTSLVSNQGFDKKDSKGYKVEHTKNWICSVGCDTQQEKPHDHHHQEEPVSIAMVATKRNGRHGGDEIQSFPVRNARWHMMQLPPGLYPSSQQQHNNHQLNLNSTTVSLNLSTSNSNVPAAKDTVTFSPSFLRTRGARNTEACDDNKDQEQHEDCSNGELEHQEQTLELFPLRKEGFCSNGGDDGKEKESGIQCFYEFLPLKN

SEQ ID NO: 5 - DNA, 1056 bp, Brassica rapa
atgtggatgatgggttacaacgaaggaggtgcagattccttcaacggaggaagaaaacttcgccctctcatcccacgcctctcctcttgccccagtgccgccgcaaacaccaactccgaccaccgctttaacatggtgatggctacaatgacggctgagcagaacaaaagggagctcatgatgctaaactcagaacatcaacatccacctatgatggtgagctcacgatggaaccccacaccggatcagttaaaggctcttgaagagctttaccaacaaggaactagaactccttctgccgaccatatccaacaaatcaccgcgcagctacgtaggtacggaaagatcgaaggcaaaaatgttttctattggttccaaaaccacaaagcacgtgaacgccaaaaacggcgacgacaaatggaaaatggccacgacgaatctgtctttacgacaactagtcttgtctcaaatcacggtttcgacaagaaatatccaccaggttacaaggttgaacagacgaagaaatggatatgttcggtcggatgcgacacacaaccacagaaacctcatgaacttcatcatcaggaagagcctgtctccatagcattaccaacaaagggcaatggtcgttatgtaggaaacgagagacaaagctttcccgggagaaatgccatgtcgcagatgatgcaacttgcacctggacattacccttattcacgacaacaacaacatcatcatgaactcattctcaattcaactactgttacgtctagcttctctgctagcaacagtactgtttccgctgctaaagataccgtcacagttacacctttatttctgaggacaagagcagcaaccaacacaggaacttgtgatgataataaagatcaagaacgtcatgaagattgcgtaaacggtgaattggatcaccaagaaaagacactagagctgtttccactgagaaaagaaggattttgtatcagtggtggagatgatgggaaagagaaagagagtggcattcactgtttctatgagtttcttccattgaagaactaa

SEQ ID NO: 6 - protein, 351 aa, Brassica rapa
MWMMGYNEGGADSFNGGRKLRPLIPRLSSCPSAAANTNSDHRFNMVMATMTAEQNKRELMMLNSEHQHPPMMVSSRWNPTPDQLKALEELYQQGTRTPSADHIQQITAQLRRYGKIEGKNVFYWFQNHKARERQKRRRQMENGHDESVFTTTSLVSNHGFDKKYPPGYKVEQTKKWICSVGCDTQPQKPHELHHQEEPVSIALPTKGNGRYVGNERQSFPGRNAMSQMMQLAPGHYPYSRQQQHHHELILNSTTVTSSFSASNSTVSAAKDTVTVTPLFLRTRAATNTGTCDDNKDQERHEDCVNGELDHQEKTLELFPLRKEGFCISGGDDGKEKESGIHCFYEFLPLKN

SEQ ID NO: 7 - DNA, 492 bp, Medicago truncatula
atgtggatggtgggttacaatgaaggtggtgagttcaacatggctgattatccattctgtggaaggaaactaaggcctctcatgccaagaccagtcctagtccctactacttctcctaacaacacttcaactataactccttccttaaaccgcattcatggtggcaatgatttattttcacaatatcatcacaatctgcggcagcaagcatcagtaggagatcatagcaagagatcagagttgaataataataataattcatctgcaacagtggaagaattgtatagaagaggaacaagaacaccttatgctgagcaaatccaacagatcactgcccagcttagaaaatttggaaaaattgaaggcaaaaatgttttctattggtttcagaatcacaaagccagagaaaggcaaaaacgacgccgtcaaatgttcttagtgaaaatgggcttttggagagtgttgttttcggttggttttggagagaaatga

SEQ ID NO: 8 - protein, 163 aa, Medicago truncatula
MWMVGYNEGGEFNMADYPFCGRKLRPLMPRPVLVPTTSPNNTSTITPSLNRIHGGNDLFSQYHHNLRQQASVGDHSKRSELNNNNNSSATVEELYRRGTRTPYAEQIQQITAQLRKFGKIEGKNVFYWFQNHKARERQKRRRQMFLVKMGFWRVLFSVGFGEK

SEQ ID NO: 9 - DNA, 555 bp, Medicago truncatula
atgtctgattcattctgtagctttctctctgccacttcaaattccaaccataattcccatgcaccttcaaaaatcacctcaatagtccaaccttattgtatttgcacccattgcaaccatctcctccccttcaatcaccatctcgctgagcaaggaacaagcaatattatgcatcctcaaccaagctcaaggtggagtccaacaccagttcaattactagtccttgaagaattgtataggcaaggcatgaaaacaccatcagctgaacaaatccaacaaattgcttcgcagcttcgacaattcgggaagattgaaggaaaaaatgtgttctattggttccagaatcacaaggcgagagagcgacagaagcgtcgtcgccttgagatggaggaaacaactgaagataaaaaagagaaagagaagtacgttatgggaaattcaaaaaagcaagaaggtgcagagacaggtggtggagttaaagagacaaagaagtgggcaaccacttcaaactgcagtgaacaagcagaggtctgtaatttctcttgtctttgttga

SEQ ID NO: 10 - protein, 184 aa, Medicago truncatula
MSDSFCSFLSATSNSNHNSHAPSKITSIVQPYCICTHCNHLLPFNHHLAEQGTSNIMHPQPSSRWSPTPVQLLVLEELYRQGMKTPSAEQIQQIASQLRQFGKIEGKNVFYWFQNHKARERQKRRRLEMEETTEDKKEKEKYVMGNSKKQEGAETGGGVKETKKWATTSNCSEQAEVCNFSCLC

SEQ ID NO: 11 - DNA, 498 bp, Medicago truncatula
atgtggatggtgggttacagtgaaggtggtgagttcaacatggctgattatccattatgtggaaggaaactaaggcctctcatgtcaagaccagtcccaatcccagtccctactacttctcctaacaacacttcaactataactccttccttaaaccgcattcatggtggcaatgatttattttcacaatatcatcacaatctgcagcagcaagcatcagtaggagatcatagcaagagatcagagttgaataataataataatccatctgcagcagtggaagaattgtatagaagaggaacaagaacaccttctgctgagcaaatccaacaaatcactgcccagcttagaaaaattggaaaaattgaaggcaaaaatgttttctattggtttcagaatcacaaagccagagaaaggcaaaaacgacgccgtcaaatgttcttagtgaaaatgggcttttggagagtgttgttttcggttggttttggagagaaatga

SEQ ID NO: 12 - protein, 165 aa, Medicago truncatula
MWMVGYSEGGEFNMADYPLCGRKLRPLMSRPVPIPVPTTSPNNTSTITPSLNRIHGGNDLFSQYHHNLQQQASVGDHSKRSELNNNNNPSAAVEELYRRGTRTPSAEQIQQITAQLRKIGKIEGKNVFYWFQNHKARERQKRRRQMFLVKMGFWRVLFSVGFGEK

SEQ ID NO: 13 - DNA, 1077 bp, Medicago truncatula
atgtggatggtgggttacaatgaaggtggtgagttcaacatggctgattatccattcagtggaaggaaactaaggcctctcattccaagaccagtcccagtccctactacttctcctaacaacacttcaactataactccttccttaaaccgcattcatggtggcaatgatttattttcacagtatcatcataatctgcagcagcaagcatcagtaggagatcatagcaagagatcagagttgaataataataataatccatctgcagcagttgtggtgagttcaagatggaatccaacaccagaacagttaagagcactggaagaattgtatagaagaggaacaagaacaccttctgctgagcaaatccaacaaataactgcccagcttagaaaatttggaaaaattgaaggcaaaaatgttttctattggtttcagaatcacaaagcaagagaaaggcaaaaacgacggcgtcaaatggaatcagcggctgctgagtttgattctgctcttgaaaagaaagacttaggcgcaagtaggacagtgtttgaagttgaacacactaaaaactggctaccatctacaaattccagtaccagtactcttcatcttgcagaggaatctgtttcaattcaaaggtcagcagcagcagcaaaagcagatggatggctccaattcgatgaagcagaattacagcaaagaagaaactttatggaaaggaatgccacgtggcatatgatgcagttaacttcttcttgtcctacagctagcatgtccaccacaaccacagtaacaactagacttatggacccaaaactcatcaagacccatgaactcaacttattcatttcacctcacacatacaaagaaagagaaaacgcttttatccacttaaatactagtagtactcatcaaaatgaatctgatcaaacccttcaacttttcccaataaggaatggagatcatggatgcactgatcatcatcatcatcataacattatcaaagagacacagatatcagcttcagcaatcaatgcacccaaccagtttattgagtttcttcccttgaaaaactga

SEQ ID NO: 14 - protein, 358 aa, Medicago truncatula
MWMVGYNEGGEFNMADYPFSGRKLRPLIPRPVPVPTTSPNNTSTITPSLNRIHGGNDLFSQYHHNLQQQASVGDHSKRSELNNNNNPSAAVVVSSRWNPTPEQLRALEELYRRGTRTPSAEQIQQITAQLRKFGKIEGKNVFYWFQNHKARERQKRRRQMESAAAEFDSALEKKDLGASRTVFEVEHTKNWLPSTNSSTSTLHLAEESVSIQRSAAAAKADGWLQFDEAELQQRRNFMERNATWHMMQLTSSCPTASMSTTTTVTTRLMDPKLIKTHELNLFISPHTYKERENAFIHLNTSSTHQNESDQTLQLFPIRNGDHGCTDHHHHHNIIKETQISASAINAPNQFIEFLPLKN

SEQ ID NO: 15 - DNA, 918 bp, Phseolus vulgaris
atgtcggattcatttgctagtacctttcactcttccactgcaaacatcacccacaattcccatgcaacccccaaaatcaccacattatgctcgatagcccaaccttattgtatttgcactcactgcaaccacatcctcgccttcaaccagcatgttggtaatgtagcagaggaagggacaaataacacagggtcccatgtgcagtcccagcaaagcacaaggtggagtccaacgccagttcagttactagtccttgaggaattgtatagacaaggcactaaaacaccatcagctgaacagattcaacaaatagcttcacagttgcgccagtttgggaagattgaagggaagaacgtgttctactggttccagaatcacaaggcgagagagagacaaaagaggcgccgcagggagatggaggaaaataatgctgcttcttctagtgaagggttgaaagagacaggttgtggagttaaagagacaaagaagtgggcatccacttcaaactgcagtcgacatgcagaggaatctgctgcactggatatagcagaaaagggctctcatgggtgcactcagtttgaggagagaggcatacaagttttgaggagaaacatagcagaaaggaagttgcaattgcaagataacatggaaatgccttgtttccctcccactactatggcagctcttgcaacttcacacagaacacttaacgcacaacttagttacaacagagaaagtttaaactgctatgatggagaaaatgcagatcctcgaacccttgatctatttccacataagagggatgaccaagatgacatttgtcttgctgagaggaaatctatgttctgtgccagtgcttctatggacgctgatatcacctcaagccagtattttgagtttcttcctttaagaaactga

SEQ ID NO: 16 - protein, 305 aa, Phseolus vulgaris
MSDSFASTFHSSTANITHNSHATPKITTLCSIAQPYCICTHCNHILAFNQHVGNVAEEGTNNTGSHVQSQQSTRWSPTPVQLLVLEELYRQGTKTPSAEQIQQIASQLRQFGKIEGKNVFYWFQNHKARERQKRRRREMEENNAASSSEGLKETGCGVKETKKWASTSNCSRHAEESAALDIAEKGSHGCTQFEERGIQVLRRNIAERKLQLQDNMEMPCFPPTTMAALATSHRTLNAQLSYNRESLNCYDGENADPRTLDLFPHKRDDQDDICLAERKSMFCASASMDADITSSQYFEFLPLRN

SEQ ID NO: 17 - DNA, 1080 bp, Phseolus vulgaris
atgtggatggttggttacaacgaaggtggtgagttcagcatggctgattatggactcaatggaagaaaactcaggcctctcatgccaaggccagtcacttctcccaacaacactccaaacacaaactctccatgcttgagccgcatccatcatggcaacgactttttttcacaatatcacaatctggcatcagtggcggatcagggcaagagagagttcaatcctccacccgtagtggtgagttcaaggtggaatccaactccggagcagctaagggcattggaggaattgtacagaagagggacaagaacaccatctgccgagcaaatccaacaaatcacggcacagcttcgaaggtttggaaaaattgaagggaagaatgttttctattggtttcagaatcacaaagcgagggaaagacagaaacgccgccgtcaaatggagtcagcggctgagggtcatcacacccgtgactttgatggtactcttgacaagaaagacttaggggcaagtaggacagtgtttgaagtggaacagaccaagaactgggaatcctctacaaactgcagtacccttgcaaaggaatctgtttcaatacaaagggcagcagcaaaagcagattgtagaacagaaggatggctccaattcgatgaagaagagttacaccacacacagcacacaagaaacttaatggagaggaatgccacgtggcacatgatgcagttatcttgtcctccccctccttcggtttcaccccacctcataaacacacctcctaccatctctacttctagcatggccaccacaccctctgtcacagcaagagtaatggacccaaagctcattaagactcatgatctcagcttcttcatttcacctaaccgagaaaacggtattatccacttaagcagtatcaacaccgatgataattctgtggaatctcaaacacttcaacttttcccaataaggaacggggatgaaagcagtgacaaccttaaccaccacaaagagacagaggtatcggtctcagcaatgaatgcccccagccagttttttgagttccttccattgaagaagtga

SEQ ID NO: 18 - protein, 359 aa, Phseolus vulgaris
MWMVGYNEGGEFSMADYGLNGRKLRPLMPRPVTSPNNTPNTNSPCLSRIHHGNDFFSQYHNLASVADQGKREFNPPPVVVSSRWNPTPEQLRALEELYRRGTRTPSAEQIQQITAQLRRFGKIEGKNVFYWFQNHKARERQKRRRQMESAAEGHHTRDFDGTLDKKDLGASRTVFEVEQTKNWESSTNCSTLAKESVSIQRAAAKADCRTEGWLQFDEEELHHTQHTRNLMERNATWHMMQLSCPPPPSVSPHLINTPPTISTSSMATTPSVTARVMDPKLIKTHDLSFFISPNRENGIIHLSSINTDDNSVESQTLQLFPIRNGDESSDNLNHHKETEVSVSAMNAPSQFFEFLPLKK

SEQ ID NO: 19 - DNA, 1071 bp, Phseolus vulgaris
atgtggatggtgggtgacaatgaaggtggtgacttcaacatggttgatcattctttcagtggaaggaaactcatgcctctcatgccaaggtcaatcacttctctcaacactgcaccaccaacaaccactcttagcttaactcaccctcatggcaatcatttcccttcacagtatgattatcaccatcttgaacaaaacaagagagagcagttcaatggtacagcacccgctgtggtgagttcaaggtggaatccaacccgagagcaactaagagcccttgaagagttgtatagaagaggaacaaggacaccatctgctgagcaaatccaacatatcactgcacaacttagaagatttggtaacatagaagggaagaatgtgttctattggtttcagaatcacaaagccagggagaggcaaaagcgtcgccgccaaatggaatcggatgccgaaactcctgagaagaaagattcagctgcaagtaggaccgagtttgaagttaaacagacaaaaaattggacaccttcaacaaactgcagtactattgcagaggaatctgtttcaacacagaggactgcaaaagcagaggctgtagagagtagaacagttggatggctccaattcgatgaaggggaattccaacaaagaagaaaccttttggagaggaatgcaacgtggcatgtgatgcagttaccttgtccttctcctgctgcacctgtcacacacctcataaacagtcctcctaatgcctcttcagctattactatggccactacaacaagcacagtaacaacaagaaaaatggacccaaacctcattaagacacatgatctcagcttttttatttcaccccagagggaaaacagtgttatctacttaagcagcagcagtagtactactgaagatgataactgtgtggagtctcaaacccttcaatttttcccattaaggagtggtgatgctagcagtgacaacatgagggataaagagacagagatatcagcttcagcaatgaattgcagtaacttgaccccaagccagttttttgagttccttcctatgaaggagtaa

SEQ ID NO: 20 - protein, 356 aa, Phseolus vulgaris
MWMVGDNEGGDFNMVDHSFSGRKLMPLMPRSITSLNTAPPTTTLSLTHPHGNHFPSQYDYHHLEQNKREQFNGTAPAVVSSRWNPTREQLRALEELYRRGTRTPSAEQIQHITAQLRRFGNIEGKNVFYWFQNHKARERQKRRRQMESDAETPEKKDSAASRTEFEVKQTKNWTPSTNCSTIAEESVSTQRTAKAEAVESRTVGWLQFDEGEFQQRRNLLERNATWHVMQLPCPSPAAPVTHLINSPPNASSAITMATTTSTVTTRKMDPNLIKTHDLSFFISPQRENSVIYLSSSSSTTEDDNCVESQTLQFFPLRSGDASSDNMRDKETEISASAMNCSNLTPSQFFEFLPMKE

SEQ ID NO: 21 - protein, 221 aa, Zea mays
WATKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMAPPPPAACNDNGGARVIYRNPFYVAAPQAPPANAAYYYPQPQQQQQQQVTVMYQYPRMEVAGQDKMMTRAAAHQQQQHNGAGQQPGRAGHPSRETLQLFPPPAHLRAAARQGARRQRQ

SEQ ID NO: 22 - DNA, 783 bp, Arabidopsis thaliana
atggaaaacgaagtaaacgcaggaacagcaagcagttcaagatggaacccaacgaaagatcagatcacgctactggaaaatctttacaaggaaggaatacgaactccgagcgccgatcagattcagcagatcaccggtaggcttcgtgcgtacggccatatcgaaggtaaaaacgtcttttactggttccagaaccataaggctaggcaacgccaaaagcagaaacaggagcgcatggcttacttcaatcgcctcctccacaaaacctcccgtttcttctacccccctccttgctcaaacgtgggttgtgtcagtccgtactatttacagcaagcaagtgatcatcatatgaatcaacatggaagtgtatacacaaacgatcttcttcacagaaacaatgtgatgattccaagtggtggctacgagaaacggacagtcacacaacatcagaaacaactttcagacataagaacaacagcagccacaagaatgccaatttctccgagttcactcagatttgacagatttgccctccgtgataactgttatgccggtgaggacattaacgtcaattccagtggacggaaaacactccctctttttcctcttcagcctttgaatgcaagtaatgctgatggtatgggaagttccagttttgcccttggtagtgattctccggtggattgttctagcgatggagccggccgagagcagccgtttattgatttcttttctggtggttctacttctactcgtttcgatagtaatggtaatgggttgtaa

SEQ ID NO: 23 - protein, 260 aa, Arabidopsis thaliana
MENEVNAGTASSSRWNPTKDQITLLENLYKEGIRTPSADQIQQITGRLRAYGHIEGKNVFYWFQNHKARQRQKQKQERMAYFNRLLHKTSRFFYPPPCSNVGCVSPYYLQQASDHHMNQHGSVYTNDLLHRNNVMIPSGGYEKRTVTQHQKQLSDIRTTAATRMPISPSSLRFDRFALRDNCYAGEDINVNSSGRKTLPLFPLQPLNASNADGMGSSSFALGSDSPVDCSSDGAGREQPFIDFFSGGSTSTRFDSNGNGL

SEQ ID NO: 24 - DNA, 633 bp, Brassica rapa
atggaaaacgaagggagcgcaggaacgggaagcagtcctcgttggaacccaacgaaagaacagataacgttgcttgagaatctttacaagcaggggatacgaacaccgagggccgatcagatacagcagatcaccggtaggcttcgagcacacggccatatcgaggtgggctgcgttaatccgtactatttacatcaagtaagagatcatcataatcaacatggaagtgtatacagacaagatcttcacagtaacaaagtaatgcttccaagtggtggctacgagaaacgcactaccacagatcataagaaacatgattcagacataacaacaacaacagctagaatgtcaatgtcttcgagttcattcagatttgaccgatttgcccttggtaatcacggttattttagcgagggcatcaacgtcaataccaatggactgaagacgcttccgctttttccacttcagcctctggatgctgctaactactctggtgttggaaattccagtttttcgcttcgtcatgattctccggtgacttattgtggcgatgatggcggacgagggcagccgtttattgacttcttctctagtggttctagtaggttcaataatagtggaaacgggttgtaa

SEQ ID NO: 25 - protein, 210 aa, Brassica rapa
MENEGSAGTGSSPRWNPTKEQITLLENLYKQGIRTPRADQIQQITGRLRAHGHIEVGCVNPYYLHQVRDHHNQHGSVYRQDLHSNKVMLPSGGYEKRTTTDHKKHDSDITTTTARMSMSSSSFRFDRFALGNHGYFSEGINVNTNGLKTLPLFPLQPLDAANYSGVGNSSFSLRHDSPVTYCGDDGGRGQPFIDFFSSGSSRFNNSGNGL

SEQ ID NO: 26 - DNA, 936 bp, Brachypodium distachyon
atggacatcctgcagaagcacggcgccgacgcccgcaaccgcaacaacaacaaccagcaagcgcccatccgcctttccctctcccctccgggccgggttccggtgtccccgccgatgtccccgaactcggaggcggcgttgctggcgaacgcacgctggacgccgacccaggagcagcgggagctcctggaagggctgtaccgccaggggctgcacaccccgagcgcggagcagatccaggggatcgcggccaggctgcggcagcacggccccgtcgagggtaagaatgtcttctactggttccagaactacaaggcccgccagcgccagcgccagaggctgcagggcctcgcctacttcgacagggaattccgccgccccatgccgatccccgtgctccacaggttcccctcgccgccggcgacggctcctgttcctcttcttcctgctgcatgcaacagatcggaagcgaatatgtacaggcagccaagcttcttcccgcagacgccacaggccgcggcaaatgcaactgcagcccactacttgcagactcaaccgccgctgctgtaccctggattcggaaacgccccggcgctctcaaggtaccagcagccagctccgaacagcgccggcggcagcggcactcaacaactgcgcgggatgcgcttcccttcggccagtggggcggcgaacgccagctccggatccggcacccgtgaccgtgagaccctgcagctgttcccgctgcagcccacctgctggcagatgcgcgagaagaagaagaaaaactgcagcagcaccgggagcggctccccgagtccaatgacgccctccagctccggctcagcctcctcgtcgttctctttggagccggagagcccggaagtgccgttctatgacttcttcggccttcagtctggaggccgcgctgattaa

SEQ ID NO: 27 - protein, 311 aa, Brachypodium distachyon
MDILQKHGADARNRNNNNQQAPIRLSLSPPGRVPVSPPMSPNSEAALLANARWTPTQEQRELLEGLYRQGLHTPSAEQIQGIAARLRQHGPVEGKNVFYWFQNYKARQRQRQRLQGLAYFDREFRRPMPIPVLHRFPSPPATAPVPLLPAACNRSEANMYRQPSFFPQTPQAAANATAAHYLQTQPPLLYPGFGNAPALSRYQQPAPNSAGGSGTQQLRGMRFPSASGAANASSGSGTRDRETLQLFPLQPTCWQMREKKKKNCSSTGSGSPSPMTPSSSGSASSSFSLEPESPEVPFYDFFGLQSGGRAD

SEQ ID NO: 28 - DNA, 975 bp, Zea mays
atggagacgccacagcagcaatccgccgccgccgccgccgccgccgcccacgggcaggacgacggcgggtcgccgccgatgtcgccggcctccgccgcggcggcggcgctggcgaacgcgcggtggaacccgaccaaggagcaggtggccgtgctggaggggctgtacgagcacggcctgcgcacccccagcgcggagcagatacagcagatcacgggcaggctgcgggagcacggcgccatcgagggcaagaacgtcttctactggttccagaaccacaaggcccgccagcgccagaggcagaagcaggacagcttcgcctacttcagcaggctcctccgccggcccccgccgctgcccgtgctctccatgccccccgcgccaccgtaccatcacgcccgcgtcccggcgccgcccgcgataccgatgccgatggcgccgccgccgcccgctgcatgcaacgacaacggcggcgcgcgtgtgatctacaggaacccattctacgtggctgcgccgcaggcgccccctgcaaatgccgcctactactacccacagccacagcagcagcagcagcagcaggtgacagtcatgtaccagtacccgagaatggaggtagccggccaggacaagatgatgaccagggccgcggcgcaccagcagcagcagcacaacggcgccgggcaacaaccgggacgcgccggccaccccagccgcgagacgctccagctgttcccgctccagcccaccttcgtgctgcggcacgacaaggggcgcgccgccaacggcagtaataacgactccctgacgtcgacgtcgacggcgactgcgacagcgacagcgacagcgacagcgtccgcttccatctccgaggactcggatggcctggagagcggcagctccggcaagggcgtcgaggaggcgcccgcgctgccgttctatgacttcttcgggctccagtcctccggaggccgctga

SEQ ID NO: 29 - protein, 324 aa, Zea mays
METPQQQSAAAAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMAPPPPAACNDNGGARVIYRNPFYVAAPQAPPANAAYYYPQPQQQQQQQVTVMYQYPRMEVAGQDKMMTRAAAHQQQQHNGAGQQPGRAGHPSRETLQLFPLQPTFVLRHDKGRAANGSNNDSLTSTSTATATATATATASASISEDSDGLESGSSGKGVEEAPALPFYDFFGLQSSGGR

SEQ ID NO: 30 - DNA, 477 bp, Zea mays
atggacatggagatcccacagcagcaagccgcccacggccaggacgagagcgggccggcggcgccgccgctgtccccggcctccgccgcggcggccgcgcggtggaacccgaccaaggagcaggtggccgtgctggaggggctgtacgagcacggcctgcgcagccccagcgcggagcagatacagcagatcgcggacaggctgcgggagcacgggcacgggcacggcgccatcgagggcaagagcgtcttctactggttccagaaccacagggcccgcctgcgccagcagaggcagaagcaggaaagcttcgcctacttcaccaggctcctccgccggcccccgccgctgcccgtgctctccatgccccccgcgccaccgtaccgtcacggccgtgtcccgtccccggccacgatcacgatgccggcgccgccgccgccgacgcccgctgcatgctacaacagcggcggatcgcgt

SEQ ID NO: 31 - protein, 159 aa, Zea mays
MDMEIPQQQAAHGQDESGPAAPPLSPASAAAAARWNPTKEQVAVLEGLYEHGLRSPSAEQIQQIADRLREHGHGHGAIEGKSVFYWFQNHRARLRQQRQKQESFAYFTRLLRRPPPLPVLSMPPAPPYRHGRVPSPATITMPAPPPPTPAACYNSGGSR

SEQ ID NO: 32 - DNA, 942 bp, Oryza sativa
atggagacgacgacgacgacgttgggcggcggcggcggcgggcgggcgggaggcttctccgatccgccgtctccgctctcgccgccgctgtcgccggcctcggcggcggcggcggcgctggcgaacgcgcggtggacgccgaccaaggagcagatcgcggtgctggaggggctgtaccggcaggggctgcgcacgccgaccgccgagcagatacagcagatcacggcgaggctccgggagcacggccacattgagggcaagaacgtgttctactggttccagaaccacaaggcccggcagcggcagaagcagaagcagcagagcttcgactacttcagcaagctgttccgccgcccgccgccgctgcccgtgctccacaggccactcgcgcggcccttccctctcgccatggcgccgacggcgatgccaccgccgccgccgccgccggcgacgacgacgacggccgcatgcaacgccggtggtgtgatgttcaggacgccaagcttcatgccggtcgcgacaaataacgccagctactacccgcagcagcagacgccgttgctgtacccagggatggaagtgtgtccgcacgacaagtccacggcgcagccaccggccaccaccaccatgtacctgcaggcaccgccgagcagcgcacacctcgcggcggcggctggccgcggcgcggcggaagcggaaggccatggccgccgcggcggcggcgccggtgggcgcgagaccctccagctgttccccctgcagcccaccttcgtgctgccggatcacaagccgctccgcgccgggagcgcctgcgccgccgtgtccccgacgacgccgtccgcgtccgcgtcgttctcgtgggagtcggagagctcggacagccccagcagcgaggcgcctccgttctacgacttcttcggcgtccattctggaggccgctga

SEQ ID NO: 33 - protein, 313 aa, Oryza sativa
METTTTTLGGGGGGRAGGFSDPPSPLSPPLSPASAAAAALANARWTPTKEQIAVLEGLYRQGLRTPTAEQIQQITARLREHGHIEGKNVFYWFQNHKARQRQKQKQQSFDYFSKLFRRPPPLPVLHRPLARPFPLAMAPTAMPPPPPPPATTTTAACNAGGVMFRTPSFMPVATNNASYYPQQQTPLLYPGMEVCPHDKSTAQPPATTTMYLQAPPSSAHLAAAAGRGAAEAEGHGRRGGGAGGRETLQLFPLQPTFVLPDHKPLRAGSACAAVSPTTPSASASFSWESESSDSPSSEAPPFYDFFGVHSGGR

SEQ ID NO: 34 - DNA, 774 bp, Medicago truncatula
atggagggtgagaataataatgagatgaatgaagaggggtcgaatgggaataatagtatttcatcaagttcacgatggaacccaacaaaagaacaaataagtatgttggagaatttgtataagcaaggaataaagacaccaagtgctgaagaaatacaagaaatcactgctaggcttagagtgtatggtcacatcgaaggaaagaatgtcttttactggtttcagaatcacaaagcaagacagagacagaaacagaagcaagaaagtattgcttatttcaatcgacttcttcatagacctcaacccatttatccttcacctatttgtccaaatgctatgcgtgcaccatattgcgtaccacaaccacaaagtcatgaaataagctattaccctcaaaatccaaaagtgcttctacctgcagtaggttatagaaggaaccatgctgagaaagttatgtcaaactcaaatggcatgtcaaatatctgcaatagtccaatggtatataatgaaaacatgcaacagagaatcctaaaccctaattatgactttggttacagtaatcaagaaacattggatttgtttcctctgcatccaactggcatattggaagggaaatcaactgaccaggtgtcttctattgtttcagtttctgctgatagttccactgatacacattctggttcttctcatcatgagatcaatcaagatcatggttataatgggaacaaacccttctttgacttttttaataattctgcaggacaaggttcttag

SEQ ID NO: 35 - protein, 257 aa, Medicago truncatula
MEGENNNEMNEEGSNGNNSISSSSRWNPTKEQISMLENLYKQGIKTPSAEEIQEITARLRVYGHIEGKNVFYWFQNHKARQRQKQKQESIAYFNRLLHRPQPIYPSPICPNAMRAPYCVPQPQSHEISYYPQNPKVLLPAVGYRRNHAEKVMSNSNGMSNICNSPMVYNENMQQRILNPNYDFGYSNQETLDLFPLHPTGILEGKSTDQVSSIVSVSADSSTDTHSGSSHHEINQDHGYNGNKPFFDFFNNSAGQGS

SEQ ID NO: 36 - DNA, 672 bp, Phseolus vulgaris
atggagagtcgcacttcagcaggaagagaaccaagtgggagtctttcagtttcgaggtggagccctaagaaagagcaaatagcaatcttggagagcttttacaagcagggaataaggactccaaatactgagcagataaaagagattacctctagactcagtgcttatggtcacattcaaggaaaaaatgtcttctactggtttcagaatcataaagcccgtcaaagacagaagcagaagcaacaatgcattgcatattccaatagctttcttcatgcctcacaacccattggccaaactgttgtctgtgccccattttatcctcaacagtgtggaatgaacttctatcctcaaccaggaaagattgttccagcaggaggtgttattgagacagttgtgccctttgggatgctgaaaatgtgtgatgtccagcagaaacaattgcaacagagggtacaaacgggttataactatagcatcagtgacagcaaaaccttaactctcttccctcttcatccaacaggcatcctggaagaaaaaactaccaatcaagtgtcttcacatgcttcattagtttcagttgatgtcactgatacatgtcatggttctcttagtgttgttgaagatagccgtcctgaaaagcaacagttcttggactttttaactttttga

SEQ ID NO: 37 - protein, 223 aa, Phseolus vulgaris
MESRTSAGREPSGSLSVSRWSPKKEQIAILESFYKQGIRTPNTEQIKEITSRLSAYGHIQGKNVFYWFQNHKARQRQKQKQQCIAYSNSFLHASQPIGQTVVCAPFYPQQCGMNFYPQPGKIVPAGGVIETVVPFGMLKMCDVQQKQLQQRVQTGYNYSISDSKTLTLFPLHPTGILEEKTTNQVSSHASLVSVDVTDTCHGSLSVVEDSRPEKQQFLDFLTF

SEQ ID NO: 38 - DNA, 792 bp, Phseolus vulgaris
atggagagtgaaaatgaggagttggagatgggagggtctaacggggttgcagccagttcacggtggaaccctacaaaagagcaaataagcatgctagagaacctgtataagcagggaataaagactcccagtgctgaggagatacagcagataactgctaggcttagggcgtatggtcacatcgaaggaaagaatgtcttctactggtttcaaaatcacaaagctaggcagagacagaagcagaagcaagaaaccatcgcttacttcaatcgctttcttcatactccccagccctttttttctccgccaatttgcccaaatggcctgtgccctccatattgtgtacctcaggcgcagagtgaaaatggtttttaccccccacatccaaaggtgcttgtaccagtaggttttaggagaagcccgtctgagatatttgtgcccacagacatgtcaaacatgatcagcaatggccctctggcatacgaagccatgcaacagagaatctcggattgtaacttcagctacagtaaccaagaaaccttggatctctttcctctgcacccaactggtattttggaagggaaaacaacataccaggtgtcttctctggcttcagtttctgctgatagttccagtgatacacctcctgctccttctcctgatatcaatgaagatgatccttccccacttaaccaacccttctttgattttttactacttctggtcaaggttcttaagagacttcttttatgtcattggcttgaacttttaattcccatggctcatcactga

SEQ ID NO: 39 - protein, 263 aa, Phseolus vulgaris
MESENEELEMGGSNGVAASSRWNPTKEQISMLENLYKQGIKTPSAEEIQQITARLRAYGHIEGKNVFYWFQNHKARQRQKQKQETIAYFNRFLHTPQPFFSPPICPNGLCPPYCVPQAQSENGFYPPHPKVLVPVGFRRSPSEIFVPTDMSNMISNGPLAYEAMQQRISDCNFSYSNQETLDLFPLHPTGILEGKTTYQVSSLASVSADSSSDTPPAPSPDINEDDPSPLNQPFFDFLLLLVKVLKRLLLCHWLELLIPMAHH

SEQ ID NO: 40 - DNA, 1023 bp, Sorghum bicolor
atggagatgccaccgcagcaagccgccgccgccgccgccaacgccaacgccaacgccaacggcgccgcccacgggcaggacgagggcgggtcgtcgtcgccgccgctgtccccggcgtccgccgcgctggcgaacgcgcggtggaacccgaccaaggagcaggtggcggtgctggaggggctgtacgagcacggcctgcgcaacccgagcgcggagcagatacagcagatcacgggcaggctgcgggagcacggcgccatcgagggcaagaacgtcttctactggttccagaaccacaaggcacggcagcgccatcgccagaagcaggacagcttcgcctacttcaccacgctcctccgccggcccccgccgctgcccgtgctccccatgcagcccgcgccgccgtaccttcacggccgcgtcccgtccccgtccccgtctccggcgccggcgccgaccgcgatcgcgatgccgccgccaccgccaccgccgtcgcccgctgcatgcaacaacaacaacaacaacaacaacaacggcggcgcgcgtgtgatgtacaggaatgcattctacatgcctgcgccgcaggcgccccctgcaaatgctgcctactactacccgccaccgcagcagcaggtgacagtcatgtaccagtacccgagaatggagctcggccaggacaagatgatgatgaccacagccgcggcgcagcagtacccggccgtcatgttccagcaggccggcgccgcagctaccaatggcgccggaccagcggaacgcgtcggccccaggcgcgagacgctcgagctgttcccgctcaagcccaccttcgtgctgcggcgcgacaaggggcgcgctggcggtggcagcgcctccgccctgacgtcggcgtcgacggcgacggcatccgcttccgcctccgaggagtcggagggcctggagagcggcaactgcaacggcgaggagcaggaggcgcccgcgctgccgttctatgacttcttcggtctccagtccggcggccgctga

SEQ ID NO: 41 - protein, 340 aa, Sorghum bicolor
MEMPPQQAAAAAANANANANGAAHGQDEGGSSSPPLSPASAALANARWNPTKEQVAVLEGLYEHGLRNPSAEQIQQITGRLREHGAIEGKNVFYWFQNHKARQRHRQKQDSFAYFTTLLRRPPPLPVLPMQPAPPYLHGRVPSPSPSPAPAPTAIAMPPPPPPPSPAACNNNNNNNNNGGARVMYRNAFYMPAPQAPPANAAYYYPPPQQQVTVMYQYPRMELGQDKMMMTTAAAQQYPAVMFQQAGAAATNGAGPAERVGPRRETLELFPLKPTFVLRRDKGRAGGGSASALTSASTATASASASEESEGLESGNCNGEEQEAPALPFYDFFGLQSGGR

SEQ ID NO: 42 - DNA, 900 bp, Triticum aestivum
atgaagagcgtcgagcaccacgacgaagccgccaccggcctctccctttcccctccccctacgtcggcgccgctctccccaccgatatcctccaactccgcggcggtggcggcgctggcaaatgcgcgctgggtccctaccagggagcagatcgcggtgctggaggggctgtaccgccaggggatgcgcacccccaccgccgaggaggtacatcaggtgacggcgaggctgcaggagcacggccccatcgagggcaagaacgtcttctactggttccagaaccggctgcgccagaagcagaagcagcaacagagatccgactacttcgccaggcagctccgccgtccccagccgctgcccacgctccgcaggacccccggccactccttctccccggtccagctgcaggcgccgccggcgccgaacactcctgcatgcaacagagaagggatgtacatgcagcaaccatgttacatgacagggccagcagcacaagcctcggcgaatgcatcctactactcgcagatgcagccgtcgctaacgaacccaaatgtagggactatggcgcatggcaacatccaggcacaggcacaggccaccatgtacttccagacagcagcttcgaacaactccaacactcaacaaccgcatgcggtccagttcccttcaaccgacggcagctacggggctcctgacgcctactcccgtcgtcccgtgcttctgaatctgttcccgcaataccccaccttcgcgaatcgcgagaagacccgccacactgagagcgccggctccccaaggccatccacctcaaggtcgttctcttgggagtctgagagctctgagatccccagcggagatggctcgagatcgttctatgacttctttggtgaaggtcactga

SEQ ID NO: 43 - protein, 299 aa, Triticum aestivum
MKSVEHHDEAATGLSLSPPPTSAPLSPPISSNSAAVAALANARWVPTREQIAVLEGLYRQGMRTPTAEEVHQVTARLQEHGPIEGKNVFYWFQNRLRQKQKQQQRSDYFARQLRRPQPLPTLRRTPGHSFSPVQLQAPPAPNTPACNREGMYMQQPCYMTGPAAQASANASYYSQMQPSLTNPNVGTMAHGNIQAQAQATMYFQTAASNNSNTQQPHAVQFPSTDGSYGAPDAYSRRPVLLNLFPQYPTFANREKTRHTESAGSPRPSTSRSFSWESESSEIPSGDGSRSFYDFFGEGH

SEQ ID NO: 44 - DNA, 969 bp, Triticum aestivum
atggagagcgtcgagcaccagcagcaggccgccacccgctcccgctccccgtcccttccggcggcgccgccctccccgccgctgtcgcccaactcggcggcggcggcggcgctggcgaacgcgcggtggacgccgaccaaggagcaggtgggcgtgctggaggggctgtaccggcaggggctgcgcacccccaccgcggagcagatccagcaggtgacggcgcggctgcagaagcacggccccatcgagggcaagaacgtcttctactggttccagaaccacaaggcccgccagcgccagcgccagaagcagcaggccttcgactacttctccaagcagttccgccgcccccagccgctgcccgtgctccacaggcccgccgcccacccctccttgcccccgatcccgctgcacgcgccgccgccgccaccgtcgcacgtgtcgtcggcgacgacgccggatcctcccgctcctcctgcatgcaacagacaagcgatgtacgggcagcagcctagctacgtgacagcggcggcggcggtggcggcgacacaggcagcggcgaatgcatcctactacatgcagacgcaggcacaggcgccgatgctgcaccccagagtggaggcggtggcgcatgacaaggcccagacgcaggcacaggccaccatgtaccaccaggcagcagctccgaacagcgccggcactcaacagccgcgcgcgctacagctccctccggccgccggcacccatgggccgcccgccgcccgctcccgccgccccgagaccctgaacctgttcccgctgcaccccaccttcgccattccggagaagccgcgccccgccgggatcgccggctccgcgacgccgacggggccgtccgcctcggggtcggggtccttctcttgggaaccggagagcccccgcggcgacgcccctctgccgttgtatgacttcttcggcgcaggccgctga

SEQ ID NO: 45 - protein, 322 aa, Triticum aestivum
MESVEHQQQAATRSRSPSLPAAPPSPPLSPNSAAAAALANARWTPTKEQVGVLEGLYRQGLRTPTAEQIQQVTARLQKHGPIEGKNVFYWFQNHKARQRQRQKQQAFDYFSKQFRRPQPLPVLHRPAAHPSLPPIPLHAPPPPPSHVSSATTPDPPAPPACNRQAMYGQQPSYVTAAAAVAATQAAANASYYMQTQAQAPMLHPRVEAVAHDKAQTQAQATMYHQAAAPNSAGTQQPRALQLPPAAGTHGPPAARSRRPETLNLFPLHPTFAIPEKPRPAGIAGSATPTGPSASGSGSFSWEPESPRGDAPLPLYDFFGAGR

SEQ ID NO: 46 - DNA, 735 bp, Arabidopsis thaliana
atgagtcctgtggcttcaacgaggtggtgtccgacgccggagcaactgatgatcttggaagagatgtaccggagtggtatacggactccgaatgcggtgcagatacaacagatcacagctcacttggcgttctatggaagaatcgagggcaaaaacgtcttttactggttccagaaccataaggctagagatagacagaagctgaggaagaaactagccaagcaacttcaccagcaacaacatcaacttcaactccaacttcagcagatcaaaccaaaaccaatatcatcgatgatttctcaaccagttaataagaatatcatcgatcatcacaatccttatcatcatcaccatcataatcatcatcataatcatcatcgtccatatgatcatatgtcctttgattgctgctctcatccttctcccatgtgtcttcctcatcagggaactggagtaggagaagctccaagcaaagtgatgaatgaatattactgcaccaaaagtggagctgaagagatattgatgcaaaaatcaatcacgggtccaaactcatcgtacggtcgagattggatgatgatgatggatatgggcccacgaccatcatatccctcatcatcatcatcacccatttcatgttgtaacatgatgatgagcagtccaaagataccattgaaaaccctggaacttttcccaatctcatctatcaactccaaacaagacagtaccaaactttaa

SEQ ID NO: 47 - protein, 244 aa, Arabidopsis thaliana
MSPVASTRWCPTPEQLMILEEMYRSGIRTPNAVQIQQITAHLAFYGRIEGKNVFYWFQNHKARDRQKLRKKLAKQLHQQQHQLQLQLQQIKPKPISSMISQPVNKNIIDHHNPYHHHHHNHHHNHHRPYDHMSFDCCSHPSPMCLPHQGTGVGEAPSKVMNEYYCTKSGAEEILMQKSITGPNSSYGRDWMMMMDMGPRPSYPSSSSSPISCCNMMMSSPKIPLKTLELFPISSINSKQDSTKL

SEQ ID NO: 48 - DNA, 681 bp, Brassica rapa
atgagtccggtggcttcaacaaggtggtgtccgaccacggagcaactgatgatcttggaagaaatgtaccggaccggtatacggactcctaatgcggtgcagatacaacagatcacagctcacttagctttctatggacgtattgagggaaaaaacgtgttttactggtttcagaaccataaggcaagagatagacagaagctaaggaagaaactcgctaagcaacttcaccaacaacaactccaacaaattaagctcaaaccaatatcgatggtgtctgaaccacttaataacatcatcgatcatcacaatctttactaccataatcaccatcgtccgtatgatcattcgtctttcgcttgctgctctcagccttcttccgtttgtctttctcatcagggaattggagtagaagctcaaagcaaagtggtgaatgaatattattacaacaaaagtggacctgaagatatgttgatgcaaaaaccaatcacgggccaaaactcatcgtacggtcgagattggatggtgatgatggatatgggtccacgaccatcatattcctcattatcaccaattccatgtatcaacatgatgatgaacagtccaaagataccaccaaaaacccttgaactcttcccaatctcatccatcaactccaaacaagacagttccaaactctaa

SEQ ID NO: 49 - protein, 226 aa, Brassica rapa
MSPVASTRWCPTTEQLMILEEMYRTGIRTPNAVQIQQITAHLAFYGRIEGKNVFYWFQNHKARDRQKLRKKLAKQLHQQQLQQIKLKPISMVSEPLNNIIDHHNLYYHNHHRPYDHSSFACCSQPSSVCLSHQGIGVEAQSKVVNEYYYNKSGPEDMLMQKPITGQNSSYGRDWMVMMDMGPRPSYSSLSPIPCINMMMNSPKIPPKTLELFPISSINSKQDSSKL

SEQ ID NO: 50 - DNA, 705 bp, Brassica rapa
atgagtccagtggctgcaacgaggtggtgcccgacgcctgagcaactgatgatcttggaagagatgtaccggagcggcatacggactcctaatgcggtgcagatacaacaaatcacagctcatttagctttttatggacgtatcgaaggcaaaaacgtcttttactggtttcagaaccataaggcgagagatagacagaagctgaggaagaaactcgctaagcaacttcaccaacaacaacatcatctccaactccagctccaacacatccagcccaacccaatatctcaaccacttagtaacatcattgatcctcataatcattaccatcaccattataatcatcatcgtccttatgatcatatgtctttcgcttgctgctctcagccttctcccatttgtctttctcatcaggccattggagtagaagctcaaagcaaagtgctgagtgaacattactgcaacaaaagtggacgtgaggatatgttgatgcaaaaaccaatcacgggtcaaaacacttcgtacggtcgagattggatgatgatgatgatggatatgggcccacgaccatcatatccctcatcatcatcagtacccgttccatattgcaacatgatgatgaacagtccaaagataccactaaaaaccctcgaactcttcccaatctcatccatcaactccaaacaagacagttccaaactttaa

SEQ ID NO: 51 - protein, 234 aa, Brassica rapa
MSPVAATRWCPTPEQLMILEEMYRSGIRTPNAVQIQQITAHLAFYGRIEGKNVFYWFQNHKARDRQKLRKKLAKQLHQQQHHLQLQLQHIQPNPISQPLSNIIDPHNHYHHHYNHHRPYDHMSFACCSQPSPICLSHQAIGVEAQSKVLSEHYCNKSGREDMLMQKPITGQNTSYGRDWMMMMMDMGPRPSYPSSSSVPVPYCNMMMNSPKIPLKTLELFPISSINSKQDSSKL

SEQ ID NO: 52 - DNA, 828 bp, Brachypodium distachyon
atgccagcggcggcggcagtggcggcggtgtcgtcggcggcggcgacggggacgacgcggtggtgcccgacggcggagcagctgatggtgctggaggagatgtaccggggcgggctgcgcaccccgaacgcgtcgcagatccagcagatcacggcccacctggcccactacgggcggatcgaggggaagaacgtcttctactggttccagaaccacaaggcccgcgaccgccagaagctcaggcggaggctctgcatgacccaccaccttctctcctgcgccgccgccgccgccgcggtccagcaggcccagcaccaccactactacgctgccgctgccgctgcagggcagtttcttgggggcccagctggaggaggagccgggcatcccatggcccagcagcagcagcagcagtactactcggcggcgtcggcaatggcgtgccctgccggcggcttcgaccagcaccactaccagctgctcccggcgtcgcccatggccgccgcctgctacccggcccagctgcagcccagcagccgatgcgccggcggcgtctctccggtgccggcgccggcccagcccaaccagttcttgcagtaccagcagcagggaggagggtcgccggagttctcgctggggaggctggggagcttcggagtggtggtggaccaggaagaggccgacacgtgtcgctcgggatacgactaccagcagcagctggccgcggcgaggatggaagcagcagagacgacgtccttctgcaggccgccgctgaagacgctggagctcttccccggagcctccgtcaaggacgagcagctcgcctga

SEQ ID NO: 53 - protein, 275 aa, Brachypodium distachyon
MPAAAAVAAVSSAAATGTTRWCPTAEQLMVLEEMYRGGLRTPNASQIQQITAHLAHYGRIEGKNVFYWFQNHKARDRQKLRRRLCMTHHLLSCAAAAAAVQQAQHHHYYAAAAAAGQFLGGPAGGGAGHPMAQQQQQQYYSAASAMACPAGGFDQHHYQLLPASPMAAACYPAQLQPSSRCAGGVSPVPAPAQPNQFLQYQQQGGGSPEFSLGRLGSFGVVVDQEEADTCRSGYDYQQQLAAARMEAAETTSFCRPPLKTLELFPGASVKDEQLA

SEQ ID NO: 54 - DNA, 717 bp, Brachypodium distachyon
atgccgcagacgccgtcgactcggtggtgcccgacgccggagcagctgatgatcctggaggagatgtaccggagcggcgtgaggacgcccaacgcggcggagatccagcagatcacggcgcacctcgcctactacggccgcatcgagggcaagaacgtcttctactggttccagaaccacaaggcccgcgagcgacagcgcctccgccgccgcctctgcgcacgccaccagcagcagccgcctgcttcttctccgtctccggtcacacctcctcctccaaatgcttctgcaggagctgtcaatgtgatgcaccctgccgtgatgcagcttcaccatcaccaccaccaccacccctacgctaccacaaccacctgcagcttcatgccgcctcagggctactcgcaccagcagcagcaggatgcaggaggagctgttcccgtttcagggttggagtttccagcagctggtggaggcaaggctcatcagcagcagcaagaatggactacacagcagcagcaacagatgatgatgatggagaacagcaacaacattaacgctggaggcagctccggcgccggtggtatgaccccgccgccatggccatgctgccggccgcttagaacgcttgagctcttccctacaaagagcaccggcggtggcctcagggatgagtgcagcagctcaaagtcctcctcttgctcaacctccacaaactaa

SEQ ID NO: 55 - protein, 238 aa, Brachypodium distachyon
MPQTPSTRWCPTPEQLMILEEMYRSGVRTPNAAEIQQITAHLAYYGRIEGKNVFYWFQNHKARERQRLRRRLCARHQQQPPASSPSPVTPPPPNASAGAVNVMHPAVMQLHHHHHHHPYATTTTCSFMPPQGYSHQQQQDAGGAVPVSGLEFPAAGGGKAHQQQQEWTTQQQQQMMMMENSNNINAGGSSGAGGMTPPPWPCCRPLRTLELFPTKSTGGGLRDECSSSKSSSCSTSTN

SEQ ID NO: 56 - DNA, 789 bp, Zea mays
atgccgcagacaccttcgactcgctggtgcccgacgccggagcagctgatgatcctggaggagatgtaccggagcggcgtgaggacgcccaacgcggcggagatccagcagatcacggcgcacctggcctactacggccgcatcgagggcaagaacgtcttctactggttccagaaccacaaggcccgcgagcgccagcggctccgccgtcgcctctgcgcccggcaccagcagcagtacgcgcagcagcaggccaccgcggcggccccggcttcgagccctaacagcagcgccaccgttccgtccctcgcagcaggtggcagcagcgccggtgtgcatccggcggtgatgcagctgcaccatcaccagcacccgtacgcaaccaacttcatgccacaccagctgggctacatgggacagcaggtggcgactgttccgccagtgctgaacccagctgctgccggcatggtggaccttgcagctgcaagagcaggaggaggaaataaggctactgctgcaggtagtggtgcctatggaggtggagctgggttatacaacagctgcagcagcaatcagctggaggagtgggaggccacagatgcaatggagcactgcgacgccagctgcggtgcggcatcgggcagctctgacgagggtggcgcgctccagctgccgccatgctgccgccgtcctctaaagaccttggacctcttccccactaagagcactggactcaaggacgagtgcagcagctccaagtcctcctcttgctccacatccaccaactaa

SEQ ID NO: 57 - protein, 262 aa, Zea mays
MPQTPSTRWCPTPEQLMILEEMYRSGVRTPNAAEIQQITAHLAYYGRIEGKNVFYWFQNHKARERQRLRRRLCARHQQQYAQQQATAAAPASSPNSSATVPSLAAGGSSAGVHPAVMQLHHHQHPYATNFMPHQLGYMGQQVATVPPVLNPAAAGMVDLAAARAGGGNKATAAGSGAYGGGAGLYNSCSSNQLEEWEATDAMEHCDASCGAASGSSDEGGALQLPPCCRRPLKTLDLFPTKSTGLKDECSSSKSSSCSTSTN

SEQ ID NO: 58 - DNA, 777 bp, Zea mays
atgccgtcgcagcagcagcagcagcagggccagcaggtggcggcggggtcgacgcggtggtgcccgacgccggagcagctgatgatcctggaggacatgtaccgcggcggcctgcgcacgcccaacgcgtcgcagatccagcagatcacggcgcacctcgcctgctacggccgcatcgagggcaagaacgtcttctactggttccagaaccacaaggcccgggaccgccagaagatgcgccgcaggctctgcatgagccaccacctcctctcctgcgcgcagtactacgccgcggcggcgcaccacggccacgccgccttcctcgccgcgccgccgccgtacgggcaccagctgctctccccgtccacgacgtcgcccaccccggctgctgctgctgctgccgccgctgcagcgtacggctactactaccccgccacggccgccttcgctgcaccggcgagccggtgcgccggcaacgccactcccccgtcgccgaccacccagctgttccactatcagggtggcggagggcttgtgccgacggaggcgctcggccggccggagtactcgctggggaagctggacaacttcggcgtggcgcttgacgacgtcgtggtgagctcgacctccggtgctgtcgtcgacacgatgggggcgcctccggttgcagggttcgaggtggcgccgccgctgccggctgccttcagctgccggccgctcaagacgctagacctcttccccggcggtctcgaggaagagcagcacgacgtggcctga

SEQ ID NO: 59 - protein, 258 aa, Zea mays
MPSQQQQQQGQQVAAGSTRWCPTPEQLMILEDMYRGGLRTPNASQIQQITAHLACYGRIEGKNVFYWFQNHKARDRQKMRRRLCMSHHLLSCAQYYAAAAHHGHAAFLAAPPPYGHQLLSPSTTSPTPAAAAAAAAAAYGYYYPATAAFAAPASRCAGNATPPSPTTQLFHYQGGGGLVPTEALGRPEYSLGKLDNFGVALDDVVVSSTSGAVVDTMGAPPVAGFEVAPPLPAAFSCRPLKTLDLFPGGLEEEQHDVA

SEQ ID NO: 60 - DNA, 663 bp, Zea mays
atgccgtcgcagcagcagcagcagcaggcggcggcggggtcgacgcggtggtgtccgacgccggagcagctgatggtcttggaggagacgtaccggggcggcctgcgcacgcccaacgcgtcgcagatccagcagatcacggcgcacctcgcctgctacggccgcatcgagggcaagaacgtcttctactggttccagaaccacaaggcccgggaccgccagaagctgcgccgcatgctctttatgagccagagccaccacctcctctcctgcgcgcagtactacgctgcggtactggcgccgcgccacggacaccagttgttgctctccccgtcatcgacgtcgcccaccccaccagctgctgctgccgctgcggcgtacggctactactactccgccacggccttcgctgaaccggcgagcagcggaggagggcttgtgccggcggaggcgctcggccggccagagtactcgtcgctggacaacttcggcgtggcgctcgacgatgtcgtggtgagctccgcctccgccgccgtcgaaatgacgcctccggggttcgaggtggtggtgccgccgccgccggctgctgccttctgccggccgctcaagacgttggacctcttcccctgcgggctcaaggaagagcagcacgacgtggcctga

SEQ ID NO: 61 - protein, 220 aa, Zea mays
MPSQQQQQQAAAGSTRWCPTPEQLMVLEETYRGGLRTPNASQIQQITAHLACYGRIEGKNVFYWFQNHKARDRQKLRRMLFMSQSHHLLSCAQYYAAVLAPRHGHQLLLSPSSTSPTPPAAAAAAAYGYYYSATAFAEPASSGGGLVPAEALGRPEYSSLDNFGVALDDVVVSSASAAVEMTPPGFEVVVPPPPAAAFCRPLKTLDLFPCGLKEEQHDVA

SEQ ID NO: 62 - DNA, 861 bp, Oryza sativa
atggcgccggcggtgcagcagcagcagagcggcggcggcggcggatcgacgggggcggcggcggtggggtcgacgacgcggtggtgcccgacgccggagcagctgatgatgctggaggagatgtacaggggagggctccggacgccgaacgcggcgcagatacagcagatcacggcgcacctctcgacgtacggccgcatcgagggcaagaacgtcttctactggttccagaaccacaaggcccgcgaccgccagaagctccgccgccgcctctgcatctcccaccacctcctctcctgcgcccactactaccaccaccacctcgccgccgccgccgccgtcgttccgccgccgcagcttctgccgccgctgcacccctcctcctcctcctcctcctgcggcggtggcctcatcgaccacgctaattcccttctctcccccacgtcggcgaccacccccacctccgccgccgcagcagcagcagcagcagcttacaccaccagctactactaccccttcaccgccgccgccgcaccgccaccgcccaggacgtcgccggcggcgagccccctcttccactacaaccagggaggcggcggcgtggtgttgccggcggcggaggcgatcgggcgttcgtcgtcgtcgtcggactactcgctggggaagctagtggacaacttcggggtggcgctggaggagacgttcccggcgcagccgcagcagccggcgacgacgatggcgatgacggccgtcgtcgacactacggcggtggcggcggcggcaggtggcttctgccggccgctcaagacgctggacctcttccccggcggcctcaaggaagagcagcatgacgtcgtctag

SEQ ID NO: 63 - protein, 286 aa, Oryza sativa
MAPAVQQQQSGGGGGSTGAAAVGSTTRWCPTPEQLMMLEEMYRGGLRTPNAAQIQQITAHLSTYGRIEGKNVFYWFQNHKARDRQKLRRRLCISHHLLSCAHYYHHHLAAAAAVVPPPQLLPPLHPSSSSSSCGGGLIDHANSLLSPTSATTPTSAAAAAAAAAYTTSYYYPFTAAAAPPPPRTSPAASPLFHYNQGGGGVVLPAAEAIGRSSSSSDYSLGKLVDNFGVALEETFPAQPQQPATTMAMTAVVDTTAVAAAAGGFCRPLKTLDLFPGGLKEEQHDVV

SEQ ID NO: 64 - DNA, 705 bp, Oryza sativa
atgcctcagaccccttcgacgcggtggtgcccgacgccggagcagctgatgatcctggaggagatgtacaggagcggcgtgcgaacgcccaacgcggcagagatccagcaaatcacggcgcacctcgcctactacggccgcatcgagggcaagaacgtcttctactggttccagaaccacaaggcccgcgagcgccagcgcctccgccgccgcctctgcgcgcggcaccagcagcaaccctcaccgccctcctccacggtgcctccggctcccactgctgctgctgccggtgccgtcgtgcaggtgcaccccgcggtgatgcagctacaccaccaccaccaccaccatcacccatacgctgcggccgccgctgcccaaagtcatcacctgcagcagcagcagcagcagcaagctgagtggccggcggcggtggactactgcagcactgcatcggcgtcagcgtcggcaactgctgctgacatggcgatcccgccgtgctgccggccgctgaaaacgttggagctgttcccgaccaagagcaccagcggcggcctcaaggaagattgctgcagcagctccaaaccatgggcgcgctcagtggtcgagtcgctaggagggagggagaggagcagtttggcggtggttgacagtggtggcgacatggatggaatagagcgaaggaggagagaattgggagagaggtag

SEQ ID NO: 65 - protein, 234 aa, Oryza sativa
MPQTPSTRWCPTPEQLMILEEMYRSGVRTPNAAEIQQITAHLAYYGRIEGKNVFYWFQNHKARERQRLRRRLCARHQQQPSPPSSTVPPAPTAAAAGAVVQVHPAVMQLHHHHHHHHPYAAAAAAQSHHLQQQQQQQAEWPAAVDYCSTASASASATAADMAIPPCCRPLKTLELFPTKSTSGGLKEDCCSSSKPWARSVVESLGGRERSSLAVVDSGGDMDGIERRRRELGER

SEQ ID NO: 66 - DNA, 612 bp, Oryza sativa
atgcctcagaccccttcgacgcggtggtgcccgacgccggagcagctgatgatcctggaggagatgtacaggagcggcgtgcgaacgcccaacgcggcagagatccagcaaatcacggcgcacctcgcctactacggccgcatcgagggcaagaacgtcttctactggttccagaaccacaaggcccgcgagcgccagcgcctccgccgccgcctctgcgcgcggcaccagcagcaaccctcaccgccctcctccacggtgcctccggctcccactgctgctgctgccggtgccgtcgtgcaggtgcaccccgcggtgatgcagctacaccaccaccaccaccaccatcacccatacgctgcggccgccgctgcccaaagtcatcacctgcagcagcagcagcagcagcaagctgagtggccggcggcggtggactactgcagcactgcatcggcgtcagcgtcggcaactgctgctgacatggcgatcccgccgtgctgccggccgctgaaaacgttggagctgttcccgaccaagagcaccagcggcggcctcaaggaagattgctgcagcagctccaagtcctcctcttgctccacctccaccaattaa

SEQ ID NO: 67 - protein, 203 aa, Oryza sativa
MPQTPSTRWCPTPEQLMILEEMYRSGVRTPNAAEIQQITAHLAYYGRIEGKNVFYWFQNHKARERQRLRRRLCARHQQQPSPPSSTVPPAPTAAAAGAVVQVHPAVMQLHHHHHHHHPYAAAAAAQSHHLQQQQQQQAEWPAAVDYCSTASASASATAADMAIPPCCRPLKTLELFPTKSTSGGLKEDCCSSSKSSSCSTSTN

SEQ ID NO: 68 - DNA, 621 bp, Medicago truncatula
atgtctccacctggttcatcaagatggagccctacaactgaacaacttatgatcttagaggaactgtatagaagtggaattagaactcctagtgctgttcaaattcagcaaataactactcacctttctttctatggtaggatagaaggaaaaaatgttttctattggtttcagaatcataaggctagggatagacagaagttaagaaggaaacttaacaaacaactgcagctacagcagcagcagcagcagcaacaacaacaacaactacatgttcatcattgtcaactcaatcataataatatgattagtaaccattttgttggtagcacttttggttacccttttggatctaatgcacaagacttttccttttttaactcacctagcttgctttttgagggatcagtatctgcaaatacatcagaaggggaaataaattgcaaatggaatggttataataaccaacaaagtttgatggagaagaaagatattgcattcagcaactatggttggacgacactcatggataatgttaatcatgaagcaccttcatgttgcactaaacctctcaagactcttgatctttttccactcacaacaacaagaatcaaagactag

SEQ ID NO: 69 - protein, 206 aa, Medicago truncatula
MSPPGSSRWSPTTEQLMILEELYRSGIRTPSAVQIQQITTHLSFYGRIEGKNVFYWFQNHKARDRQKLRRKLNKQLQLQQQQQQQQQQQLHVHHCQLNHNNMISNHFVGSTFGYPFGSNAQDFSFFNSPSLLFEGSVSANTSEGEINCKWNGYNNQQSLMEKKDIAFSNYGWTTLMDNVNHEAPSCCTKPLKTLDLFPLTTTRIKD

SEQ ID NO: 70 - DNA, 612 bp, Phseolus vulgaris
atgtccccagcaggttcttcaagatggagccctaccacagaacaactcatgatcttagaggaactctacaggagtggcattagaactcctagtgcctctcaaattcagcaaataacgacacacctttctttctatggcaggatagaaggaaagaatgtgttctactggtttcagaatcacaaggctagggatagacagaagctcagaaggaaactcagcaagcaactgcagctacagcaacaacaactccaagtccatcattgccaactcaatcctgatatccctaaccattatgttggcacctttggctatactccttgttcttcaaccccggattttcccttttataacccacctacctttctttatcagggaggatcttcaaacacatctgaacaggcattgaattgcgcatggaatgtgcacaacctgcaaagtaatctggcggagaaggaagaggtgaccttctacaacaatggttggagtacaccagtggatgttaacaaccaagaatcttcatcatgttgcacttctcgacctctcaagacccttgatctctttccactcacaaccaccagaaccaatgaagactgcaccactccacccaagtag

SEQ ID NO: 71 - protein, 203 aa, Phseolus vulgaris
MSPAGSSRWSPTTEQLMILEELYRSGIRTPSASQIQQITTHLSFYGRIEGKNVFYWFQNHKARDRQKLRRKLSKQLQLQQQQLQVHHCQLNPDIPNHYVGTFGYTPCSSTPDFPFYNPPTFLYQGGSSNTSEQALNCAWNVHNLQSNLAEKEEVTFYNNGWSTPVDVNNQESSSCCTSRPLKTLDLFPLTTTRTNEDCTTPPK

SEQ ID NO: 72 - DNA, 783 bp, Sorghum bicolor
atgccgcagacaccttcgacccgctggtgcccgacgccggagcagctgatgatcctggaggagatgtaccggagcggcgtgaggacgcccaacgcggcggagatccagcagatcacggcgcacctggcctactacggccgcatcgagggcaagaacgtcttctactggttccagaaccacaaggcccgcgagcgccagcgcctccgccgccgcctctgtgcccgacaccagcagcagtacgctcagcagcagcagcagcaggccaccgcagcagcgccgtcttcgagccctcttccgtctccagcagcaggcggcagcagcgctggtgtgcatccggcggtgatgcagctgcaccagcaccaccacccatacgcaaccaacttcgtgccacacctgggctacttgggacagcaggcggcgactgttccgccagtgctgaacccagctgctgccggcatggtggaccttgcagctgcaggagcaggagcaggaaataaggctactggtgcaggtggtggctatggaggtggagctgccttatacaacagctgcagcagcaatcagctggaggagtgggaccccgctgaggccatggagcactgcaacgctagctgcggtgccgcatcgggcagctccgacgagggtggcgcagctcatctccagctgccgccatgctgccgccgccctctcaagaccttggacctgttccccactaagagcactgggctcaaggacgagtgcagcagctccaagtcctcctcttgctccacatccaccaactaa

SEQ ID NO: 73 - protein, 260 aa, Sorghum bicolor
MPQTPSTRWCPTPEQLMILEEMYRSGVRTPNAAEIQQITAHLAYYGRIEGKNVFYWFQNHKARERQRLRRRLCARHQQQYAQQQQQQATAAAPSSSPLPSPAAGGSSAGVHPAVMQLHQHHHPYATNFVPHLGYLGQQAATVPPVLNPAAAGMVDLAAAGAGAGNKATGAGGGYGGGAALYNSCSSNQLEEWDPAEAMEHCNASCGAASGSSDEGGAAHLQLPPCCRRPLKTLDLFPTKSTGLKDECSSSKSSSCSTSTN

SEQ ID NO: 74 - DNA, 819 bp, Sorghum bicolor
atgccgtcgcagcagcagcagcagcagggccagcgaggtgagccgcaggtggtggcggggtcgacgcggtggtgcccgacgccggagcagctgatgatcctggaggagatgtaccgtggcggcctgcgcacgcccaacgcgtcgcagatccagcagatcacggcgcacctcgcctgctacggccgcatcgagggcaagaacgtcttctactggttccagaaccacaaggcccgcgaccgccagaagctccgccgcaggctctgcatgagccaccacctcctctcctgcgcccagtactacgccgccgcgcaggcgcaccacggccacggccacggcggcttcctcgccgccgcgccgccgccggtcgtcgcgccgtatatgctctccccgtcgacgtcgcccaccccggctgctgctgctgctgccgctgcggcgtacggctactactaccccaccacggccgccgccgccttcgctgcaccaccggcgccgacggcgagcaggtgcgccggaaacgccacccctccgtcgccgacgacccagctcttccactatcagcatcagggtggcggtggaggagggcttgtgccggcggctgaggcgctcggccggccggagtactcgtcgctggggaagctggacaacttcggcgtggcggcgcttgacgacgtcgtcgtgagctccacctccacggccgtcgacatgatggcgcctcctgggttcgaggtggcgccgccgccggctgccttctgccggccgctcaagacgctggacctcttccccggcgggctcaaagaagagcagcacgacgtggcctga

SEQ ID NO: 75 - protein, 272 aa, Sorghum bicolor
MPSQQQQQQGQRGEPQVVAGSTRWCPTPEQLMILEEMYRGGLRTPNASQIQQITAHLACYGRIEGKNVFYWFQNHKARDRQKLRRRLCMSHHLLSCAQYYAAAQAHHGHGHGGFLAAAPPPVVAPYMLSPSTSPTPAAAAAAAAAYGYYYPTTAAAAFAAPPAPTASRCAGNATPPSPTTQLFHYQHQGGGGGGLVPAAEALGRPEYSSLGKLDNFGVAALDDVVVSSTSTAVDMMAPPGFEVAPPPAAFCRPLKTLDLFPGGLKEEQHDVA

SEQ ID NO: 76 - DNA, 822 bp, Triticum aestivum
atggcggcgacggcgactgcgacggcggcggcgacgagcgtggtgacggggacgacgcggtggtgcccgacgccggagcagctgatgatcctggaggagatgtaccgcggcgggctgcgcacccccaacgcgtcgcagatccagcagatcacggcgcacctggcccactacggccgcatcgagggcaagaacgtcttctactggttccagaaccacaaggcccgggaccgccagaagctccgccgcaggctctgcatgagccaccacctcctctcctgcgcgcactactacgccgccgccaacgccggccagtaccaccagcagcagcagctcctcggcgccggcgcggtgcctcctccgctgctgcagcacccgcagcagcagcagtactactccgcctcttgcgccggtggcggctacgaccagcacctgctcccgacgaccgtcccagcttccgcttacgctgctgctgctgggtacgcctaccccttcgccggcgtgccggcaagccggtgcgccgagccctcgccgccaaacaccccgctctccttccatcatcagctgacctgggttcatactattgtgcgtgcagggaggaggcgtagtgggatcgccggagtactcgctggggaggctgggcaacttcggcgtggtggaggacacatgccggccgtcgcggtacgagcagcagccgcagcagctggccgcggcgacggaagatcaggcggcgccggtgacggcgacggggctgttctgccggccgctgaagacgctggacctcttccccggcgcgatcaaggaggagcagcgcgacgtcgccta

SEQ ID NO: 77 - protein, 274 aa, Triticum aestivum
MAATATATAAATSVVTGTTRWCPTPEQLMILEEMYRGGLRTPNASQIQQITAHLAHYGRIEGKNVFYWFQNHKARDRQKLRRRLCMSHHLLSCAHYYAAANAGQYHQQQQLLGAGAVPPPLLQHPQQQQYYSASCAGGGYDQHLLPTTVPASAYAAAAGYAYPFAGVPASRCAEPSPPNTPLSFHHQLTWVHTIVRAGRRRSGIAGVLAGEAGQLRRGGGHMPAVAVRAAAAAAGRGDGRSGGAGDGDGAVLPAAEDAGPLPRRDQGGAARRRL

SEQ ID NO: 78 - DNA, 795 bp, Triticum aestivum
atggcggcgacggcgactgcgacggcggcggcgacgagcgtggtgacggggacgacgcggtggtgcccgacgccggagcagctgatgatcctggaggagatgtaccgcggcgggctgcgcacccccaacgcgtcgcagatccagcagatcacggcgcacctggtccactacggccgcatcgagggcaagaacgtcttctactggttccagaaccacaaggcccgggaccgccagaagctccgccgcaggctctgcatgagccaccacctcctctcctgcgcccactactacgccgccgccaacgccggccagtaccaccaccagcagcagctcctcggcgccggcgcggttccccctccgctgctgcagcaccagcagcagcagcagtactactccgcctcctgcgccggcggcagctacgaccagcacctgctcccgacgaccgtcccagcttccgcttatgctgctgctgctgctgggtacgcctaccccttcgccgccgtgccggcgagccggtgcgccgacccctcgccgcccaacacgccgctgtccttccatcaccagggtggaggcgtagtaggatcgccggagtactcactggggaggctgggcaacttcggcgtggtggacgacacgtgccggccgtcgcggtgcgagcagcagccacagcagctggccgtggcgacggaagatcaggcggcgccggtgacggcgacggggctgttctgccggccgctgaagacgctggacctcttccccggcgcgatcaaggaggagcagcgcgatgtcgcctag

SEQ ID NO: 79 - protein, 264 aa, Triticum aestivum
MAATATATAAATSVVTGTTRWCPTPEQLMILEEMYRGGLRTPNASQIQQITAHLVHYGRIEGKNVFYWFQNHKARDRQKLRRRLCMSHHLLSCAHYYAAANAGQYHHQQQLLGAGAVPPPLLQHQQQQQYYSASCAGGSYDQHLLPTTVPASAYAAAAAGYAYPFAAVPASRCADPSPPNTPLSFHHQGGGVVGSPEYSLGRLGNFGVVDDTCRPSRCEQQPQQLAVATEDQAAPVTATGLFCRPLKTLDLFPGAIKEEQRDVA

SEQ ID NO: 80 - DNA, 804 bp, Triticum aestivum
atggcggcgacggcgactgcgacggcggcggcgacgagcgtggtgacggggacgacgcggtggtgcccgacgcccgagcagctgatgatcctggaggagatgtaccgcggcgggctgcgcacccccaacgcgtcgcagatccagcagatcacggcgcacctggcccactacggccgcatcgagggcaagaacgtcttctactggttccagaaccacaaggcccgggaccgccagaagctccgccgcaggctctgcatgagccaccacctcctctcctgcgcgcactactacgccgccgccaacgccggccagtaccaccaccagcagcagctcctcggcgccggcgcggtcccccctccgctgctgcagcaccagcagcagcagcagcagtactactccgcctcctgcgccggcggcggctacgaccagcacctcctcccgacgaccgtcccagcttccgcttacgctactgctgctgggtacgcctaccccttcgccgccgtgccggcgagccggtgcgccgacccctcgccgcccaaaacgccgttgtccttccatcaccagggtggaggcatcgtaggatcgccggagtactcgctggggaggctgggcaacttcggcgtggtggacgacacgtgccggccgtcgcggtacgagcagcagcagcagcagcagcagctgaccgcggcgacggaagatcaggcggcgccggtgacggcgacgacggggctgttctgccggccgctgaagacgctggacctcttccccggcgcgatcaaggaggagcatcgcgacgtcgcctag

SEQ ID NO: 81 - protein, 267 aa, Triticum aestivum
MAATATATAAATSVVTGTTRWCPTPEQLMILEEMYRGGLRTPNASQIQQITAHLAHYGRIEGKNVFYWFQNHKARDRQKLRRRLCMSHHLLSCAHYYAAANAGQYHHQQQLLGAGAVPPPLLQHQQQQQQYYSASCAGGGYDQHLLPTTVPASAYATAAGYAYPFAAVPASRCADPSPPKTPLSFHHQGGGIVGSPEYSLGRLGNFGVVDDTCRPSRYEQQQQQQQLTAATEDQAAPVTATTGLFCRPLKTLDLFPGAIKEEHRDVA

SEQ ID NO: 82 - DNA, 726 bp, Triticum aestivum
atgccgcagacgccatcgacccgttggtgcccgacgcctgagcagctgatgatcctggaggagatgtaccggagcggcgtgcgcacacctaacgcggcggagatccagcagatcacggcgcacctcgcctactacggccgcatcgagggaaagaacgtcttctactggttccagaaccacaaagcccgcgagcgccagcgtctccgtcgccgcctctgcgcccgccaccagcaaccctcctcgccggcggctcctcctcctcctcctcctcatactggtgctgccggtggcggaggcaatgctgctggtgctggtgctggcgtgaacgcgatgcaccccgcggtgatgcagctgcaccatcaccaccacacatacgctaccagctgcttcatggcgcctcagggctacttgcagcaggaaacagcagcagcaggagctcttccagtttcggggttggagtttgcaggcaagacaagccagcagcaggaatggatgatgcagcagcagatggtgatggacaacagcaacattaacaacagtgtagcagcagctggaggcagctccgcagcggccggcggtggtatgaataatatgaccccaccgccatggccatgctgccggccgctcagaaccctagagctcttccctacaaagagcaccggcggcggcctcagggacgagtgcagcagctccaagtcctcctcttgttccacctccacaaactag

SEQ ID NO: 83 - protein, 241 aa, Triticum aestivum
MPQTPSTRWCPTPEQLMILEEMYRSGVRTPNAAEIQQITAHLAYYGRIEGKNVFYWFQNHKARERQRLRRRLCARHQQPSSPAAPPPPPPHTGAAGGGGNAAGAGAGVNAMHPAVMQLHHHHHTYATSCFMAPQGYLQQETAAAGALPVSGLEFAGKTSQQQEWMMQQQMVMDNSNINNSVAAAGGSSAAAGGGMNNMTPPPWPCCRPLRTLELFPTKSTGGGLRDECSSSKSSSCSTSTN

SEQ ID NO: 84 - DNA, 729 bp, Triticum aestivum
atgccgcagacgccatcgacccgttggtgcccgacgcctgagcagctgatgatcctggaggagatgtaccggagcggcgtgcgcacacctaacgcggcggagatccagcagatcacggcgcacctcgcctactacggccgcatcgagggaaagaacgtcttctactggttccagaaccacaaggcccgcgagcgccagcgtctccgtcgccgcctctgcgcccgccaccagcaaccctcctccccggcggctcctcctcctcctcctcctcctcatactggtgctgccggtggcggaggcaatgctgctggtgctggtgcgggcgtgaacgtgatgcaccccgcggtgatgcagctgcaccatcaccaccacacatacgctaccagctgcttcatggcgcctcagggctacttggagcaggaaacagcagcagcaggagctcttccagtttcggggttggagtttgcaggcaagacaagccagcagcaggaatggatggcgcaggagcagatggtgatggagaacagcaacattaacaacagtgtagcagcagctggaggcagctccgcatcggccggcggtggtatgaataatatgaccccgccgccatggccatgctgccggccgctcagaaccctagagctcttccctacaaagagcaccggtggcggcctcagggacgagtgcagcagctccaagtcctcctcttgctccacctccacaaactag

SEQ ID NO: 85 - protein, 242 aa, Triticum aestivum
MPQTPSTRWCPTPEQLMILEEMYRSGVRTPNAAEIQQITAHLAYYGRIEGKNVFYWFQNHKARERQRLRRRLCARHQQPSSPAAPPPPPPPHTGAAGGGGNAAGAGAGVNVMHPAVMQLHHHHHTYATSCFMAPQGYLEQETAAAGALPVSGLEFAGKTSQQQEWMAQEQMVMENSNINNSVAAAGGSSASAGGGMNNMTPPPWPCCRPLRTLELFPTKSTGGGLRDECSSSKSSSCSTSTN

SEQ ID NO: 86 - DNA, 756 bp, Arabidopsis thaliana
atgaaggttcatgagttttcgaatgggttttcttcatcgtgggatcaacatgactcgacatcatcccttagcctaagctgcaaacgcctccgtcctctcgcccctaagctctccggcagccctccctcccctccttcttcttcctccggcgtcacttcagccacttttgaccttaaaaacttcattagacccgatcaaaccggtccgacaaaatttgaacacaaacgagaccctcctcatcaattggagacgcacccgggagggacaaggtggaacccgactcaagaacagatagggatacttgagatgttgtacaaaggtggaatgcgtactcctaatgctcaacagattgagcatatcacattgcaactcggtaagtacgggaaaatcgaagggaaaaatgtgttctattggttccagaaccacaaagcccgcgagagacagaagcagaagaggaacaacctcatcagcctaagttgccaaagcagcttcacgaccactggtgtctttaatccgagtgtaactatgaagacaagaacatcatcgtcactagacattatgagagaaccaatggtggagaaggaggagttagtggaagagaatgagtacaagaggacatgtaggagctggggatttgagaacttggagatagagaacaggagaaacaaaaatagtagtactatggcaactacttttaataaaatcattgacaatgtaaccctcgagctttttcctctccatcctgaagggagatga

SEQ ID NO: 87 - protein, 251 aa, Arabidopsis thaliana
MKVHEFSNGFSSSWDQHDSTSSLSLSCKRLRPLAPKLSGSPPSPPSSSSGVTSATFDLKNFIRPDQTGPTKFEHKRDPPHQLETHPGGTRWNPTQEQIGILEMLYKGGMRTPNAQQIEHITLQLGKYGKIEGKNVFYWFQNHKARERQKQKRNNLISLSCQSSFTTTGVFNPSVTMKTRTSSSLDIMREPMVEKEELVEENEYKRTCRSWGFENLEIENRRNKNSSTMATTFNKIIDNVTLELFPLHPEGR

SEQ ID NO: 88 - DNA, 774 bp, Brassica rapa
atgaaggttcatgagttttccaatgggtcttcgtcctgggaacaacatgattcgccatcatcccttagcctaagctgcaaacgcctccgtcctctcgcccctaagctctccggcagcccttgctctccttcttcttcctccggcgtcacttccgctactttcgacctcaagagcttcattaaacccgatcaaaccggtccaagaaaatttgaatactctattgaacaccaacgagaccttcctcaagtggagacgcaccagggagggacaaggtggaacccaactcaagaacagatagggatacttgagatgttatacaaaggtggaatgcgcactcctaatgcgcaacagatcgagcacatcacttctcaactcggtaaatacggaaaaattgaaggaaagaatgtattttactggtttcagaaccacaaagcccgcgagaggcagaagcagaagcggaacaacttcatcagcttaagttgccaaagcagcttcaagaccactaatatcaataacgcaagtgtaacaacgaagacaacaacaacaacatcgtcatttgacgtaatcaggagagactcaatggttgagaagggggagttagtggaagaaactgagtacaagaggacaggtaggagctggggatttgagaacttggagatagatagcagacgaaacataaatagtagtaaaaatgctacaatggcaactactttcgacaaaatcattgataatgtaacgctcgagcttttcccattgtaccctgaaggtaggtga

SEQ ID NO: 89 - protein, 257 aa, Brassica rapa
MKVHEFSNGSSSWEQHDSPSSLSLSCKRLRPLAPKLSGSPCSPSSSSGVTSATFDLKSFIKPDQTGPRKFEYSIEHQRDLPQVETHQGGTRWNPTQEQIGILEMLYKGGMRTPNAQQIEHITSQLGKYGKIEGKNVFYWFQNHKARERQKQKRNNFISLSCQSSFKTTNINNASVTTKTTTTTSSFDVIRRDSMVEKGELVEETEYKRTGRSWGFENLEIDSRRNINSSKNATMATTFDKIIDNVTLELFPLYPEGR

SEQ ID NO: 90 - DNA, 756 bp, Brassica rapa
atgaaggttcatgagttttccaatgggttttcgtcttgggagcagcaacaagagtcgccatcatcctttagctgcaaacgcttccgtcctctggcccctaagctctccggcagcccttcctcccctccttcttcttcctcgggcgtcacttccgctactttcgatctcaagagcttcattaaacccgatcaagccgctccaacaaaatctcactactctcttgaacacaaacgagacttttctcaagtggagatgcacccgggagggacaaggtggaacccaactcaagaacagataaaaatacttgagatcttgtacaaaggtggaatgcggactcctaacgcgcaacagatcgagcacataacttctcaactcggtaaatacggaaaaatcgaagggaagaatgtgttctactggtttcagaaccacaaagcgcgggagagacagaagcagaagaggaacaacctcagcttaagttgccaaggcagcctcagtactactagtgtctctaatgcaagtgtaacaatgaagacaagaacatcgtcttcacctgacttcaagagagaaccaatggtgatgaaggagttacttgaagagaatgagtacaagaggacatgtaggagctggggatttgagaacttgaagatagagagcagaagaaacataaatagtagtataaatgctacaatagcaactacttttaatattgacaatgtaactcttgagctttttcctttgcaccctgaaggaaggtga

SEQ ID NO: 91 - protein, 251 aa, Brassica rapa
MKVHEFSNGFSSWEQQQESPSSFSCKRFRPLAPKLSGSPSSPPSSSSGVTSATFDLKSFIKPDQAAPTKSHYSLEHKRDFSQVEMHPGGTRWNPTQEQIKILEILYKGGMRTPNAQQIEHITSQLGKYGKIEGKNVFYWFQNHKARERQKQKRNNLSLSCQGSLSTTSVSNASVTMKTRTSSSPDFKREPMVMKELLEENEYKRTCRSWGFENLKIESRRNINSSINATIATTFNIDNVTLELFPLHPEGR

SEQ ID NO: 92 - DNA, 711 bp, Brachypodium distachyon
atgaggcttcagcatctgcatgtggcctacctggagaacaagaaagcggcctccagctcttcctctgcgttgtcgccgtctcaaacaccatccaccttcccctttgccttccaatgtctccgcccgctggcgcccaagatctcgctgccggagccgaggacgaaggcgcccgagttcggtcgcgtcagaaccgccgcctccaagctgctggcatgcactgtgcaggtggcggcgggggggacgacgcggtggaacccgtccacggagcagataaaggtgctggaggcgctgtaccggggcgggatgcgaaccccgaacgcggcgcagatcgagcgcatcacggaggagctcggcaggcacggccggatcgaagggaagaacgtcttctactggttccagaaccacaaggcccgcgagcgccagaagcagaagcgcgccgcgctcctcaccctcgccacggccggcaccggcctcgacgactcttccgattccccgccgccgccgccggaaacgacgacgaccaccaaggatggagcggagaagatggaagcggcagcgtgtgccgatgtcgacgtcacgacgagctgcaagcggcggtgcaaagcgtggggtgacggcggacatggcggtgcggcggagacggagggcggcggcgccgccgacgatgtcaccttggagctcttcccgttgcgtccgcaggggaaagcttag

SEQ ID NO: 93 - protein, 236 aa, Brachypodium distachyon
MRLQHLHVAYLENKKAASSSSSALSPSQTPSTFPFAFQCLRPLAPKISLPEPRTKAPEFGRVRTAASKLLACTVQVAAGGTTRWNPSTEQIKVLEALYRGGMRTPNAAQIERITEELGRHGRIEGKNVFYWFQNHKARERQKQKRAALLTLATAGTGLDDSSDSPPPPPETTTTTKDGAEKMEAAACADVDVTTSCKRRCKAWGDGGHGGAAETEGGGAADDVTLELFPLRPQGKA

SEQ ID NO: 94 - DNA, 711 bp, Oryza sativa
atgaggcttcaccatctgcatgtggcctacctggatcacaaagcgtcgtcgtcctcgtcgtctccggcgccaccatccatctccccgtcgtcgatccccggatcggccgccttccctgctttctccttcaaatgcctccgcccgctcgcgcccaagatctcgctgccggagccgaggaagatgatcgcgccgccggacttcgtcgtccctcgcgccagaaacgcttccaagctgctcaactacactgtgcaggtgccggcggcggggacgacgaggtggaacccgtcggcggagcagataaaggtgctggagatgctgtaccgcggcgggatgcggacgccgaactccgtccagatcgagcggatcacggaggagctcggcaagtacggccggatcgagggcaagaacgtcttctactggttccagaatcacaaggcccgcgagcgccagaagcagaagcgcgccgccctcctcaccctcagcacgctcgacccttccttgctgcccgctaccgctaacgaaaccaaagaggcaccggagaagaaggagaaggacgtggaagatggattggcgagctgcaagcggcggtgcaaggcgtggggtgacggcgccggcgatggagacgcggtggtggcgacggaggcggccggcggctgcaccgacgaagtgaccctggagctcttcccgttgcatccccagggaaaagcttaa

SEQ ID NO: 95 - protein, 236 aa, Oryza sativa
MRLHHLHVAYLDHKASSSSSSPAPPSISPSSIPGSAAFPAFSFKCLRPLAPKISLPEPRKMIAPPDFVVPRARNASKLLNYTVQVPAAGTTRWNPSAEQIKVLEMLYRGGMRTPNSVQIERITEELGKYGRIEGKNVFYWFQNHKARERQKQKRAALLTLSTLDPSLLPATANETKEAPEKKEKDVEDGLASCKRRCKAWGDGAGDGDAVVATEAAGGCTDEVTLELFPLHPQGKA

SEQ ID NO: 96 - DNA, 711 bp, Medicago truncatula
atgaaggtgcatcaattcacacgtggattttgggagcatgaaccttccctcacacttggttgcaagcgtttacgtcctcttgcacctaaaatttcttctaataacaccgataattctactactaacaccccttctctttcttttgatctcaagagtttcattagacccgaaactgctcccagaattagtctttcttcctctgatgataacaagaaagatccaccttcaccacaaagccaggttgaaacgcatattccaggagggacaagatggaatccaacacaagaacaaataggaatattggagatgttatacagaggtggaatgagaactccaaatgcacaacaaatagaacagattacagttcaacttagcaagtacggtaaaattgaagggaaaaatgtgttctattggttccaaaaccacaaagcacgcgagagacaaaagcagaaacgtaacagtcttggccttcctcatagtcctcgaactcccaccaccacacttgtgtctatgtcccccacctttagtactattacaactttggacaccccaaaaagggtgcaagtaatggaaagagatcaagaagatagcccattgaagaagagtaggagttggccatttgagtatttggaagagaaaaattggtcaatgtgcaaagtagaggaacataaaactctagagcttttcccattacacccagaaggcagatga

SEQ ID NO: 97 - protein, 236 aa, Medicago truncatula
MKVHQFTRGFWEHEPSLTLGCKRLRPLAPKISSNNTDNSTTNTPSLSFDLKSFIRPETAPRISLSSSDDNKKDPPSPQSQVETHIPGGTRWNPTQEQIGILEMLYRGGMRTPNAQQIEQITVQLSKYGKIEGKNVFYWFQNHKARERQKQKRNSLGLPHSPRTPTTTLVSMSPTFSTITTLDTPKRVQVMERDQEDSPLKKSRSWPFEYLEEKNWSMCKVEEHKTLELFPLHPEGR

SEQ ID NO: 98 - DNA, 687 bp, Medicago truncatula
atgaaggtgcatcaattcacacgtggattttgggagcatgaaccttccctcacacttggttgcaagcgtttacgtcctcttgcacctaaaatttcttctaataacaccgataattctactactaacaccccttctctttcttttgatctcaagagtttcattagacccgaaactgctcccagaattagtctttcttcctctgatgataacaagaaagatccaccttcaccacaaagccaggttgaaacgcatattccaggagggacaagatggaatccaacacaagaacaaataggaatattggagatgttatacagaggtggaatgagaactccaaatgcacaacaaatagaacagattacagttcaacttagcaagtacggtaaaattgaagggaaaaatgtgttctattggttccaaaaccacaaagcacgcgagagacaaaagcagaaacgtaacagtcttggccttcctcatagtcctcgaactcccaccaccacacttgtgtctatgtcccccacctttagtactattacaactttggacaccccaaaaagggtatgttcgtattcaaataatattcaactcaacctatatatcgtggtttcgaaatgtcgttgcagataccttaaaatcctttatattgtcgacgcaactatagtcttggaccataatttaaaaccatga

SEQ ID NO: 99 - protein, 228 aa, Medicago truncatula
MKVHQFTRGFWEHEPSLTLGCKRLRPLAPKISSNNTDNSTTNTPSLSFDLKSFIRPETAPRISLSSSDDNKKDPPSPQSQVETHIPGGTRWNPTQEQIGILEMLYRGGMRTPNAQQIEQITVQLSKYGKIEGKNVFYWFQNHKARERQKQKRNSLGLPHSPRTPTTTLVSMSPTFSTITTLDTPKRVCSYSNNIQLNLYIVVSKCRCRYLKILYIVDATIVLDHNLKP

SEQ ID NO: 100 - DNA, 705 bp, Phseolus vulgaris
atgaaggtgcatcagttggcacgtggattctgggagcatgaaccctccctcacacttgggtgcaaacgcttacgcccccttgcccccaagctctccaacaccaactccactacctctatcacttcacctcctgtcacaactttcgacctcaagagcttcatcaaaccagaaagtgcctccataaaacttggatcctgtgaccacaataagagagaccctacttcacctgagggccaggtggaaacgcatattccaggagggacacggtggaatccgactcaggaacagatagggatactggagatgctgtatagaggagggatgcgaacaccgaatgctcaacaaatagagcacatcacagtccaacttagcaagtacggcaagatcgaagggaagaacgtgttctattggttccaaaaccacaaggcacgcgagagacaaaagcagaagcgtaacaacctaggccttgctcatagtcctcgtactcccaccacactactgtcatcctcctttagttgtagtgtgataacctccttcgacaccacaaaacggggggagggagtagaaagagatgaagaagatagccctttgaagaagtgtaggagctgggcatttgagtacttggaagaccaaagttggtcatcgtggaaagaggaggaacacagaactctggagcttttcccattgcacccggaaggcagatga

SEQ ID NO: 101 - protein, 234 aa, Phseolus vulgaris
MKVHQLARGFWEHEPSLTLGCKRLRPLAPKLSNTNSTTSITSPPVTTFDLKSFIKPESASIKLGSCDHNKRDPTSPEGQVETHIPGGTRWNPTQEQIGILEMLYRGGMRTPNAQQIEHITVQLSKYGKIEGKNVFYWFQNHKARERQKQKRNNLGLAHSPRTPTTLLSSSFSCSVITSFDTTKRGEGVERDEEDSPLKKCRSWAFEYLEDQSWSSWKEEEHRTLELFPLHPEGR

SEQ ID NO: 102 - DNA, 708 bp, Phseolus vulgaris
atgaccaccagcatgaaggtgcatcacttcacacgtgcattctgggagcacgaacctttcctcacccttggctgcaaccgattacgccctcttgctcccaagcttcccagcctccacaccaccaccaccccttctcttcctttcgatctcaagagcttcatcagaccagaaagtggtcccagaaaactagtttccttaatccacaataacaaggatccaccttcaccccaagcccaggttgaaacgcttccaggagggacgcggtggaatccgacgcaagaacagataggtatattggagatgctgtacaaaggagggacgagaactccaaatgctcagcagatagagcaaatcacggttcagctaggaaagtacggaaagattgaagggaagaatgtgttctattggtttcagaatcacaaagcacgcgagagacaaaagcagaagcgtaacagccttgccttttctcatagtcctcgaactcccacaattcatactgttgttactttagagacagcaaggggggaagtagtggacagagatgaggaggaaggtagtccgtgtaaaaagaagtgtaggagatgggtatttgattgtttggaagagcaaagcatgtcatcatcatcactgtgtgaagaggaggaacatagaactctggagctttttccattgcagccggaaggaagattaaggggtttgagaaactga

SEQ ID NO: 103 - protein, 235 aa, Phseolus vulgaris
MTTSMKVHHFTRAFWEHEPFLTLGCNRLRPLAPKLPSLHTTTTPSLPFDLKSFIRPESGPRKLVSLIHNNKDPPSPQAQVETLPGGTRWNPTQEQIGILEMLYKGGTRTPNAQQIEQITVQLGKYGKIEGKNVFYWFQNHKARERQKQKRNSLAFSHSPRTPTIHTVVTLETARGEVVDRDEEEGSPCKKKCRRWVFDCLEEQSMSSSSLCEEEEHRTLELFPLQPEGRLRGLRN

SEQ ID NO: 104 - DNA, 738 bp, Sorghum bicolor
atgaggcttcaccatctccatgtggcttacttggacaaagcggcggcaggctcgccgccgccgtcgtctccaccatccatctcactagtagcatctcagagctcatctgctgccatccctctggccctccagcaatattgtctgcgcccgcttgcgcccaagatctccttccctgaggcgaggaagatggtcgtccttcctgagtttgctcgcgtcaggaatgcttcttcgaggctgctaaactgcacggtgcaagtgccgacggcgggtggcacgacgcggtggaacccgtcgccggatcagataagggtgctggagatgctttaccgcggggggatgcgcacgcccaactcgttccagatcgagcagatcacggaggagctcggcaagtacggccgaatcgagggcaagaacgtcttctactggttccagaaccacaaagcccgcgagcgccagaagcagaagcgagctgccctcctcacccttagcaccaccgccacttccacgctgctaccattatcaccagcagctgctgaaaccaaagatggagttgagacgaaaaaggaagaagcgtgtgaagatgcatcgagccgcaagcggaggtgcaggacctgggatggcgatgtcgtccatggtggcggcggcgacgatgccgctaaggaggtagctgacgactactacaccgatgatgtgaccctggagctcttcccgttgcgtcctcaagggaaagctagctag

SEQ ID NO: 105 - protein, 245 aa, Sorghum bicolor
MRLHHLHVAYLDKAAAGSPPPSSPPSISLVASQSSSAAIPLALQQYCLRPLAPKISFPEARKMVVLPEFARVRNASSRLLNCTVQVPTAGGTTRWNPSPDQIRVLEMLYRGGMRTPNSFQIEQITEELGKYGRIEGKNVFYWFQNHKARERQKQKRAALLTLSTTATSTLLPLSPAAAETKDGVETKKEEACEDASSRKRRCRTWDGDVVHGGGGDDAAKEVADDYYTDDVTLELFPLRPQGKAS

SEQ ID NO: 106 - DNA, 585 bp, Triticum aestivum
  misc_feature (84)..(84): n is a, c, g, or t
atgcctccggccgctggcgcccaagatctccctcccggaccagccgaagaagctcgtcgcgccgccggacgtcctcggccgcgngtcaggaacgccaccaagctgctctgctgcaccgtcagaaaccataccgtgcaagtgccggtgggagggacgacgcggtggaacccgtcggcggagcagatcaaggtgctggaggcgctgtaccgtggcggtatgcgcaccccgaactcggcccagatcgagcgcatcacggaggagctcggccggcatggccggatcgagggcaagaacgtcttctactggttccagaaccacaaggcccgggagcggcagaagcagaagcgcgccgccctcctcaccctcagcaccctcgactcttcctccctgcctgcaacggcgaccaaggatggagccggtgataagaaggaagcttgcgacgatgccgtgacgagctgcaagcggcagtgcaggacgtggggcgatgggcatggcgatgctgcggcggaggtggccgccgagggctgcacggagaatgtcaccctggagctcttcccgttgcgtccgcaggggaaagctgcgtag

SEQ ID NO: 107 - protein, 194 aa, Triticum aestivum
MPPAAGAQDLPPGPAEEARRAAGRPRPRVRNATKLLCCTVRNHTVQVPVGGTTRWNPSAEQIKVLEALYRGGMRTPNSAQIERITEELGRHGRIEGKNVFYWFQNHKARERQKQKRAALLTLSTLDSSSLPATATKDGAGDKKEACDDAVTSCKRQCRTWGDGHGDAAAEVAAEGCTENVTLELFPLRPQGKAA

SEQ ID NO: 108 - DNA, 714 bp, Triticum aestivum
atgagggttcaccatctgcatgtgccctcctacctggagaaagcggcctcggcgtcttctccgtcgccggctacggcaccgtccacctctcctccctcggccctgttcccttacggggccttccagtgcctccgcccgctcgcgcccaagatctcgctcccggaccagccgaggaagctggtcgcgccgcccgacgtcctcggccgcgtcaggaacgccaccaagctgctcagttgcaccgtcagaaaccacaccgtgcaagtgccggtgggagggacgacgcggtggaacccgtcggcggagcagatcaaggtgctggaggcgctgtaccgcggcgggatgcgcaccccgaactcggcccagatcgagcgcatcacggaggagctcggcaggcacggccggatcgagggcaagaacgtcttctactggttccagaaccacaaggcccgggagcggcagaagcagaagcgcgccgccctcctcaccctcagcaccctcgactcttcctccccgcctgcaacggcgacgaccaaggatggagcaggtgatgatgagaaggaagcttgcgacgatgagacgatgaggtgcaagcggcggtgcaggacgtggggcgatgggcatggcgacgcggtggcggaggtggccgccgacggctgcacggataacgttaccctggagctcttcccgttgcgtccgcaggggaaagctgcgtag

SEQ ID NO: 109 - protein, 237 aa, Triticum aestivum
MRVHHLHVPSYLEKAASASSPSPATAPSTSPPSALFPYGAFQCLRPLAPKISLPDQPRKLVAPPDVLGRVRNATKLLSCTVRNHTVQVPVGGTTRWNPSAEQIKVLEALYRGGMRTPNSAQIERITEELGRHGRIEGKNVFYWFQNHKARERQKQKRAALLTLSTLDSSSPPATATTKDGAGDDEKEACDDETMRCKRRCRTWGDGHGDAVAEVAADGCTDNVTLELFPLRPQGKAA

SEQ ID NO: 110 - DNA, 747 bp, Triticum aestivum
  misc_feature (201)..(201): n is a, c, g, or t
atgagggttcaccatttgcatgcgccgtcctacctggagaaagcggcctcggcgtcttcgtctccggctactgcaccgtccacgtctcctccctcggccctgttccctttcggcgccttccaatgcctccggccgctggcgcccaagatctccctcccggaccagccgaagaagctcgtcgcgccgcccgacgtcctccgnaagaagctcgtcgcgccgcccgacgtcctcggccgcgtcaggaacgccaccaagctgctcagctgcaccgtcagaaaccatacagtgcaagtgcaagtgccggtgggagggacgacgcggtggaacccgtcggcggagcagatcaaggtgctggaggcgctgtaccgcggcgggatgcgcaccccgaactcggcccagatcgagcgcatcacggaggagctcggccggcacggccggatcgaaggcaagaacgtcttctactggttccagaaccacaaggcacgggagcggcagaagcagaagcgcgccgccctcctcaccctcagcaccctcgactcttcctccctgcctgcaacggcgaccaaggatggagccggtgataagaaggaagattgcgatgaggcgacgaacagctgcaaacggcggtgcatgacatggagtgccggccatggcgacgcggcggcggaggtggccgcccccgacggctgcacggataacgtcaccctggagctcttcccgttgcgtccacaggggaaagctgcgtag

SEQ ID NO: 111 - protein, 248 aa, Triticum aestivum
MRVHHLHAPSYLEKAASASSSPATAPSTSPPSALFPFGAFQCLRPLAPKISLPDQPKKLVAPPDVLRKKLVAPPDVLGRVRNATKLLSCTVRNHTVQVQVPVGGTTRWNPSAEQIKVLEALYRGGMRTPNSAQIERITEELGRHGRIEGKNVFYWFQNHKARERQKQKRAALLTLSTLDSSSLPATATKDGAGDKKEDCDEATNSCKRRCMTWSAGHGDAAAEVAAPDGCTDNVTLELFPLRPQGKAA

SEQ ID NO: 112 - DNA, 549 bp, Arabidopsis thaliana
atgtctttctccgtgaaaggtcgaagcttacgtggcaacaataacggaggaacggggacgaagtgcgggagatggaatccaacggtggagcagttgaagatattgactgatctgtttcgagccggtcttagaactccaacaactgatcagattcagaagatctctacggagctcagtttctacggcaagatagagagcaagaatgttttctattggtttcagaatcataaggctagggagaggcagaaacgtcgtaaaatctccattgattttgatcatcatcatcatcaaccatcaactagagatgtttttgaaataagcgaagaagattgtcaagaggaagagaaggtgatagagacattacaactctttccggtgaattcatttgaagactccaactccaaggtggacaaaatgagagctagaggcaataaccagtaccgtgaatatattcgagagaccaccacgacgtcgttttctccatactcatcatgtggagctgaaatggaacatccaccgccattagatcttcgattaagctttctttaa

SEQ ID NO: 113 - protein, 182 aa, Arabidopsis thaliana
MSFSVKGRSLRGNNNGGTGTKCGRWNPTVEQLKILTDLFRAGLRTPTTDQIQKISTELSFYGKIESKNVFYWFQNHKARERQKRRKISIDFDHHHHQPSTRDVFEISEEDCQEEEKVIETLQLFPVNSFEDSNSKVDKMRARGNNQYREYIRETTTTSFSPYSSCGAEMEHPPPLDLRLSFL

SEQ ID NO: 114 - DNA, 369 bp, Arabidopsis thaliana
atgtcgtcgagaggattcaacattaaagctagaggattatgtaataacaacaacggaggaggaggaacgggggcgaagtgtggacggtggaatccaacggtggagcaagtgaagcttctgacagatctgttcaaggcgggactgcgaacaccgagcacggaccagattcagaagatctctatggagctgagtttctacggtaagattgagagcaagaacgtgttctattggttccaaaaccataaagctagagagagacaaaagtgccggaaaatctccaccgtcaagtttgatcatcgtcaagatacagatctttctaagcctcgccgagacaacgtacgtcgtcatcaactaccagcgaaaggttaa

SEQ ID NO: 115 - protein, 122 aa, Arabidopsis thaliana
MSSRGFNIKARGLCNNNNGGGGTGAKCGRWNPTVEQVKLLTDLFKAGLRTPSTDQIQKISMELSFYGKIESKNVFYWFQNHKARERQKCRKISTVKFDHRQDTDLSKPRRDNVRRHQLPAKG

SEQ ID NO: 116 - DNA, 579 bp, Brassica rapa
atgacgtcaggagaattctccattaaagctagagggttaggtgataacaacaacggaggtggaggaacagggacaaagtgtggaaggtggaatccaacggtggagcaagtgaagcttctgacggacctgttcaaggcggggctacgaacaccgagcaccgatcagattcagaagatctctatggagctgagtttctacggtaagatcgaaagcaagaacgtgttttattggttccaaaaccataaagctagggaaagacaaaagcgccgtaaaatctccaccgtcgactttgatcatcgtcaagaaacaaatctctctattcctcatcgacacaacatacgtcatcatcaaccaccacctaaagatacttttgaagcttgtgaaaaagaggagaaggtgatagagacgttgcaactcttcccattgtcgaatgttgaaagagcgagaacaaacattactgctacgagccacaacgaatacatacgagagcatgtcaatacgacggtgttttctacctactcatcgtgtggagctgagatggaacatccgccgttggatcttcgtttgagctttttatga

SEQ ID NO: 117 - protein, 192 aa, Brassica rapa
MTSGEFSIKARGLGDNNNGGGGTGTKCGRWNPTVEQVKLLTDLFKAGLRTPSTDQIQKISMELSFYGKIESKNVFYWFQNHKARERQKRRKISTVDFDHRQETNLSIPHRHNIRHHQPPPKDTFEACEKEEKVIETLQLFPLSNVERARTNITATSHNEYIREHVNTTVFSTYSSCGAEMEHPPLDLRLSFL

SEQ ID NO: 118 - DNA, 525 bp, Brassica rapa
atgggaggtggaggaacagggactaagtgtggacggtggaatccaacggtggagcaagtgaaacttctgacggatctgttcaaggcggggctacgaacaccaaacaccgaccagattcaaaaaatctctacggagctgagtgcctacggtaagatcgagagcaagaacgtgttctattggttccaaaaccataaagctagggaaagacaaaagcgccgtaaaatctccaccgtcgattacgatcatcgtcaagacacaaatctctctctttcttatcgacacaacccatgtcatcaccaaacaccacctaaagatacttttgaaggccgtgaagaagaggagaatgtgatagagacattgcaactcttcccattatcgaaggttgaaagagctaatattactgctgccagctacaacgaatacataagagagcatgtcaatacgacggtgttttccacatactcatcatcatgcggagctgagatggaacatccgccgttggatcttcgactaagctttctatga

SEQ ID NO: 119 - protein, 174 aa, Brassica rapa
MGGGGTGTKCGRWNPTVEQVKLLTDLFKAGLRTPNTDQIQKISTELSAYGKIESKNVFYWFQNHKARERQKRRKISTVDYDHRQDTNLSLSYRHNPCHHQTPPKDTFEGREEEENVIETLQLFPLSKVERANITAASYNEYIREHVNTTVFSTYSSSCGAEMEHPPLDLRLSFL

SEQ ID NO: 120 - DNA, 432 bp, Brassica rapa
atgtctttccccctgaaagttcgtagcgtacgtggcaacaacggaggaggaggagggacggggacgaagtgcgggaggtggaacccgacggtggagcagttgaaggtattgactgacttgttccgagctggtcttagaactccgaccacagaccaaattcagaagatctctacagagctcagtttctacggcaagatcgagaatgtttttgaaataaacgaagacgaggaggagagggtaatagagacgctacaactctttccggtgaattcattccaagaatccaaagtggaaaaaatgagaagcagagtcaataaccagtaccgtgaatacatacgagagaccaacacgaccacgtttccttcctactcatcatgtggagctgagatggaacatccatcgccgttggaacttcgattaagctttctttga

SEQ ID NO: 121 - protein, 143 aa, Brassica rapa
MSFPLKVRSVRGNNGGGGGTGTKCGRWNPTVEQLKVLTDLFRAGLRTPTTDQIQKISTELSFYGKIENVFEINEDEEERVIETLQLFPVNSFQESKVEKMRSRVNNQYREYIRETNTTTFPSYSSCGAEMEHPSPLELRLSFL

SEQ ID NO: 122 - DNA, 636 bp, Brachypodium distachyon
atggaggtgctgagcgggagggtaggggtgaagtgcgggcggtggaacccgacggcggagcaggtgaaggtgctgacggagcttttccgggcggggctgcggacgccgagcacggagcagatccagcggatctccacccacctcggcgccttcggcaaggtggagagcaagaacgtcttctactggttccagaaccacaaggcccgcgagcgccaccaccacaagaagcgccgccgcgtcgcctcctcctcctccgacaacagcagcgccagcaacaacaacgacgaagcagcagatcacggccgcagtagtgcccgcgaggacctcctgctgcagcctcccgagagcaagcgcgaggccagaagctacaaccaccaccgccggccgatcatgacatatgtaggctatgtgagggacgaggtggagcaggaggtggtgatgtgggagcggccgacgagggaggtggagacgctcgagctgttcccgctcaaggcagcctacgacctcgaggcggcggacaggctccggtacgtgaggggcgccggcgagcagcagtgcagggagatctccttcttcgacgtcgccaacggacgggatccgccgctggagctcaggctctgcagcttcgatatctag

SEQ ID NO: 123 - protein, 211 aa, Brachypodium distachyon
MEVLSGRVGVKCGRWNPTAEQVKVLTELFRAGLRTPSTEQIQRISTHLGAFGKVESKNVFYWFQNHKARERHHHKKRRRVASSSSDNSSASNNNDEAADHGRSSAREDLLLQPPESKREARSYNHHRRPIMTYVGYVRDEVEQEVVMWERPTREVETLELFPLKAAYDLEAADRLRYVRGAGEQQCREISFFDVANGRDPPLELRLCSFDI

SEQ ID NO: 124 - DNA, 666 bp, Zea mays
atggaggcgctgagcgggcgggtaggcgtcaagtgcgggcggtggaaccctacggcggagcaggtgaaggtcctgacggagctcttccgcgcggggctgcggacgcccagcacggagcagatccagcgcatctccacccacctcagcgccttcggcaaggtggagagcaagaacgtcttctactggttccagaaccacaaggcccgcgagcgccaccaccacaagaagcgacgccgcggcgcgtcgtcgtcctcccccgacagcggcagcggcaggggaagcaacaacgaggaagacggccgtggtgccgcctcgcagtcgcacgacgccgacgccgacgccgacctcgtgctgcaaccgccagagagcaagcgggaggccagaagctatggccaccatcaccggctcgtgacatgctacgtcagggacgtggtggagcagcaggaggcgtcgccgtcgtgggagcggccgacgagggaggtggagacgctagagctcttccccctcaagtcgtacggcgacctcgaggcggcggagaaggtccggtcgtacgtcagaggaagcggcgccaccagcgagcagtgcagggagttgtccttcttcgacgtcgtctccgccggccgggatccgccgctcgagctcaggctctgcagcttcggtccctag

SEQ ID NO: 125 - protein, 221 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADADADLVLQPPESKREARSYGHHHRLVTCYVRDVVEQQEASPSWERPTREVETLELFPLKSYGDLEAAEKVRSYVRGSGATSEQCRELSFFDVVSAGRDPPLELRLCSFGP

SEQ ID NO: 126 - DNA, 708 bp, Zea mays
atggacacggaggcgccgagcgggcgggtgggcgtcaagtgcgggcggtggaacccgacgccggagcaggtgaaggtcctgacggagctcttccgcgcggggctgcggacgcccagcacggaccagatccagcgcatctccgcccacctcggcgccttcggcaaggtggagagcaagaacgtcttctactggttccagaaccacaaggcccgcgagcgccaccaccacaagaagaagaagcgccgccgacgcggcgcggcaatggcatcgtcgtcctcccccgacagcggcagcggcagcgggagcagcgaggaagacggccgtgttgccgccgccgccgacgccgacctcgtgctgcaaccatcgccgggaagaagcaaaagcaaacgggaggccagaagaagctacggcggcggccaccatcgccggctcgttacatgtaagcagctagcgcacacacatcgctacgtcagggacgttgtggagcagcaggaggaggacacgtgggagcggccgacgagggaggcggtggagactctggagctcttccccctcaagtcgtacgtcgacctcgaggcggagaaggtccggcggtacgtcgtcagggccggcgccagcgagcagtgcagggacttctccttcttcgacgtgtccggcggtcgggatccgccgcttcttgagcttaggctctgcagcttcggtccctag

SEQ ID NO: 127 - protein, 235 aa, Zea mays
MDTEAPSGRVGVKCGRWNPTPEQVKVLTELFRAGLRTPSTDQIQRISAHLGAFGKVESKNVFYWFQNHKARERHHHKKKKRRRRGAAMASSSSPDSGSGSGSSEEDGRVAAAADADLVLQPSPGRSKSKREARRSYGGGHHRRLVTCKQLAHTHRYVRDVVEQQEEDTWERPTREAVETLELFPLKSYVDLEAEKVRRYVVRAGASEQCRDFSFFDVSGGRDPPLLELRLCSFGP

SEQ ID NO: 128 - DNA, 603 bp, Oryza sativa
atggaggctcttagcgggcgagtgggggtgaagtgtgggcggtggaacccgacggcggagcaggtgaaggtcctgacggagctgttccgagcggggttgcggacgccgagcacggagcagatccagcgcatctccactcacctcagcgcattcggcaaggtggagagcaagaacgtcttctactggttccagaaccacaaggcccgcgagcgccaccaccacaagaagcgccgccgcggcgcctcctcccccgacagcggcagcaacgatgacgatggccgcgccgcagcccacgagggcgacgccgacctcgtcctgcagcctcctgagagcaagcgggaggccagaagctacggccatcatcaccggctcatgacatgctacgtgagggatgtggtggagacggaagcgatgtgggagcggccgacgagggaggtggaaacgctggagctgttcccactcaagtcgtatgacctcgaggtggacaaggtccggtatgtgaggggcggcggtggcgagcagtgcagggagatctcgttcttcgacgtcgctgccggccgggatccgccgctggagctcaggctttgcagcttcggcctctag

SEQ ID NO: 129 - protein, 200 aa, Oryza sativa
MEALSGRVGVKCGRWNPTAEQVKVLTELFRAGLRTPSTEQIQRISTHLSAFGKVESKNVFYWFQNHKARERHHHKKRRRGASSPDSGSNDDDGRAAAHEGDADLVLQPPESKREARSYGHHHRLMTCYVRDVVETEAMWERPTREVETLELFPLKSYDLEVDKVRYVRGGGGEQCREISFFDVAAGRDPPLELRLCSFGL

SEQ ID NO: 130 - DNA, 555 bp, Medicago truncatula
atggaagagagcatgtcaggcttttgcatgaaatcatctggaagtagtaatttccatggtaaaagtggaagcagcagcactggcacaaagtgtggtcgttggaatccaacaacagaacaagttaaacttctgactgaacttttcagagcagggctcagaactccaagcactgatcaaattcagaaaatatccaatcaactcagtttttatggtaagatagagagcaagaatgtgttctattggtttcaaaatcacaaagctagagaaagacaaaaacgtcgtaaggtttcttttgatgatgacaaggatgtcatagttcatagagacaactctatgaatgcttcaacacaaaggtttgctgagatgtactcagagcctgatagagtgattgagactcttgagctttttccattgaactcatttggtgaatcagaatcagaaaagtggagtatgcatgcaaatgaatgcagggaaaatacaatgtttgcatacacatgtacaatgggagaacaaatggaacacccaccattggacttacggttgagttttgtgtaa

SEQ ID NO: 131 - protein, 184 aa, Medicago truncatula
MEESMSGFCMKSSGSSNFHGKSGSSSTGTKCGRWNPTTEQVKLLTELFRAGLRTPSTDQIQKISNQLSFYGKIESKNVFYWFQNHKARERQKRRKVSFDDDKDVIVHRDNSMNASTQRFAEMYSEPDRVIETLELFPLNSFGESESEKWSMHANECRENTMFAYTCTMGEQMEHPPLDLRLSFV

SEQ ID NO: 132 - DNA, 534 bp, Phseolus vulgaris
atggaagagggcatgtcagatttttgtttcagaggtgaaaatgttggttgtaatagaggtagtggtagtggtaccaagtgtgggcgttggaatcccacaactgagcaggctaaggttctgactcaactgttcagttctggacttcgaaacccaagcattgatcagattcaaaagatctctaatcagctcagtttttatggtaagattgagagcaaaaatgtgttctattggtttcagaaccataaggccagggaaagacaaaagcgtcgcaaggttgatagggatgtgactcgtagtgaaaactctatctccattaattttttcacacaaaattttactcagctatatgacgtttctgagactgagaaagtcactgagacccttcaactgttccccctgaactctttttgtgaatcagaatcagagaatatgagggttcatgcaagtgactgcaggaataatacagtgttttctttcacagaggaggaacaaatgaatcattcaccattagatcttcgcttgagtttcacgtga

SEQ ID NO: 133 - protein, 177 aa, Phseolus vulgaris
MEEGMSDFCFRGENVGCNRGSGSGTKCGRWNPTTEQAKVLTQLFSSGLRNPSIDQIQKISNQLSFYGKIESKNVFYWFQNHKARERQKRRKVDRDVTRSENSISINFFTQNFTQLYDVSETEKVTETLQLFPLNSFCESESENMRVHASDCRNNTVFSFTEEEQMNHSPLDLRLSFT

SEQ ID NO: 134 - DNA, 768 bp, Phseolus vulgaris
atgaaacagatcgaatacagtacatgcaatgaaaaacattacaagattaatatatatgctatattttctatgcgcaatttgatgttgtttttctatggaaaggtattatatatatatgcgcacgattgggatctttatggttcagataatctcagaactaataaagttgagttcagaggcagagagagagagagaaaatcatatctggaagaaaacatggatgagggcatgtcaggcttttgcatcagatcaggaagtgtccgtggtaaaagtggaagcactacttcttctgccactggcaccaagtgtgggcgttggaatcccaccactgaacaggttaaacttctaactgaactcttcaggtcagggctccgcactccaagcactgaccagattcaaaagatctccactcagctcagcttttacggtaagattgagagcaagaatgtcttctattggtttcagaaccataaggccagggaaagacagaagcgccgcaaggtctcctttgatgacaaggatctcattcgtagacaaaactccttcaatacttccacacaaagtcttgctgatacgtatcgagtttcagagccagatagggtgattgagactcttcaactctttcccttgaactccttcggtgaatctgaacaagagaacctaaggctacatgcaaatgaatttagggatagtagtacaatgttttcgtacatgatggatgaacaaatagagcacccaccattggacctgcgcttgagctttctgtaa

SEQ ID NO: 135 - protein, 255 aa, Phseolus vulgaris
MKQIEYSTCNEKHYKINIYAIFSMRNLMLFFYGKVLYIYAHDWDLYGSDNLRTNKVEFRGRERERKSYLEENMDEGMSGFCIRSGSVRGKSGSTTSSATGTKCGRWNPTTEQVKLLTELFRSGLRTPSTDQIQKISTQLSFYGKIESKNVFYWFQNHKARERQKRRKVSFDDKDLIRRQNSFNTSTQSLADTYRVSEPDRVIETLQLFPLNSFGESEQENLRLHANEFRDSSTMFSYMMDEQIEHPPLDLRLSFL

SEQ ID NO: 136 - DNA, 657 bp, Sorghum bicolor
atggaggcgctgagcgggcgggtaggcgtcaagtgcgggcggtggaaccctacggcggagcaggtgaaggtcctgacggagctcttccgcgcggggctgcgcacgcccagcacggagcagatccagcgcatctccacccacctcagcgccttcggcaaggtggagagcaagaacgtcttctactggttccagaaccacaaggcccgcgagcgccaccaccacaagaagcgccgccgcggcgcgtcctcccccgacagtggcagcggcagcggcagcggcagcaacgaggaagacggcggccgtgctgctgccgcctcgcacgacgccgaggccgacgtcgacctcgtgctgcagccgccagagagcaagcgggaggccagaagctacgcccaccatcatcaccggctggccgtgacatgctacgtcagggacgtggtggagcagcaggaggcgacgtgggagcggccgacgagggaggtggagacgctggagctcttccctctcaagtcgtacgtggacctggaggcggcggagaaggtccggtatgtcaggggcagcgccgccagcgagcagtgcagggagttctccttcttcgacgtctccggcggccgggatccgccacttgagctcaggctctgcagcttcggtccctactaa

SEQ ID NO: 137 - protein, 218 aa, Sorghum bicolor
MEALSGRVGVKCGRWNPTAEQVKVLTELFRAGLRTPSTEQIQRISTHLSAFGKVESKNVFYWFQNHKARERHHHKKRRRGASSPDSGSGSGSGSNEEDGGRAAAASHDAEADVDLVLQPPESKREARSYAHHHHRLAVTCYVRDVVEQQEATWERPTREVETLELFPLKSYVDLEAAEKVRYVRGSAASEQCREFSFFDVSGGRDPPLELRLCSFGPY

SEQ ID NO: 138 - DNA, 636 bp, Triticum aestivum
atggaggcgctgagcgggcgggtgggagtgaagtgcgggcggtggaacccgacggcggagcaggtgaaggtgctgacggagctgttccgggcggggctgcggacgccgagcacggagcagatccagcggatctccacgcacctcagcgccttcggcaaggtggagagcaagaacgtcttctactggttccagaaccacaaggcccgcgagcgccaccaccacaagaagcgccgccgcgtcgcctcctgctcccccgacagcagcagcaacgaggaagagagcggccgtgccgccgccgccgagcccgcggacctcgtgctccagcctcccgagagcaagcgggaggccagaggctacaaccaccatccccggatcatgacatatgcaggctatgtgagggaggtgacggagcaggaggaggcgacgacgtgggagcggccgacgagggaggtggagacgctggagctgttcccgctcaaagcagcctacgacctggagctagaggcggacaggttcagccggtacgtgaggggcggcgagcagcagtgcagggagatctccttcttcgacgtggccactggacgagatccgccgctggagctcaggctctgcagcttcgatcggtatctggtctaa

SEQ ID NO: 139 - protein, 211 aa, Triticum aestivum
MEALSGRVGVKCGRWNPTAEQVKVLTELFRAGLRTPSTEQIQRISTHLSAFGKVESKNVFYWFQNHKARERHHHKKRRRVASCSPDSSSNEEESGRAAAAEPADLVLQPPESKREARGYNHHPRIMTYAGYVREVTEQEEATTWERPTREVETLELFPLKAAYDLELEADRFSRYVRGGEQQCREISFFDVATGRDPPLELRLCSFDRYLV

SEQ ID NO: 140 - DNA, 603 bp, Oryza sativa; identical to SEQ ID NO: 128

SEQ ID NO: 141 - protein, 200 aa, Oryza sativa; identical to SEQ ID NO: 129

SEQ ID NO: 142 - DNA, 657 bp, Sorghum bicolor; identical to SEQ ID NO: 136

SEQ ID NO: 143 - protein, 218 aa, Sorghum bicolor; identical to SEQ ID NO: 137

SEQ ID NO: 144 - DNA, 633 bp, Triticum aestivum
atggaggcgctgagcgggcgggtgggagtgaagtgcgggcggtggaacccgacggcggagcaggtgaaggtgctgacggagctgttccgggcggggctgcggacgccgagcaccgagcagatccagcggatctccacccacctcagcgccttcggcaaggtggagagcaagaacgtcttctactggttccagaaccacaaggcccgcgagcgccaccaccacaagaagcgccgccgcgtcgcatcctgctcccccgacagcagcagcaacgatgaagagaccggccgtgccgccgccgccgagcccgccgacctcgtgctccagcctcccgagagcaagcgggaggccggaggctacaaccaccacccccggatcatgacatgctatgtgagggaggtggcggagcaggaagaggcgacgacgtgggagcggccgacgagggaagtggagacgctggagctgttcccgctcaaagcagcctgctacgacctggagctggaggcggacaggttcagccggtatgtgaggagcggcgagcaacagtgcagggagatctccttcttcgacgtggccaccggacgggatccgccgctggagctcaggctctgcagcttcgatcggtatctggtctaa

SEQ ID NO: 145 - protein, 210 aa, Triticum aestivum
MEALSGRVGVKCGRWNPTAEQVKVLTELFRAGLRTPSTEQIQRISTHLSAFGKVESKNVFYWFQNHKARERHHHKKRRRVASCSPDSSSNDEETGRAAAAEPADLVLQPPESKREAGGYNHHPRIMTCYVREVAEQEEATTWERPTREVETLELFPLKAACYDLELEADRFSRYVRSGEQQCREISFFDVATGRDPPLELRLCSFDRYLV

SEQ ID NO: 146 - protein, 219 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADDADLVLQPPESKREARSYGHHHRLVTCYVRDVVEQQEASPSWERPTREVETLELFPLKSYGDLEAAEKVRSYVRGIAATSEQCRELSFFDVSAGRDPPLELRLCSFGP

SEQ ID NO: 147 - DNA, 816 bp, Arabidopsis thaliana
atgggctacatctccaacaacaacctcatcaactatttgcccctctctactactcaacctcctcttcttctcacccactgtgatattaacggcaatgatcaccatcagctcataaccgcatcatcaggagaacacgatattgatgaacggaaaaacaacattcctgcggcggcgactttgagatggaatccgacgccagagcagatcacgacgctagaagagctttacagaagcggaacacggacgccgacgacggaacagatccaacagatagcatctaagcttcgtaaatatgggagaatcgaagggaagaacgttttctattggtttcagaatcataaggctagagagagactaaaacgccgccgtcgtgaaggtggtgctattatcaaaccacataaagacgtcaaggattcatcatcaggtggtcatcgagttgatcagacaaagctctgcccatcttttccacacacaaaccgaccacagccacagcatgaattagatcctgcgagttacaataaagacaacaatgctaataatgaagatcatgggacgactgaagaatctgatcagagggcatcagaggttggtaaatacgccacatggagaaatcttgttacttggtcgataactcaacaaccggaagagattaatatcgacgaaaatgtcaacggagaagaagaagaaacgagggacaaccggactttaaatctctttccggttagggagtaccaagagaaaacaggccggttgatagagaagacgaaagcatgcaactactgttactactacgagttcatgcctctgaagaactga

SEQ ID NO: 148 - protein, 271 aa, Arabidopsis thaliana
MGYISNNNLINYLPLSTTQPPLLLTHCDINGNDHHQLITASSGEHDIDERKNNIPAAATLRWNPTPEQITTLEELYRSGTRTPTTEQIQQIASKLRKYGRIEGKNVFYWFQNHKARERLKRRRREGGAIIKPHKDVKDSSSGGHRVDQTKLCPSFPHTNRPQPQHELDPASYNKDNNANNEDHGTTEESDQRASEVGKYATWRNLVTWSITQQPEEINIDENVNGEEEETRDNRTLNLFPVREYQEKTGRLIEKTKACNYCYYYEFMPLKN

SEQ ID NO: 149 - DNA, 831 bp, Brassica rapa
atgggctacatctccaacaacaacctaatcaactatttggccttctctactactaaacctcatcttctatcacaatgcaatagtatcgacaatgatcatcatcaacagctcattaacggctcagaaaataactcggcggcggctgcgtcgtcgagatggaatcctacgccggagcagatcacagcgctggaagagatatacaaaaggggaacacggacgcctacgacagaacaaatccaacagatagcatctaagctcagtaagtatggccgaatcgagggcaaaaacgttttctattggtttcagaaccacaagtctagagagagacttaagcgacgccgttgtgaaggagataatgatattaacagcgttcatgaaccacttaaagacgtcaaggattcatcatcaggtggttatcgagttgatcagacaaagatctatgcatcttttccacacacaaaccaacgaccacagaatgaattagttctgcctaatctaatcaataatgaagatcactgtaagactgaagaatcagaaagggcatcggatgcaggcaaagaagccatgtgtggaaatcttgttgcttcattcgtaaatcaagaaccaggagagattaatatcgaagaagatagatacaatgtcaggggagaagtacaagaagaagaagaagaagatgaaacaagggagatccaaactttaaatctcttcccggtactggagaaccaagagaaaaccgactggtttgcagagaagaagaaagcaaatgtgatatcttgcatatttgcattgttttatccattcatccatgagcattttcatcatatagattag

SEQ ID NO: 150 - protein, 276 aa, Brassica rapa
MGYISNNNLINYLAFSTTKPHLLSQCNSIDNDHHQQLINGSENNSAAAASSRWNPTPEQITALEEIYKRGTRTPTTEQIQQIASKLSKYGRIEGKNVFYWFQNHKSRERLKRRRCEGDNDINSVHEPLKDVKDSSSGGYRVDQTKIYASFPHTNQRPQNELVLPNLINNEDHCKTEESERASDAGKEAMCGNLVASFVNQEPGEINIEEDRYNVRGEVQEEEEEDETREIQTLNLFPVLENQEKTDWFAEKKKANVISCIFALFYPFIHEHFHHID

SEQ ID NO: 151 - DNA, 780 bp, Brassica rapa
atgaacgacatcagaaatgatgagcagatcataaccgaatccgccggagaacccaaagagagggaaaaatacatgcattcacggtggaatccgacacctgagcagacgatggtgcttgaggaggtttacagcagcggaactcggacgccgacgacacaacagatccaagagattgcatctaagctccaaaagtatgggagaattgaaggaaagaacgttttctactggttccaaaaccataagtcaagggagaggctgaaacgacgccgtggtgatcaacaaggagttactactattagtaacgttcatgaagaaactctacgcaaggacaacgtcattgtagatacggcaaacaaggattcatcatcagtctcgagaaggagaggtgatcatcgagttattcacacaaggacttgtttatctccttcacccacacatccaccgaaaaactctattgatgatgggaagcgggaagaatataagagggggttagaggaagaaaaggaaacaacttcccaaaatcagatacatccaagcaacacttcagactttaactaccatctgataactgcttctaaaccaagtcaagaagaagagcagcagtacaagctgaatgatgatgaagaagaagaagaagaaacaaggaaaagccgaacgctcgatctctttccggttatagagaaccaagagacaatcgattttgaagagaagaacacaaaaccaaaccagttgtactgcaactactgttattattacgagttcatgcctctgatgaactga

SEQ ID NO: 152 - protein, 259 aa, Brassica rapa
MNDIRNDEQIITESAGEPKEREKYMHSRWNPTPEQTMVLEEVYSSGTRTPTTQQIQEIASKLQKYGRIEGKNVFYWFQNHKSRERLKRRRGDQQGVTTISNVHEETLRKDNVIVDTANKDSSSVSRRRGDHRVIHTRTCLSPSPTHPPKNSIDDGKREEYKRGLEEEKETTSQNQIHPSNTSDFNYHLITASKPSQEEEQQYKLNDDEEEEEETRKSRTLDLFPVIENQETIDFEEKNTKPNQLYCNYCYYYEFMPLMN

SEQ ID NO: 153 - DNA, 879 bp, Arabidopsis thaliana
atggagccgccacagcatcagcatcatcatcatcaagccgaccaagaaagcggcaacaacaacaacaacaagtccggctctggtggttacacgtgtcgccagaccagcacgaggtggacaccgacgacggagcaaatcaaaatcctcaaagaactttactacaacaatgcaatccggtcaccaacagccgatcagatccagaagatcactgcaaggctgagacagttcggaaagattgagggcaagaacgtcttttactggttccagaaccataaggctcgtgagcgtcagaagaagagattcaacggaacaaacatgaccacaccatcttcatcacccaactcggttatgatggcggctaacgatcattatcatcctctacttcaccatcatcacggtgttcccatgcagagacctgctaattccgtcaacgttaaacttaaccaagaccatcatctctatcatcataacaagccatatcccagcttcaataacgggaatttaaatcatgcaagctcaggtactgaatgtggtgttgttaatgcttctaatggctacatgagtagccatgtctatggatctatggaacaagactgttctatgaattacaacaacgtaggtggaggatgggcaaacatggatcatcattactcatctgcaccttacaacttcttcgatagagcaaagcctctgtttggtctagaaggtcatcaagaagaagaagaatgtggtggcgatgcttatctggaacatcgacgtacgcttcctctcttccctatgcacggtgaagatcacatcaacggtggtagtggtgccatctggaagtatggccaatcggaagttcgcccttgcgcttctcttgagctacgtctgaactag

SEQ ID NO: 154 - protein, 292 aa, Arabidopsis thaliana
MEPPQHQHHHHQADQESGNNNNNKSGSGGYTCRQTSTRWTPTTEQIKILKELYYNNAIRSPTADQIQKITARLRQFGKIEGKNVFYWFQNHKARERQKKRFNGTNMTTPSSSPNSVMMAANDHYHPLLHHHHGVPMQRPANSVNVKLNQDHHLYHHNKPYPSFNNGNLNHASSGTECGVVNASNGYMSSHVYGSMEQDCSMNYNNVGGGWANMDHHYSSAPYNFFDRAKPLFGLEGHQEEEECGGDAYLEHRRTLPLFPMHGEDHINGGSGAIWKYGQSEVRPCASLELRLN

SEQ ID NO: 155 - DNA, 885 bp, Brassica rapa
atggagccagcgcaacatcaccatcaagccgaccaagaaagcggcaacaacaacaagtccgtctctggtggttacacgtgtcgtcaaacgagtacaagatggacaccaacgaccgatcaaatcagaatactcaaagatctttactacaacaacggagtccggtcaccaacagccgaccagatccagaagatctctgcaaggctgagacagtacggaaagatcgagggaaaaaatgtcttttactggtttcaaaaccataaggctcgtgagcgacagaagaagagattcaacagcacaaccatgaccataccaacgtcttcatcgcccaactcggttatgatggctagtgatcactatcaccataaccatcatcatcatggcgttaccatccagagacctgctttggttaacgctaagctcgaccaagaaaatcatatgtttcatcagagcagatcatatcccagcttcaataacgggaatacaaatcatgcaagttcaggcacggaatatggtgttttcagtgcttctaatggctacattagtagccatatctatgaacctatggaacaagactgttcaatgagctacaacaacgtaggtggaggatggacaaacatagatcataatcaccattactcaactccagcctacaacttcttcgatagaccaatgcttctgtctggactagaaggtcatcatcaagaagaaaaagaatatggtggcgatgcttatctggaacatagacgcacacttcctctcttccctttgcacggtgaggagcacatcaacggcggtggtggttccatcttgaagtacggacaatcggacggttgtgatcgttatggtagaggcccttgtgcttctcttaagctgtgtctgaactga

SEQ ID NO: 156 - protein, 294 aa, Brassica rapa
MEPAQHHHQADQESGNNNKSVSGGYTCRQTSTRWTPTTDQIRILKDLYYNNGVRSPTADQIQKISARLRQYGKIEGKNVFYWFQNHKARERQKKRFNSTTMTIPTSSSPNSVMMASDHYHHNHHHHGVTIQRPALVNAKLDQENHMFHQSRSYPSFNNGNTNHASSGTEYGVFSASNGYISSHIYEPMEQDCSMSYNNVGGGWTNIDHNHHYSTPAYNFFDRPMLLSGLEGHHQEEKEYGGDAYLEHRRTLPLFPLHGEEHINGGGGSILKYGQSDGCDRYGRGPCASLKLCLN

SEQ ID NO: 157 - DNA, 909 bp, Brassica rapa
atggagccaccgcaacatcatcatcaagccgaccaagaaaacggcaacaacaacaagtccggctctggtagttatacgtgtcgtcaaacgagcacaagatggacaccaacaacggagcaaatcagaatcctaaaagatctttactacaacaacggagtccgatcaccaacggccgaccagattcagaagatctctgcaaggctgagactgtatgggaaaatcgagggtaaaaacgtcttttactggttccagaaccataaggctcgtgagcgacagaagaagagattcaacggcacagtcatgaccactccgacgtcttcatctcctaactcggttatgatggctagtgatcattattataaccatcatcctcttcttcaccatcctcagcaaggtgttaccatgcacagacctgcttcagtaaacgttaagcttgaccaagaaaatcatctgcttcaggaaaacagatcatatcctagcttcaataacggtacaggcactgaatgtggtgctcttaatgcgtcttctaatggctacatgaatagccatctctatggatctattgaacaagattgttcaatgagctacaacaacgtaggtggagagtggacaaacatggatcataatcatcattactcggctccagcttacaacttcttcgatagaccaaagcctatgtatgaactagaagttcatgaagaagaaggagactatggtggcgatgcttatctggaacatagacgtacccttcctctttttcctatgcacggtgaggatcacatcaacggcggtggtggtgtcatctggaagcacggacaatcggacggtcgtgatcgttatggtagaggcccttgtgcttctctaaagctatgcctgaactcccacgccgccggcgtcacacaggattaa

SEQ ID NO: 158 - protein, 302 aa, Brassica rapa
MEPPQHHHQADQENGNNNKSGSGSYTCRQTSTRWTPTTEQIRILKDLYYNNGVRSPTADQIQKISARLRLYGKIEGKNVFYWFQNHKARERQKKRFNGTVMTTPTSSSPNSVMMASDHYYNHHPLLHHPQQGVTMHRPASVNVKLDQENHLLQENRSYPSFNNGTGTECGALNASSNGYMNSHLYGSIEQDCSMSYNNVGGEWTNMDHNHHYSAPAYNFFDRPKPMYELEVHEEEGDYGGDAYLEHRRTLPLFPMHGEDHINGGGGVIWKHGQSDGRDRYGRGPCASLKLCLNSHAAGVTQD

SEQ ID NO: 159 - DNA, 936 bp, Brassica rapa
atggagcaaccgcaacatcatcaccatcaccatcaacaaaccgaccaagagagcggtaacaacaataagtccggctctggtggttacccatgtcgccaaacgagcacaagatggacaccaacgacggagcagatcagaatcctcaaagatctctactacaacaacggagttcggtcaccaacagccgaccagatccagaagatctctgcacggctgagacagtacggaaagatcgagggtaagaacgtcttttactggttccagaaccataaggctcgcgagcgacagaagaagagattcaacggcacaaccatgaccacaccaacaacttcatcccccaactcggttatgatggccaacgaccattatcatcataaccatcatcctcttcttcagcaccatcatcatggtgttaccatgcataggcctgcttcggtcaatattaagcttgaccaagaaaatcatctccttcatcagaacatatcttatcccaactttcagaacgggaatttaaatcatgcaagttcaggcgctgaatatggtgctcttaatgcttcttctaatggctacatgagtagccatctctatggatctatggaacaagattattcagtgagctacaacaacgtaagtggaggatggacaaacatggatcataatcatcattactcagctccagcttacaacttcttcgatagaccaaagcctctgtctggactggaagatcatgaagaagaagaatatggtggcgatgcttatctggaacatagtcgtacactttccctcttccctatgcacggtgaggataacatcaacggcggtggtggtgcctttttgaagtacggacaatcggacggtcgtgattattatggtagaggctcttgtgcttctctcaagttatgtttaaactcctacgcaggcgtctcaccggattaa

SEQ ID NO: 160 - protein, 311 aa, Brassica rapa
MEQPQHHHHHHQQTDQESGNNNKSGSGGYPCRQTSTRWTPTTEQIRILKDLYYNNGVRSPTADQIQKISARLRQYGKIEGKNVFYWFQNHKARERQKKRFNGTTMTTPTTSSPNSVMMANDHYHHNHHPLLQHHHHGVTMHRPASVNIKLDQENHLLHQNISYPNFQNGNLNHASSGAEYGALNASSNGYMSSHLYGSMEQDYSVSYNNVSGGWTNMDHNHHYSAPAYNFFDRPKPLSGLEDHEEEEYGGDAYLEHSRTLSLFPMHGEDNINGGGGAFLKYGQSDGRDYYGRGSCASLKLCLNSYAGVSPD

SEQ ID NO: 161 - DNA, 888 bp, Brachypodium distachyon
atggatggcgcggcggcgaacggggggcaggcggcgccgtgcagggcgagcgggacgcggtggacgccgacggcggagcaggtgcggatcctgcgggagctctactacggcctcgggatccggtcgcccaacgcggagcagatccagcggatcgcgggcaggctcaggcagtacggccggatcgaggggaagaacgtcttctactggttccagaaccacaaggcccgggaacggcacaagaagcgcctcaccaccatcgacgtctcccccaacaacaacgacagcaacaacaactgcagcaacaatgcctccatcctctccctctccccttcatcaggtgcggcggccggcttgtacggcgccggcagcaatggcggtggcggctcggctcatctgcagatggatgcgaatgcgagtgctacttgctgggacgtcggcagcaacgccgccatggccaacgacagaagcttcatgcagctgctgcaggagcaggactacatgggcgtgaggacgagcacggccgccatggcacccacgccgtggccggcatgcttcccggccttctccccgtatcagccaccgccggcgcgggagcccgagacgctgccgctcttccccaccggcggcggcagcagcggaggccaccaagaaatcgtcaacggcgtccatggcggaagctaccagctgctgcagtccaactcccagcagctgtgctgggggcagcaacatcatcatcatcatcagctgctgctgcaagagcagcagaactaccagtacagctcttacagcagcaacaaccagctgatgatgcccacccaagacgcagcagcagcagcctccttggagctcactctcagctcacattaccctgcagggtcatccatgtga

SEQ ID NO: 162 - protein, 295 aa, Brachypodium distachyon
MDGAAANGGQAAPCRASGTRWTPTAEQVRILRELYYGLGIRSPNAEQIQRIAGRLRQYGRIEGKNVFYWFQNHKARERHKKRLTTIDVSPNNNDSNNNCSNNASILSLSPSSGAAAGLYGAGSNGGGGSAHLQMDANASATCWDVGSNAAMANDRSFMQLLQEQDYMGVRTSTAAMAPTPWPACFPAFSPYQPPPAREPETLPLFPTGGGSSGGHQEIVNGVHGGSYQLLQSNSQQLCWGQQHHHHHQLLLQEQQNYQYSSYSSNNQLMMPTQDAAAAASLELTLSSHYPAGSSM

SEQ ID NO: 163 - protein, 320 aa, Zea mays
MAANVGAGRSAGGGGAGTGTGTAAGSGGVSTAVCRPSGSRWTPTPEQIRILKELYYGCGIRSPNSEQIQRITAMLRQHGKIEGKNVFYWFQNHKARERQKRRLTNLDVNVPVAADDSAHRLGVLSLSPSSGCSGAAPPSPTLGFYAGGNGSAVMLDTSSDWGSAAAMATEACFMQDYMGVMGGASPWACSSSSSEDPMAALALAPKVTRAPETLPLFPTGGGGDDRQPPRPRQSVPAGEAIRGGSSSSSYLPFWGAAPTPTGSATSVAIQQQHQLMQMQEQYSFYSNAQLLPGTGSQDAAATSLELSLSSWCSPYPAGTM

SEQ ID NO: 164 - DNA, 870 bp, Oryza sativa
atggatcacatgcagcagcagcagcggcagcaggtgggtggagggggaggagaggaggtggcggggaggggtggtgtgccggtgtgccggccgagcgggacgaggtggacgccgacgacggagcagatcaagatcctgcgggagctgtactacagctgcggcatcaggtcgcccaactcggagcagatccagcggatcgccgccatgctgcgccagtacggccgcatcgagggcaagaacgtcttctactggttccagaaccacaaggcccgcgagcgccagaagaagcgcctcaccacgctcgacgtcaccaccaccaccgccgccgccgccgacgccgacgccagccacctcgccgtcctctccctctcgcctacagcagctggcgcgacggctccctctttcccgggcttctacgtcggcaatggcggcgccgtgcagacggatcaggccaacgtcgtcaactgggactgcaccgccatggcagccgagaaaaccttcctgcaggactacatgggcgtgagcggcgtcggttgcgccgccggggcggccccgacgccgtgggcgatgacgacgacgactcgcgagcccgagacgcttccactcttcccagtcgtcttcgtcggcggcgacggcgcgcatcgtcacgccgtccacggcggtttcccgtccaacttccagcgctggggttctgctgctgctacctccaacaccattacggtccagcagcatttgcagcagcacaacttttacagcagcagcagcagccagctgcacagccaggatgggccggcagcaggcacatccctggagctcactctcagctcctactactgctcatgctcaccttaccctgcagggtccatgtga

SEQ ID NO: 165 - protein, 289 aa, Oryza sativa
MDHMQQQQRQQVGGGGGEEVAGRGGVPVCRPSGTRWTPTTEQIKILRELYYSCGIRSPNSEQIQRIAAMLRQYGRIEGKNVFYWFQNHKARERQKKRLTTLDVTTTTAAAADADASHLAVLSLSPTAAGATAPSFPGFYVGNGGAVQTDQANVVNWDCTAMAAEKTFLQDYMGVSGVGCAAGAAPTPWAMTTTTREPETLPLFPVVFVGGDGAHRHAVHGGFPSNFQRWGSAAATSNTITVQQHLQQHNFYSSSSSQLHSQDGPAAGTSLELTLSSYYCSCSPYPAGSM

SEQ ID NO: 166 - DNA, 909 bp, Medicago truncatula
atggaacagcctcaacaacaacaacaaccacagacacaacaacattcaccaaacaatgggattatgggaagcagacaaagtagtacaaggtggacaccaacaacagatcagataagaatattgaaggatctttactacaacaatggaattagatcaccaagtgcagaacagattcagagaatctctgctaggttaagacagtatggtaagattgaaggcaaaaatgtcttttattggtttcaaaatcacaaagctagagaaagacagaagaaaaggttcacttctgatgttaatgttgttcccatcattcaaagagcaccaaacaacaatactattatttctgctgctaattggaaacctgatcatcatgaacaacaacaaaacattaatgttcataccaaccattctacttacaacatttcatctgctgggctttcttctgcttcatgttcttctgctgagatggttactgtaggccagattggtaactatggatatggatctgtccccatggaaaagagttttagggagtgtacaatttcagctggatgtagcagcagtcaagttggaagtaccataaaccctcacataggatggattggtcatcatgtcgatccatattcttcagcttatgccaacttatttgaaaaaataagaccaaatgaagaaatcatggaagaatatgatcaaggacaagaaaatggttcacctgaaattgaaaccctccctttattccctatgcatggtgaagacattcatggtggctattgtaacctcaaatcaaattcatctaactatggtggttggtatcaagctgaagatgctggattcatgtatggttctcgtactacttccttggaacttagcctcaactcttacggctgtaggtcaccagattatgctaattaa

SEQ ID NO: 167 - protein, 302 aa, Medicago truncatula
MEQPQQQQQPQTQQHSPNNGIMGSRQSSTRWTPTTDQIRILKDLYYNNGIRSPSAEQIQRISARLRQYGKIEGKNVFYWFQNHKARERQKKRFTSDVNVVPIIQRAPNNNTIISAANWKPDHHEQQQNINVHTNHSTYNISSAGLSSASCSSAEMVTVGQIGNYGYGSVPMEKSFRECTISAGCSSSQVGSTINPHIGWIGHHVDPYSSAYANLFEKIRPNEEIMEEYDQGQENGSPEIETLPLFPMHGEDIHGGYCNLKSNSSNYGGWYQAEDAGFMYGSRTTSLELSLNSYGCRSPDYAN

SEQ ID NO: 168 - DNA, 864 bp, Phseolus vulgaris
atggaacctcaacaaccacaagggagccaaccaaacgaggatggtggcagtggaaaagggggctttcttagcaggcaaagtagtacacggtggactcccacaaccgaccagataagaatattgaaggacctttactacaacaatggaattagatccccaagtgcagagcagattcagaggatctctgctaggctgaggcagtacggtaagattgaaggcaagaatgtcttttattggttccagaaccacaaagctcgagaaaggcagaagaaaaggttcacttctgataatgtgcccatgcaaagacctgccccaactaatgctgctcctccttggaaacctgatcaagatcctattcataccaagtattccaacatttcttctacagggatctcttctgcatcatcttcttctgttgagatgattactgtcgggcagatggggaattatggctatggttctgtacccatggagaaaagttttagggactgttcaatatctgctgggggtagcagtggccatgttggaataaaccacaacttgggatgggttggtgttgatccttattcctcagcctacgccaacttctttgataaaataaggccaaatgaagaaaccctggaagaagaagaagaagaagaagaggaagaagaggaggaggatgggggtgctgagattgaaactctccctttattccctatgcacggcgaggacattcatggctactgcaacctgaagtctaattcttacaactatgatggaaacgggtggtatcattctgaagacgggttcaagaatggttctcgtgcttctttggagctcagtctgaactcctacactcgcagatctccagatttcgcttaa

SEQ ID NO: 169 - protein, 287 aa, Phseolus vulgaris
MEPQQPQGSQPNEDGGSGKGGFLSRQSSTRWTPTTDQIRILKDLYYNNGIRSPSAEQIQRISARLRQYGKIEGKNVFYWFQNHKARERQKKRFTSDNVPMQRPAPTNAAPPWKPDQDPIHTKYSNISSTGISSASSSSVEMITVGQMGNYGYGSVPMEKSFRDCSISAGGSSGHVGINHNLGWVGVDPYSSAYANFFDKIRPNEETLEEEEEEEEEEEEEDGGAEIETLPLFPMHGEDIHGYCNLKSNSYNYDGNGWYHSEDGFKNGSRASLELSLNSYTRRSPDFA

SEQ ID NO: 170 - DNA, 984 bp, Sorghum bicolor
atggcggccaatgtgggcgcgggcaggagtgccgtcggcggcggcggtggtggtggagcgggtggcactggcactgctgctgccagcggcagcgtggcgacgacagcggtgtgccgccccatcggctcgcggtggacgccgacgcccgagcagatcaggatactcaaggagttctactacggctgcggcatccggtcgcccaactcggagcagatccagcgcatcaccgccatgctgcgccagcacggcaagatcgagggcaagaacgtcttctactggttccagaaccacaaggcccgcgaacgccagaagcgccgcctcaccaacctcgacgtcaacgtgcccaccgccgcagccgccggcgcggccgacgccagcacccacctaggcgtcctctcgctgtcgtcgccttcaggcgcggcgcctccctcgcccaccctcggcttctacgccggcaatggcggcgccggctcgaccgtgctgctggacacgagttccgactgcgctgccatggccactgagacatgcttcctgcaggactacatgggcgtgatgggcacgggcagcgcggccgccgcgtcgccgtgggcatgcttctcgtcgtcgaacactatggcggcggccgcggcacgggcaccgacggtgacgcgggcgcccgagacgctccctctcttcccgacgggcggcgacgacagccagccccggcggccgcggcacggagtcccagttccagttgcagcaggcgaggccatccgcggcggcagcagcagcagcaggtacctgccgttctggggtgccgcgcccacaactgccagtgccacttccattgggatccagcagcaacaccagctgctgcagctgcaagagcagtacagcttttacagcaacgccatgcccggcaccggcagccaagatgcatcagcagcatccctggagctcagcctcagctcctggtgctccccttaccctgcagggaccatgtga

SEQ ID NO: 171 - protein, 327 aa, Sorghum bicolor
MAANVGAGRSAVGGGGGGGAGGTGTAAASGSVATTAVCRPIGSRWTPTPEQIRILKEFYYGCGIRSPNSEQIQRITAMLRQHGKIEGKNVFYWFQNHKARERQKRRLTNLDVNVPTAAAAGAADASTHLGVLSLSSPSGAAPPSPTLGFYAGNGGAGSTVLLDTSSDCAAMATETCFLQDYMGVMGTGSAAAASPWACFSSSNTMAAAAARAPTVTRAPETLPLFPTGGDDSQPRRPRHGVPVPVAAGEAIRGGSSSSRYLPFWGAAPTTASATSIGIQQQHQLLQLQEQYSFYSNAMPGTGSQDASAASLELSLSSWCSPYPAGTM

SEQ ID NO: 172 - DNA, 978 bp, Zea mays
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccttcaggcgcggcgcctccctcgcccaccctcggcttctacgccgccggcaatggcggcggatcggctgggctgctggacacgagttccgactggggcagcagcggcgctgccatggccaccgagacatgcttcctgcaggactacatgggcgtgacggacacgggcagctcgtcgcagtggccatgcttctcgtcgtcggacacgataatggcggcggcggcggccgcggcgcgggtggcgacgacgcgggcgcccgagacactccctctcttcccgacctgcggcgacgacgacgacgacgacagccagcccccgccgcggccgcggcacgcagtcccagtcccggcaggcgagaccatccgcggcggcggcggcagcagcagcagctacttgccgttctggggtgccggtgccgcgtccacaactgccggcgccacttcttccgttgcgatccagcagcaacaccagctgcaggagcagtacagcttttacagcaacagcacccagctggccggcaccggcagccaagacgtatcggcttcagcggccgccctggagctgagcctcagctcatggtgctccccttaccctgctgcagggagcatgtga

SEQ ID NO: 173 - protein, 325 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPSGAAPPSPTLGFYAAGNGGGSAGLLDTSSDWGSSGAAMATETCFLQDYMGVTDTGSSSQWPCFSSSDTIMAAAAAAARVATTRAPETLPLFPTCGDDDDDDSQPPPRPRHAVPVPAGETIRGGGGSSSSYLPFWGAGAASTTAGATSSVAIQQQHQLQEQYSFYSNSTQLAGTGSQDVSASAAALELSLSSWCSPYPAAGSM

SEQ ID NO: 174 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 175 - protein, 238 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGLYAAGNGGGSAVLLDTSSDWGSSGAAMATETCFLQVGAVVRSFLGHCAQFHVRTYELIAASFHPPVYITVRYGGARPQDYMGVTDTGSSSQWPRFSSSDTIMA

SEQ ID NO: 176 - protein, 237 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQVGAVVRSFLGHCAQFHVRTYELIAASFHPPVYITVRYGGARPQDYMGVTDTGSSSQWPRFASSDTIM

SEQ ID NO: 177 - DNA, 1176 bp, Escherichia coli
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 178 - DNA, 690 bp, Artificial sequence
  synthetic
atggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctga

SEQ ID NO: 179 - DNA, 2097 bp, Setaria italica
atggctactgtgaacaactggctggctttctccctctccccgcaggatctgccgccttcccagacggattccaccctcatctccgccaccgccaccgacgaggtctccggcgacgtctgcttcaacatcccccaagattggagcatgaggggatcggaactctcagcgctagtcgccgagccgaagctggaggacttcctcggcggcatcaacttctcggagcagcaccacaaggccaacctcaacgtgatccccagcagtagtaacgcttgctacgccagctcaggcgctagcacaggctaccaccagctgtaccaccaccaaagctccgcgctccatttcgctgactccgtcatggtggcctcctccgccggcgtccatgacggcggtgccagcatgctcagtgcagcggccaccgtcaatggcggcgctggcgctgccggcgccaacggcggcagcatcgggctgtccatgatcaagaactggctgcggagtcaaccagctccgccgctgccgcaaccgagagtagtggcggccgccgagggcgcgcaggcggcgcaggggctttctttgtccatgaacatggcaggggcgcaaggcgctggcatgccgcttctcgccggagagcgaggccgggcgcccgagagcgtgtccacatcagcacagggtggagcagtcgccgctcggaaggaggatagcggtagcagcggcggtgccggcgccctactagccgtgagcacagacacgggtggcagcggcacggttgctgagacggcggcgaggaagacggtagacacgttcggccagcgcacgtcgatttaccgcggggtgaccaggcatagatggacagggaggtacgaggctcatctgtgggacaacagttgcagaagggaaggacaaactcgcaagggtcgtcaagtctatctaggtggttatgacaaagaggagaaagctgctagggcttatgatttggctgctcttaagtattggggtccgacgacgaccacaaattttccggtgagtaactacgaaaaggagctggaggaaatgaagcacatgacaaggcaggagtttgtagcgtctctgagaaggaagagcagtggcttctccagaggtgcatccatttacagaggagtaactaggcatcaccaacatgggagatggcaagcacggattggacgagttgcagggaacaaggatctctacttgggcaccttcagcacgcaggaggaggcagcagaggcgtacgacatcgcggcgatcaagttccgcgggctcaacgccgtcaccaacttcgacatgagccgctacgacgtgaagagcatcctggacagcagcgcgctccccatcggcagcgccgccaagcgcctcaaggaggccgaggccgccgcgtccgcgcagcacgctggcgtggtgagctacgacgtaggccgcatcgcctcgcacctcggcgacggcggcgccctggcagcctacggcacgcactaccacgcagccgcagccgcctggccgaccatcgcgttccagccgagcgccgccgccgccggcctgtaccacccgtacgcgcagccgatgcgtgggtggtgcaagcaggagcaggatcacgcggtgatcgccgccgcgcacagcctgcacgagctgaaccacctgaacctgggcgccggcggcggtgcgcacgacttcttctcggcggggcaggccgcgatgcacggcctcggcagcatcgacaatgcctcactcgagcatagcaccggctccaactccgtcgtgtacaatggggttggggacagcaacggtggcgccgtcggcggcggctacatgatgccgatgagcgctgccacggcgaccacgacggcaatggtgagtcacgagcaggtgcatgcgcggtcacatcaaggggagcacgacgaagccaccaagcaggcggcgcagatggggtacgagagctacctggtgaacgcggaggcggcctacggtggcgggaggatgccttcgtggacgccggccagcgccccggcagcggcaagcagcaacgacaacatggccggcgttggccacggcggcgcgcagctcttcagtgtctggaatgatacataa

SEQ ID NO: 180 - DNA, 2064 bp, Brachypodium distachyon
atggctaccgtgaacaactggctgggcttctccctctccccgcaggagctgccgtcctccgccgctgccgccgccggcgacgtctccggcgccgacgtctgcttcaacatcccgcaagattggggcatgaggggatcggagctgtcggcgctggtggcggagccgaagctggaggacttcctcggcggcatctcttcctactctgaccatcacaaggcagccagaagcaacaacatgaacatcaatggcgccgccgcctgctacgccagctccggcagcagcggctaccagctttaccacgaccaccccaactccctccagttcgccgactctgtcatggtcgcttcctccgctggcggcgtccacaatgaacatggcatcatggcaagcaccactgccaatggtgcaggcaccaatggcggcatcgggctgtccatgatcaagagctggctgcggagccagccggcgccggcgcagcaggagcagcagagggcggaggggctgtcgctgtccatgaacatgccgctcctgcaggcggcggcggcggagactagcttgtccacgtcggcagcgcagcagggatctcggaaggagaataatggcagcagtagtgctggttctggagccgtggtgtcagccggtactaccagcgccggcgcggtggtggtggagtcgccggcagcggggaggaagaccgccgacacgttcgggcagcggacttcgatctaccgcggcgtcaccaggcatagatggacagggaggtatgaggctcacctgtgggacaacagctgcagaagagaaggacaaactcgcaagggtcgtcaagtttatctaggtggttatgacaaagaggagaaagctgcaagagcttatgatttggctgctctcaagtattggggccccaccacgacgacaaattttccagtagataactacgagaaggagctggaggagatgaagcacatgacaaggcaggagtttgtggcgtctctccgaaggaagagcagtggtttttcaagaggtgcatccatttatcgtggagtaactaggcaccaccaacatgggagatggcaagcaaggatagggagagttgcagggaacaaggatctctacttgggcactttcagcacgcaggaggaggcggcggaggcgtacgacatcgcggcgatcaagttccgggggctgaacgccgtcactaacttcgacatgtcccgctacgacgtcaagagcatcctcgacagcaccgccgcgctgcctgtcggcggcgccaagcgcctcagggacgcggctgccgccgatcagcactaccagcagcgcgccgggggcgtcgtcagctacgccgcgccgcagctcggtggtgtcaacgagaccgcccttgcctatggcgcgccctactaccaccaccaaacctccgccgcagcgtggccgaccatcgcgttccaggcggcgccgcaagcgtcgtcggggcacgggcacatgctgtaccacccgtacgggcagccattgatgcgcgggtggtgcaagcaagagcaggaacaagggcaagggcagcaggagccggaccacgcggtgatcgcggccgcgcacagcctgcaggacctccaccacctcaacctcggcgccggtgcgcacgacttcttctcccagcatgcccacgccatgcaccagcagcagcagcagcacggcggcctcggcagcgtcgacaacaacggcgcggcgtcgctggagcacagcactggctccaactccgtcgtctacaacggggccgctgccgccggggacaccaacaacagctacatgctgccgcccatgagcgctgcagcagcagctggcttcggcctccgcgatcagcaggacgaaggcgggaagatggcgtacgagaacttcctcctcggcgccgccaccgacggctactgtggccctggaaggatggcggccacctggacaccggtgtcggtttcggcggcccagccagtggcggcaacgagcagcggcagcgacatggccggtgccgtctgccacggcggcgcgcagctcttcagcgtctggaacgacgacagttag

SEQ ID NO: 181 - DNA, 1740 bp, Brassica napus
atggctaataattggcttggtttctctctttcaccatacgagcagaatcatcatcggaaggacgtttactcatccaccactacgaccgtggtcgacgtggcgggcgagtactgctacgacccgacggcggcctcggacgagtccagcgcgatccagacgtccttccccagcccattcggcgtggtcgtggacgctttcaccagggacaacaactcccacagcagggactgggacatcaacggctgcgcctgcaacaacatccataacgacgagcaggacggcccgaagctcgagaacttcctgggccgcaccacgaccatctacaatactaacgagaacgtgggcgacggcagcggcagcgggtgctacggtggtggcgacggcagcggcggctccctcggcctgagcatgatcaagacgtggctgcgcaaccagcccgtggacaacgtcgacaaccaggagaacggcaacgcggccaagggcctctccctgagcatgaactccagcacctcctgcgacaacaacaacgattccaacaacaacgtggtcgcccagggcaagacgatcgacgactccgtggaggcgaccccgaagaagacgatcgagtccttcggccagcgcacttctatctacaggggcgtcacccgccacaggtggaccggccgctacgaggcgcacctctgggataacagctgcaagagggagggccagacccgcaagggcaggcaggtgtacctgggcggctacgacaaggaggagaaggcggccagggcttacgacctcgcggccctgaagtactggggcacgaccacgaccacgaacttcccgatgtccgagtacgagaaggaggtggaggagatgaagcacatgacccgccaggagtatgtggctagcctcaggaggaagtccagcggcttctccaggggcgcgagcatctaccgcggcgtgacgaggcaccaccagcacggcaggtggcaggctcgcatcggcagggtcgcgggcaacaaggacctctacctgggcaccttcggcacgcaggaggaggctgccgaggcttacgacatcgctgcgatcaagttccgcggcctcaccgcggtgacgaacttcgacatgaacaggtacaacgtcaaggccatcctcgagagccccagcctgccaatcggctccgctgccaagaggctgaaggaggccaacaggccggtgccctccatgatgatgatcagcaacaacgtctccgagtcggagaactcggcttcgggctggcagaacgcggccgtgcagcaccaccagggcgtcgacctctccctcctccaccagcaccaggagaggtacaacggctactactacaacggcggcaacctgtccagcgagtcggctagggcctgcttcaagcaggaggacgaccagcaccacttcctctccaatacacagagcctgatgacgaacatcgaccaccagtccagcgtgtccgacgacagcgtgacggtctgcggcaacgtggtgggctacggcggctaccagggcttcgcggccccagtcaactgcgacgcctacgccgcgagcgagttcgactacaacgcccgcaaccactactacttcgcgcagcagcagcagacccagcagtccccgggcggcgacttcccggctgcgatgacgaacaacgtgggcagcaacatgtactaccacggcgagggcggcggcgaggttgctccgactttcactgtttggaacgataattaa

SEQ ID NO: 182 - DNA, 2097 bp, Setaria italica
atggctactgtgaacaactggctggctttctccctctccccgcaggatctgccgccttcccagacggattccaccctcatctccgccaccgccaccgacgaggtctccggcgacgtctgcttcaacatcccccaagattggagcatgaggggatcggaactctcagcgctagtcgccgagccgaagctggaggacttcctcggcggcatcaacttctcggagcagcaccacaaggccaacctcaacgtgatccccagcagtagtaacgcttgctacgcgagctcaggcgctagcacaggctaccaccagctgtaccaccaccaaagctccgcgctccatttcgctgactccgtcatggtggcctcctccgccggcgtccatgacggcggtgccagcatgctcagtgcagcggccaccgtcaatggcggcgctggcgctgccagtgccaacggcggcagcatcgggctgtccatgatcaagaactggctgcggagtcaaccagctccgccgctgccgcaaccgagagtagtggcggccgccgagggcgcgcaggcggcgcaggggctttctttgtccatgaacatggcaggggcgcaaggcgctggcatgccgcttctcgccggagagcgaggccgggcgcccgagagcgtgtccacatcagcacagggtggagcagtcgccgctcggaaggaggatagcggtagcagcggcggtgccggcgccctactagccgtgagcacagacacgggtggcagcggcacggttgctgagacggcggcgaggaagacggtagacacgttcggccagcgcacgtcgatttaccgcggggtgaccaggcatagatggacagggaggtacgaggctcatctgtgggacaacagttgcagaagggaaggacaaactcgcaagggtcgtcaagtctatctaggtggttatgacaaagaggagaaagctgctagggcttatgatttggctgctcttaagtattggggtcccacgacgaccacaaattttccggtgagtaactacgaaaaggagctggaggaaatgaagcacatgacaaggcaggagtttgtagcgtctctgagaaggaagagcagtggcttctccagaggtgcatccatttacagaggagtaactaggcatcaccaacatgggagatggcaagcacggattggacgagttgcagggaacaaggatctctacttgggcaccttcagcacgcaggaggaggcagcagaggcgtacgacatcgcggcgatcaagttccgcgggctcaacgccgtcaccaacttcgacatgagccgctacgacgtgaagagcatcctggacagcagcgcgctccccatcggcagcgccgccaagcgcctcaaggaggccgaggccgccgcgtccgcgcagcacgctggcgtggtgagctacgacgtaggccgcatcgcctcgcacctcggcgacggcggcgccctggcagcctacggcacgcactaccacgcagccgcagccgcctggccgaccatcgcgttccagccgagcgccgccgccgccggcctgtaccacccgtacgcgcagccgatgcgtgggtggtgcaagcaggagcaggatcacgcggtgatcgccgccgcgcacagcctgcacgagctcaaccacctgaacctgggcgccggcggcggtgcgcacgacttcttctcggcggggcaggccgcgatgcacggcctcggcagcatcgacaatgcctcactcgagcatagcaccggctccaactccgtcgtgtacaatggggttggggacagcaacggtggcgccgtcggcggcggctacatgatgccgatgagcgctgccacggcgaccacgacggcaatggtgagtcacgagcaggtgcatgcgcggtcacatcaaggggagcacgacgaagccaccaagcaggcggcgcagatggggtacgagagctacctggtgaacgcggaggcggcctacggtggcgggaggatgccttcgtggacgccggcctcggccccggcagcggcaagcagcaacgacaacatggccggcgttggccatggcggcgcgcagctcttcagtgtctggaatgatacataa

SEQ ID NO: 183 - DNA, 2082 bp, Panicum virgatum
atggctactgtgaacaactggctggctttctccctctcccagcaggatctgccaccatcccagacggactccaccctcatctccgccgccgccaccgacgaggtctccggcgacgtctgcttcaacatcccccaagattggagcatgaggggatcggagctttcagcgctcgtcgccgagccgaagctggaggacttcctcggcggcatcaacttctcggagcagcatcacaaggccaacctcaacgtgatccccagcagtagcagcacttgctacgcgagctcaggtgctagcaccggctaccaccagctgtaccaccaccccagctccgcgctccatttcgctgactccgtcatggtggcctcttccgccggcgtccatgatggcggtgcaatgctcagcgcggccaccgcccatggtggagctggcgctgccggtgccaatggcggcagcatcggtctgtccatgatcaagaattggctgcggagccaaccagctccgccgccgcagcccagagtggcggtgcccgagggagcgcaggcggtgcagggcctctctttgtccatgaacatggcggggacgcaaggcgctggcatgccgtttctcgccggagatcgaggccgggcgcccgagagcgtatccacgtcggcgcagggtggagccgtcgccgctcggaaggaggacagcggtggcagcggcggtgccggcgccctagtagtagcagtgagcacggacacgggtggcagtggcagcggcggcgcgtctgctgagacggcggcgaggaagacggtggacacgttcggccagcgcacgtcgatttaccgcggcgtgaccaggcatagatggacggggaggtacgaggctcatctgtgggacaacagttgcagaagggaaggacaaactcgcaagggtcgtcaaggtggttatgacaaagaggagaaagctgctagggcttatgatttggctgctcttaagtattggggtcccacgacgacaacaaactttccagtgagtaactacgaaaaggagttggaggaaatgaagcacatgacaaggcaggagtttgttgcgtctctgagaaggaagagcagtggcttctccagaggtgcatccatttacagaggagtaactaggcatcatcaacatgggagatggcaagcacggattggacgagttgcagggaacaaggatctctatttgggcaccttcagcacgcaggaagaggcagcggaggcatacgacatcgcggcgatcaagttccgcggccttaacgccgtcaccaacttcgacatgagccgctacgacgtgaagagcatcctggacagcagcgcgctccccatcggcagcgccgccaagcgcctcaaggaggccgaggccgccgcgtccgcgcagcaccatgctggcgtggtgagctacgacgtcggccgcatcgcctcgcagctcggcgacggcggcgccctggcggcctacggcgcgcactaccacgccgccgcggccgctgcctggccgaccatcgcgtttcagccgggcgccgccgccggtggcctgtaccacccgtacgcgcagccgctgcgcgggtggtgcaagcaggagcaggaccacgcggtgatcgcggccgcgcacagcctgcaggagctcaaccacctgaacctgggagccggcgcgcacgacttcttctcggcggggcaggcggcgatgcacggcctcggcagcatcgacaactcctcgctcgagcatagcaccggctccaactccgtcgtgtacaatggggtaggagacagcaacggcggtggcggctacatgatgccgatgaatgctgccgcggcgaccaccacggcaatggtgaaccatgagcaggtgcatgcacgggcacatggggaccacgacgaagccagcaaacaggtgatggggtatgagagctacctggtcaacgcagaggcggcctacggtggcgggaggatgccttcctggacgacggcctccgcttcaccggtagcggcagcggctgcaagcagcaacgacaacatggccggcgttggccatggcggtgcgcagctcttcagtgtctggaatgacacttaa

SEQ ID NO: 184 - DNA, 2001 bp, Panicum virgatum
atggctactgtgaacaactggctggctttctccctctccccgcaggatctgccgccttcccagacggactccaccctcatctccgccgccgccaccgacgaggtctccggcgacgtctgcttcaacatcccccaagattggggcatgaggggatcggagctttcagcgctcgtctccgagcccaagctggaggacttcctcggcggcatcaacttctcggagcagcatcacaaggccaacctcaacgtgatccccagcagtagcagcacttgctacgcgagctcaggtgctagctccgccggctaccaccagctgtaccaccaccccagctccgcgctccatttcgctgactccgtcatggtggcctcatccgccggcgtccatgacggcggtgccatgatcagtgcggccaccgccagtggtggagctggcgctgccggcgccaatggcggcagcatcgggctgtccatgatcaagaactggctgcggagccaaccagctccggcgccgccgcagccgagagtggcggtggccgagggcgcgcaggcggcgcagggtctctctttgtccatgaacatggcggggacgcaaggcgctggcatgccgtttctcgccggtgagcgaggccgggcgcccgagagcgcatccacgtcggcacagggtggagccgtcgccgctcggaaggaggacagcggtggcagcggcggtgccggcgccctagtaacagtgagcacggacacgggtggcagcggcggcgcgtctgctgagacggcggcgaggaagacggtggacacgttcggccagcgcacgtcgatttaccgtggcgtgaccaggcatagatggacggggaggtacgaggctcacctgtgggacaacagttgcagaagggaaggacaaactcgcaagggtcgtcaagtctatctaggtggttatgacaaagaggagaaagctgctagggcttatgatttggctgctcttaagtattggggtcccacgacgacaacaaattttccggtgagtaactacgaaaaggagttggaggaaatgaagcacatgacaaggcaggagtttgttgcttctctgagaagtggcattttcaaatctgatcccatgtctggcacgcaggaggaggcagcggaggcgtacgacatcgcggcgatcaagttccgcggcctcaacgccgtcaccaacttcgacatgagccgctacgacgtgaagagcatcctggacagcagcgcgctccccatcggcagcgccgccaagcgcctcaaggaggccgaggccgccgcgtccgcgcagcaccatgccggcgtggtgagctacgacgtcggccgcatcgcctcgcagcttggcgacggcggcgccctggcggcctacggcgcgcactaccacgccgccgccgccgcctggccgaccatcgcgttccagccgggcgccgccgccgccggcctgtaccacccgtacgcgcagcctctgcgcgggtggtgcaagcaggagcaggactacgccgtgatcgcggccgcgcacagcctgcaggagctcaaccacctgaacctgggcgccggcgcgcacgacttcttctcggcggggcaggcggcgatgcacggcctcggcagcatcgacaactcctcgctcgagcatagcaccggctccaactccgtcgtgtacaacggggttggggacagcaacggtggcgccgtcggcggcggctacatgatgccgacgagcgccgccgccgcggcgaccaccacggcgatggtgagccacgagcaggtgcacgcacgggcacagggggaccacgacgaagccagcaaacgggcggcgcatatggggtatgagagctacctagtcaacgcagaggcggcctacggtggcgggaggatgccttcctggacgacggcctcggctccaccggtggcggcagcggcaagcagcaacgacaacatggccggcgttggccatggcggcgcgcagctcttcagtgtctggaatgacacttaa

SEQ ID NO: 185 - DNA, 1680 bp, Oryza sativa
atggcctccatcaccaactggctcggcttctcctcctcctccttctccggcgccggcgccgaccccgtcctgccccacccgccgctgcaagagtgggggagcgcttatgagggcggcggcacggtggcggccgccggcggggaggagacggcggcgccgaagctggaggacttcctcggcatgcaggtgcagcaggagacggccgccgcggcggcggggcacggccgtggaggcagctcgtcggtcgttgggctgtccatgatcaagaactggctacgcagccagccgccgcccgcggtggttgggggagaagacgctatgatggcgctcgcggtgtcgacgtcggcgtcgccgccggtggacgcgacggtgccggcctgcatttcgccggatgggatggggtcgaaggcggccgacggcggcggcgcggccgaggcggcggcggcggcggcggcgcagaggatgaaggcggccatggacacgttcgggcagcggacgtccatctaccggggtgtcaccaagcacaggtggacaggaaggtatgaagcccatctttgggataacagctgcagaagagaaggtcagactcgcaaaggcagacaagtcaatgcaggaggatatgataaggaagaaaaagctgctagggcttatgatttggctgcccttaaatactggggcactacaacgacgacgaattttccggtaagcaactacgaaaaagagttggatgaaatgaagcacatgaataggcaggaatttgttgcatcccttagaagaaaaagcagtggattttcacgtggtgcttccatatatcgtggtgttacaagacaccatcagcatggaaggtggcaagcaaggataggacgggtggcaggaaacaaggatctgtatttgggcacatttggcacccaagaggaagctgcagaggcatatgatatcgctgcaatcaaattccgtggtctcaatgctgtgacaaactttgacatgagccggtacgatgtcaagagcatcattgaaagcagcaatctcccaattggtactggaaccacccggcgattgaaggactcctctgatcacactgataatgtcatggacatcaatgtcaataccgaacccaataatgtggtatcatcccacttcaccaatggggttggcaactatggttcgcagcattatggttacaatggatggtcgccaattagcatgcagccgatcccctcgcagtacgccaacggccagcccagggcatggttgaaacaagagcaggacagctctgtggttacagcggcgcagaacctgcacaatctacatcattttagttccttgggctacacccacaacttcttccagcaatctgatgttccagacgtcacaggtttcgttgatgcgccttcgaggtccagtgactcatactccttcaggtacaatggaacaaatggctttcatggtctcccgggtggaatcagctatgctatgccggttgcgacagcggtggaccaaggtcagggcatccatggctatggagaagatggtgtggcaggcattgacaccacacatgacctgtatggcagccgtaatgtgtactacctttccgagggttcgcttcttgccgatgtcgaaaaagaaggcgactatggccaatctgtggggggcaacagctgggttttgccgacaccgtag

SEQ ID NO: 186 - DNA, 2103 bp, Oryza sativa
atggcttctgcaaacaactggctgggcttctcgctctccggccaagagaatccgcagcctcaccaggatagctcgcctccggcagccatcgacgtctccggcgccggcgacttctatggcctgccgacgtcgcagccgacggcggccgacgcgcacctcggcgtggcggggcatcatcacaacgcctcgtatggcatcatggaggccttcaataggggagctcaagaggcacaagattggaacatgagggggctggactacaacggcggcgcctcggagctgtcgatgctcgtcggctccagcggcggcaagagggcggcggcggtggaggagaccgagccgaagctggaggacttcctcggcggcaactcgttcgtctccgagcaagatcatcacgcggcggggggcttcctcttctccggcgtcccgatggccagcagcaccaacagcaacagcgggagcaacactatggagctctccatgatcaagacctggctccggaacaacggccaggtgcccgccggccaccagccgcagcagcagcagccggcggccgcggccgccgccgcgcagcagcaggcgcacgaggcggcggagatgagcaccgacgcgagcgcgagcagcttcgggtgctcctccgacgcgatggggaggagtaacaacggcggcgcggtctcggcggcggccggcgggacgagctcgcagagcctggcgctctcgatgagcacgggctcgcactcgcacctgcctatcgtcgtcgccggcggcgggaacgccagcggcggagcggccgagagcacatcgtcggagaacaagcgggccagcggcgccatggattcgccgggcggtggcgcgatagaggccgtgccgaggaagtccatcgacacgttcgggcaaaggacctcgatatatcgaggtgtaacaaggcatagatggacagggcgatatgaggctcatctctgggataatagctgtagaagagaagggcagagtcgcaagggtaggcaaggtggctatgacaaggaggataaagcagcgagagcttatgatttggcagctctgaagtattggggcacaacaacaacaacaaatttcccaataagtaactatgaaaaagagctagatgaaatgaaacatatgaccaggcaggagtatattgcatacctaagaaggaatagcagtggattttctcgtggtgcatcgaaatatcgtggtgtaaccaggcaccatcagcatgggagatggcaagcaaggatagggagggttgcaggaaacaaggacctctacttaggcaccttcagcaccgaggaggaggcggcggaggcgtacgacatcgcggcgatcaagttccgggggctcaacgccgtcaccaactttgacatgagccgctacgacgtcaagagcatcctggagagcagcacgctgccggtgggcggcgcggcgaggcggctgaaggaggcggcggaccacgcggaggcggccggcgccaccatctggcgcgccgccgacatggacggcgccggcgtcatctccggcctggccgacgtcgggatgggcgcctacgccgcctcgtaccaccaccaccaccaccacggctggccgaccatcgcgttccagcagccgccgccgctcgccgtgcactacccgtacggccaggcgccggcggcgccgtcgcgcgggtggtgcaagcccgagcaggacgccgccgtcgctgccgccgcgcacagcctccaggacctccagcagctgcacctcggcagcgccgccgcccacaacttcttccaggcgtcgtcgagctcgacggtctacaacggcggcggcggcgggtaccagggcctcggtggcaacgccttcttgatgccggcgagcaccgtcgtggccgaccaggggcacagcagcacggccaccaaccatggaaacacctgcagctacggcaacgaggagcaggggaagctcatcgggtacgacgccatggcgatggcgagcggcgccgccggcggcgggtaccagctgtcgcagggctcggcgtcgacggtgagcatcgcgagggcgaacggctactcggccaactggagctcgcctttcaatggcgccatgggatga

SEQ ID NO: 187 - DNA, 2088 bp, Oryza sativa
atggccaccatgaacaactggctggccttctccctctccccgcaggatcagctcccgccgtctcagaccaactccactctcatctccgccgccgccaccaccaccaccgccggcgactcctccaccggcgacgtctgcttcaacatcccccaagattggagcatgaggggatcggagctctcggcgctcgtcgccgagccgaagctggaggacttcctcggcggcatctccttctcggagcagcagcatcatcacggcggcaagggcggcgtgatcccgagcagcgccgccgcttgctacgcgagctccggcagcagcgtcggctacctgtaccctcctccaagctcatcctcgctccagttcgccgactccgtcatggtggccacctcctcgcccgtcgtcgcccacgacggcgtcagcggcggcggcatggtgagcgccgccgccgccgcggcggccagtggcaacggcggcattggcctgtccatgatcaagaactggctccggagccagccggcgccgcagccggcgcaggcgctgtctctgtccatgaacatggcggggacgacgacggcgcagggcggcggcgccatggcgctcctcgccggcgcaggggagcgaggccggacgacgcccgcgtcagagagcctgtccacgtcggcgcacggagcgacgacggcgacgatggctggtggtcgcaaggagattaacgaggaaggcagcggcagcgccggcgccgtggttgccgtcggctcggagtcaggcggcagcggcgccgtggtggaggccggcgcggcggcggcggcggcgaggaagtccgtcgacacgttcggccagagaacatcgatctaccgcggcgtgacaaggcatagatggacagggaggtatgaggctcatctttgggacaacagctgcagaagagagggccaaactcgcaagggtcgtcaagtgctgataggtggttatgacaaagaggaaaaagctgctagagcttatgatttggctgctctcaaatactggggcccgacgacgacgacaaattttccggtaaataactatgaaaaggagctggaggagatgaagcacatgacaaggcaggagttcgtagcctctttgagaaggaagagcagtggtttctccagaggtgcatccatttaccgtggagtaactaggcatcaccagcatgggagatggcaagcaaggataggaagagttgcagggaacaaggacctctacttgggcaccttcagcacgcaggaggaggcggcggaggcgtacgacatcgcggcgatcaagttccgggggctcaacgccgtcaccaacttcgacatgagccgctacgacgtcaagagcatcctcgacagcgctgccctccccgtcggcaccgccgccaagcgcctcaaggacgccgaggccgccgccgcctacgacgtcggccgcatcgcctcgcacctcggcggcgacggcgcctacgccgcgcattacggccaccaccaccactcggccgccgccgcctggccgaccatcgcgttccaggcggcggcggcgccgccgccgcacgccgccgggctttaccacccgtacgcgcagccgctgcgtgggtggtgcaagcaggagcaggaccacgccgtgatcgcggcggcgcacagcctgcaggatctccaccacctcaacctcggcgccgccgccgccgcgcatgacttcttctcgcaggcgatgcagcagcagcacggcctcggcagcatcgacaacgcgtcgctcgagcacagcaccggctccaactccgtcgtctacaacggcgacaatggcggcggaggcggcggctacatcatggcgccgatgagcgccgtgtcggccacggccaccgcggtggcgagcagccacgatcacggcggcgacggcgggaagcaggtgcagatggggtacgacagctacctcgtcggcgcagacgcctacggcggcggcggcgccgggaggatgccatcctgggcgatgacgccggcgtcggcgccggccgccacgagcagcagcgacatgaccggagtctgccatggcgcacagctcttcagcgtctggaacgacacataa

SEQ ID NO: 188 - DNA, 2064 bp, Brachypodium distachyon
atggctaccgtgaacaactggctgggcttctccctctccccgcaggagctcccgtcctccgccgctgccgccgccggcgacgtctccggcgccgacgtctgcttcaacatcccgcaagattggggcatgaggggatcggagctgtcggcgctggtggcggagccgaagctggaggacttcctcggcggcatctcttcctactctgaccatcacaaggcagccagaagcaacaacatgaacatcaatggcgccgccgcctgctacgcgagctccggcagcagcggctaccagctttaccacgaccaccccaactccctccagttcgccgactctgtcatggtcgcttcctccgctggcggcgtccacaatgaacatggcatcatggcaagcaccactgccaatggtgcaggcaccaatggcggcatcgggctgtccatgatcaagagctggctgcggagccagccggcgccggcgcagcaggagcagcagagggcggaggggctgtcgctgtccatgaacatgccgctcctgcaggcggcggcggcggagactagcttgtccacgtcggcagcgcagcagggatctcggaaggagaataatggcagcagtagtgctggttctggagccgtggtgtcagccggtactaccagcgccggcgcggtggtggtggagtcgccggcagcggggaggaagaccgccgacacgttcgggcagcggacttcgatctaccgcggcgtcaccaggcatagatggacagggaggtatgaggctcacctgtgggacaacagctgcagaagagaaggacaaactcgcaagggtcgtcaagtttatctaggtggttatgacaaagaggagaaagctgcaagagcttatgatttggctgctctcaagtattggggccccaccacgacgacaaattttccagtagataactacgagaaggagctggaggagatgaagcacatgacaaggcaggagtttgtggcgtctctccgaaggaagagcagtggtttttcaagaggtgcatccatttatcgtggagtaactaggcaccaccaacatgggagatggcaagcaaggatagggagagttgcagggaacaaggatctctacttgggcactttcagcacgcaggaggaggcggcggaggcgtacgacatcgcggcgatcaagttccgggggctgaacgccgtcactaacttcgacatgtcccgctacgacgtcaagagcatcctcgacagcaccgccgcgctgcctgtcggcggcaccaagcgcctcagggacgcggctgccgccgatcagcactaccagcagcgcgccgggggcgtcgtcagctacgccgcgccgcagctcggtggtgtcaacgagaccgcccttgcctatggcgcgccctactaccaccaccaaacctccgccgcagcgtggccgaccatcgcgttccaggcggcgccgcaagcgtcgtcggggcacgggcacatgctgtaccacccgtacgggcagccattgatgcgcgggtggtgcaagcaagagcaggagcaagggcaagggcagcaggagccggaccacgcggtgatcgcggccgcgcacagcctgcaggacctccaccacctcaacctcggcgccggtgcgcacgacttcttctcccagcatgcccacgccatgcaccagcagcagcagcagcacggcggcctcggcagcgtcgacaacaacggcgcggcgtcgctggagcacagcactggctccaactccgtcgtctacaacggggccgctgccgccggggacaccaacaacagctacatgctgccgcccatgagcgctgcagcagcagctggcttcggcctccgcgatcagcaggacgaaggcgggaagatggcgtacgagaacttcctcctcggcgccgccaccgacggctactgtggccctggaaggatggcggccacctggacaccggtgtcggtttcggcggcccagccagtggcggcaacgagcagcggcagcgacatggccggtgccgtctgccatggcggcgcgcagctcttcagcgtctggaacgacgacagttag

SEQ ID NO: 189 - DNA, 1740 bp, Brassica napus
atgaataataactggttaggcttttctctctctccttatgaacaaaatcaccatcgtaaggacgtctgctcttccaccaccacaaccgccgtagatgtcgccggagagtactgttacgatccgaccgctgcctccgatgagtcttcagccatccaaacatcgtttccttctccctttggtgtcgtcctcgatgctttcaccagagacaacaatagtcactcccgagattgggacatcaatggtagtgcatgtaataacatccacaatgatgagcaagatggaccaaaacttgagaatttccttggccgcaccaccacgatttacaacaccaacgaaaacgttggagatatcgatggaagtgggtgttatggaggaggagacggtggtggtggctcactaggactttcgatgataaagacatggctgagaaatcaacccgtggataatgttgataatcaagaaaatggcaatggtgcaaaaggcctgtccctctcaatgaactcatctacttcttgtgataacaacaactacagcagtaacaaccttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaacgagaaggccaaacgcgcaaaggaagacaagtttatttgggaggttatgacaaagaagaaaaagcagctagggcttatgatttagccgcactcaagtattggggaaccaccactactactaacttccccatgagcgaatatgagaaagagatagaagagatgaagcacatgacaaggcaagagtatgttgcctcacttcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaataatgctagcggttggcaaaacgctgcggttcagcatcatcagggagtagatttgagcttattgcagcaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcagatgattcggttactgtttgtggaaatgttgttggttatggtggttatcaaggatttgcagccccggttaactgcgatgcctacgctgctagtgagtttgactataacgcaagaaaccattattactttgctcagcagcagcagacccagcattcgccaggaggagattttcccgcggcaatgacgaataatgttggctctaatatgtattaccatggggaaggtggtggagaagttgctccaacatttacagtttggaacgacaattag

SEQ ID NO: 190 - DNA, 1740 bp, Brassica napus
atgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattag

SEQ ID NO: 191 - DNA, 1731 bp, Brassica rapa
atgaataataactggttaggcttttctctctctccttatgaacaaaatcaccatcgtaaggacgtctgctcttccaccaccacaaccgccgtagatgtcgccggagagtactgttacgatccgaccgctgcctccgatgagtcttcagccatccaaacatcgtttccttctccctttggtgtcgtcctcgatgctttcaccagagacaacaatagtcactcccgagattgggacatcaatggtagtgcatgtaataacatccacaatgatgagcaagatggaccaaagcttgagaatttccttggccgcaccaccacgatttacaacaccaacgaaaacgttggagatatcgatggaagtgggtgttatggaggaggagacggtggtggtggctcactaggactttcgatgataaagacatggctgagaaatcaacccgtggataatgttgataatcaagaaaatggcaatggtgcaaaaggcctgtccctctcaatgaactcatctacttcttgtgataacaacaactacagcagtaacaaccttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaacgagaaggccaaacgcgcaaaggaagacaaggaggttatgacaaagaagaaaaagcagctagggcttatgatttagccgcactcaagtattggagaaccaccactactactaacttccccatgagcgaatatgagaaagagatagaagagatgaagcacatgacaaggcaagagtatgttgcctcacttcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaataatgctagcggttggcaaaacgctgcggttcagcatcatcagggagtagatttgagcttattgcagcaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcagatgattcggttactgtttgtggaaatgttgttggttatggtggttatcaaggatttgcagccccggttaactgcgatgcctacgctgctagtgagtttgactataacgcaagaaaccattattactttgctcagcagcagcagacccagcattcgccaggaggagattttcccgcggcaatgacgaataatgttggctctaatatgtattaccatggggaaggtggtggagaagttgctccaacatttacagtttggaacgacaattag

SEQ ID NO: 192 - DNA, 1704 bp, Brassica rapa
atgaataataattggctaggtttttctctctctccttctgaccaaaatatccatcgtacgggggtcgtcgactcttccaccgccacaacagccgtagatgtcacgggggagtattgttacgatatgactgctgcctctgatgagtcttcagctgttcaaacatcgtttccttctccctttggtgtcgtcctcgatgccttcaccagagacaacaatagtcactcccgagattgggacatcaatggtggtgcatgcaataacatccacagtaatgaccaaaatgaaccaaagcttgagaatttcttcggccgcaccaccacgatttacaacaacagcgaaaatggtggagatggaaatggtggttgtggaggaggagacggtgctggtggctcattaggcctttcgatgattaagacatggctgacgaatcagccggtgtctaatgtggatcatcaagagaataatgggaatgccgcaagaggcttgtcactctctatgaactcatctactacttgtgatagcaacaagtacaacaataggaataatgttgttcaagagaagaccaatgttgatagcgttgatgctacacctaagaaaactattgagagttttggacaaaggacgtctatatatcgcggtgttacaaggcatcggtggacaggaagatatgaggcacatttgtgggataatagttgcaaaagagaaggccaagctcgcaaaggaagacaagtttatttgggaggttatgacaaagaagaaaaagcagctagggcgtatgatttagccgcactcaagtattggggaactaccactactactaatttccctataactgaatatgagaaagaggtagaagatatgaaccacatgacgcggcaagagtatgttgcctctctgcgcaggaaaagtagtggtttctctcgtggagcatcgatttatcgaggagtaacaaggcatcaccaacatggaagatggcaagctaggatcggaagagtcgccggtaacaaagacctctacttgggaacttttgaagaggctgcagaggcatacgacattgctgccatcaaatttagaggattaaccgcagtgactaacttcgaaatgagcagatacaacgttaaagcaatcctcgagagcccgagtcttcctattggtagctctgcgaaacgtctcaaggaagttaaccatccggttcctagtatgatgatcaataataacatttcagagggtgaaaatgatgctagcggttggcaaaacgctgcggttcagcatcatcatggaatagatttgagcttattacagcaacatcaagagaggtacaataattattacaatggagggagtgctagggcttattttaaagaagaggaagatcagcaccatttcttgagcaactcgccgagcctcatggctaatatcgatcagcatagttctgcttcgggtgatatggttactgtttatggaaatgtcgatggttatggtagtcatcaagcacttgcaaccccggttggaacatctcttaactgcgatgctttggccgctgctgagattgcttacgacgcaagaaaccattattattttgctcagcaacagcaaggaggaggaggttttcctgtgggaatttcgaataatgttggctctaatatgtactttaatggggaaggtggtggagaaggtgcgtttacagtttggaacaacacttag

SEQ ID NO: 193 - DNA, 1725 bp, Boechera stricta
atgaactcaatgaataactggttaggtttctctctctctcctcatgatcaaaatcatcaccgtacggacgtcgactccaccaccacaaccgccgtagatgtcgccggagagtactgcttcgatatggccgctgcctccgatgggtcttctgccgttcaaacatcgtttccttctccttttggtgtcgtcctcgaagctttcaccagagacaacaatagtcactcccgagattgggacatcaatagtggtgcatgcaataacatccacaataatgaacaaaatggaccaaagcttgagaatttcctcggccgcaccaccactatttacaataccaacgagaacgatggaagtgtcggttgtagaggaggagacggtggtagtggtggtggctcactaggcctttcaatgataaaaacatggctaagtaatcagccggtggctaatattaatcatcaagacaatggtaatggtgcacgaggcttgtctctctctatgaattcatctactacttgtgatagtaacagctacaacaataataatgatgttgtccaagagaagactattgttgatatcgtagaagctacaccaaagaaaactgttgagagttttggacaaagaacgtctatataccgcggtgttacaaggcaccggtggacaggaagatatgaggcacatttatgggacaatagttgcaaaagagaaggccaaactcgcaaaggaagacaaggaggttatgacaaagaagaaaaagcagctagggcttacgatttagccgcactaaaatattggggaaccaccactactaccaacttccccatgagtagttatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaagaagaggctgcagaggcttatgacattgcagccattaaattcagaggattaagcgcagtgactaactttgacatgagcagatacaatgttaaagcaatactagagagcccgagtctacctattggtagttctgcgaaacgtctcaaggacgttaacagtccggttccaagtatgatgatcagtaataacgtttcagagagtgctaataatgttagcggttggcaaaacgctgcgtttcatcatcatcagggaatggatttgagcttattgcagcaacagcaggagaggtacgttggttattacaatggaggaaacttgtcttccgagagtactagggtttgtttcaaacaagaggaagaaaaacaacatttcttgagtaactcgatcgatcatcatagttcgacttctaatgattcggttactgtttgtgggaatgttgttggttatggtggctatcaaggatttgcaatccctgttggaacatcggttaattgtgattctttcactgctgctgagattgcttacaacgcaagaaaccattattattttgctcagcaacatcaacaacagcagattcagcagtccccggaaggagattttccggcggcgatttcgaataatgttggctctaacatgtacttccatggggaaggtggtggagaaggggcttcaacgtttacagtttggaacgacagttag

SEQ ID NO: 194 - DNA, 1719 bp, Capsella rubella
atgaactcgatgaataactggctaggtttctctctctcccctcatgatcaaaatcatcaccgttcgacggacgtcgactcctccaccaccacaaccgccgtagatgtcgccagtgagtactgcttcgatctggccgctgcctccgatgtgtcttctgccgttcaaacatcgtttccttccccttttggtgtcgtcctcgaagctttcaccagagacaacaatagtcactcccgagattgggacatcaatagtggtgcatgcaataccatccacaataatgagcaaaatggaccaaagcttgagaatttcctcggccgcaccactacgatttacaatacggccaacgagagcgttggagatggcagtggcggtttaggaggaggagatggtggctcactgggcctttcaatgataaaatcatggctgagtaatcagccggtggctaatgttaatcaccaagacaatggtaatggtgcacgaggcttgtctctctctatgaattcatctactacttgtgatagcaaaaactacaacaataatgatgctgtccaagagaagactattgttgatgtcgtagaagctacaccaaagaaaactattgagagttttggacaaagaacctctatataccgcggtgttacaaggcatcggtggacaggaagatatgaggcacatttatgggacaacagttgcaaaagagaaggccaaactcgcaaaggaagacaaggaggttatgacaaagaagaaaaagcagctagggcttacgatttagccgcactaaaatattggggaacgaccactactactaactttcccatgagtagttatgagaaagaggtagaagagatgaagcacatgacgaggcaagagtatgttgcttctctgcgcaggaaaagtagtggtttctctcgtggtgcatcgatttatcgaggcgtaacaaggcaccaccaacatggaaggtggcaagctaggatcggaagagtcgctggtaacaaagacctctacttgggaactttcggcacacaagaagaggctgcagaggcttatgacattgcagccattaaattcagaggactaagcgcagtgactaactttgacatgagcagatacaatgttaaagcaatactcgagagcccaagtctacctatcggtagttctgcgaaacgtctcaaggaagttaacacggttccaagtatggtgatcagtaataacgtttcggagagtgctaataatgttagcggttggcaaaacgctgcgttccaacatcatcagggaatggatttgagcttattgcagcaacagcaggagaggtacgttggttattacaatggaggaaacttgtcttccgagagtactagggtttgtttcaaacaaggtgaagaacagcaacgtttcttgagtaactcgatggatcattatagttcgacttctgatgattcggttacagtctgtggaaatgctgttggttatggtggttatcaaggatttgcaatccctgttggaacatcggttaattgcgatgcttacactgctgctgagattgcttacaacgctagaagccattactattatgctcagcagcagcagcaacaacagcagattcagcagtcgccggaaggagatttcccggtagccattactattggctctaacatgtactttcatggggaaggtggtggagaaggggcttcaacgttttcagtttggaatggtacttag

SEQ ID NO: 195 - DNA, 1728 bp, Capsella grandiflora
atgaactcaatgaataactggctaggtttctctctctcccctcatgatcaaaatcatcaccgttcgacggacgtcgactcctccaccaccacaaccgccgtagatgtcgccagtgagtactgcttcgatctggccgctgcctccgatgtgtcttctgccgttcaaacatcgtttccttccccttttggtgtcgtcctcgaagctttcaccagagacaacaatagtcactcccgagattgggacatcaatagtggtgcatgcaataccatccacaataatgagcaaaatggaccaaagcttgagaatttcctcggccgcaccactacgatttacaatacggccaacgagagcgttggagatggcagtggcggtttaggaggaggagatggtggctcactgggcctttcaatgataaaatcatggctgagtaatcagccggtggctaatgttaatcaccaagacaatggtaatggtgcacgaggcttgtctctctctatgaattcatctactacttgtgatagcaaaaactacaacaataatgatgttgtcaaagagaagactattgttgatgtcgtagaagctacaccaaagaaaactattgagagttttggacaaagaacctctatataccgcggtgttacaaggcatcggtggacaggaagatatgaggcacatttatgggacaacagttgcaaaagagaaggccaaactcgcaaaggaagacaaggaggttatgacaaagaagaaaaagcagctagggcttacgatttagccgcactaaaatattggggaacgaccactactactaacttccccatgagtagttatgagaaagaggtagaagagatgaagcacatgacgaggcaagagtatgttgcttctctgcgcaggaaaagtagtggtttctctcgtggtgcatcgatttatcgaggcgtaactaggcaccaccaacatggaaggtggcaagctagaatcggaagagtcgctggtaacaaagacctctacctgggaactttcggcacacaagaagaggctgcagaggcttatgacattgcagccattaaattcagaggactaagcgcagtgactaactttgacatgagcagatacaatgttaaagcgatcctcgagagcccgagtctacctatcggtagttctgcgaaacgtctcaaagaagttaacagtccggttccaagtatggtgatcagtaataacgtttcggagagtgctaataatgttagcggttggcaaaacgctgcgttccagcatcatcagggaatggatttgagcttattgcagcaacagcaggagaggtacgttggttattacaatggaggaaacttgtcttccgagagtactagggtttgtttcaaacaaggtgaagaacagcaacgtttcttgagtaactcgatggatcatcatagttcgacttccgatgattcggttacagtctgtggaaatgctgttggttatggtggttatcaaggatttgcaatccctgttggaacatcggtgaattgcgatgcttacactgctgctgagattgcttacaacgcaagaagccattactattatgctcagcagcagcagcaacaacagcagattcagcagtcgccggaaggagattttcctgtggcggttttgaataatgttggctctaacatgtactttcatggggaaggtggcggagaaggggcttcaacgttttcagtttggaatggtacttag

SEQ ID NO: 196 - DNA, 2097 bp, Setaria viridis
atggctactgtgaacaactggctggctttctccctctccccgcaggatctgccgccttcccagacggattccaccctcatctccgccaccgccaccgacgaggtctccggcgacgtctgcttcaacatcccccaagattggagcatgaggggatcggaactctcagcgctagtcgccgagccgaagctggaggacttcctcggcggcatcaacttctcggagcagcaccacaaggccaacctcaacgtgatccccagcagtagtaacgcttgctacgcgagctcaggcgctagcaccggctaccaccagctgtaccaccaccaaagctccgcgctccatttcgctgactccgtcatggtggcctcctccgccggcgtccatgacggcggtgccagcatgctcagtgcagcggccaccgtcaatggcggcgctggcgcagccagtgccaacggcggcagcatcgggctgtccatgatcaagaactggctgcggagtcaaccagctccgccgctgccgcagccgagagtagtggcggccgcagagggcgcgcaggcggcgcaggggctttctttgtccatgaacatggcaggggcgcaaggcgctggcatgccgcttctcgccggagagcgaggccgggcgcccgagagcgtgtccacatcagcacagggtggagcagtcgccgctcggaaggaggatagcggtggcagcggcggtgccggcgccctagtagccgtgagcacagacacgggtggcagcggcacggttgctgagacggcggcgaggaagacggtagacacgttcggccagcgcacgtcgatttaccgcggcgtgaccaggcatagatggacagggaggtacgaggctcatctgtgggacaacagttgcagaagggaaggacaaactcgcaagggtcgtcaagtctatctaggtggttatgacaaagaggagaaagctgctagggcttatgatttggctgctcttaagtattggggtcccacgacgaccacaaattttccagtgagtaactacgaaaaggagctggaggaaatgaagcacatgacaaggcaggagtttgtagcgtctctgagaaggaagagcagtggcttctccagaggtgcatccatttacagaggagtaactaggcatcaccaacacgggagatggcaagcacggattggacgagttgcagggaacaaggatctctacttgggcaccttcagcacgcaggaggaggcagcagaagcgtacgacatcgcggcgatcaagttccgcgggctcaacgccgtcaccaacttcgacatgagccgctacgacgtgaagagcatcctggacagcagcgcgctccccatcggcagcgccgccaagcgcctcaaggaggccgaggccgccgcgtccgcgcagcacgccggcgtggtgagctacgacgtaggccgcatcgcctcgcacctcggcgacggcggcgccctggcagcctacggcacgcactaccacgcagccgcagccgcctggccgaccatcgcgttccagccgagcgccgccgccgccggcctgtaccacccgtacgcgcagccgatgcgtgggtggtgcaagcaggagcaggatcacgcggtgatcgccgccgcgcacagcctgcacgagctcaaccacctgaacctgggcgccgccgccggtgcgcacgacttcttctcggcggggcaggccgcgatgcacggcctcggcagcatcgacaacgcctcactcgagcatagcaccggctccaactccgtcgtgtacaatggggttggagacagcaacggtggcgccgtcggcggcggctacatgatgccgatgagcgctgccgcggcgaccacgacggcaatggttagtcacgagcaggtgcatgcgcggtcacatcaaggggagcacgacgaagccaccaagcaggcggcgcagatggggtacgagagctacctggtgaacgcggaggcggcctacggtggcgggaggatgccttcgtggacgccggcctcggccccggcagcggcaagcagcaacgacaacatggccggcgttggccatggcggcgcgcagctcttcagtgtctggaatgacacataa

SEQ ID NO: 197 - DNA, 2133 bp, Zea mays
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 198 - DNA, 2121 bp, Zea mays
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 199 - DNA, 1629 bp, Pennisetum squamulatum
atgggttccaccaacaactggctgcgcttcgcctcgttctccggcggcggcggcgccaaggatgccgcggccctgctcccgctgccgccctcgccccgtggcgatgtcgacgaggccggcgcagagccgaagctcgaggacttcctcggcctgcaggagccgagcgccgccgcggtgggggctgggcggccattcgcggtgggtggcggtgcgagctccatcgggctgtccatgatcaggaactggctgcgcagccagccggcgccggccgggcctgctgcgggggtcgattcgatggtgctggcggctgcggcggcgtcgacggaggtggccggcgatggcgcggagggcggcggcgccgtggctgacgcggtgcagcagaggaaggcggcggcggtggacactttcgggcagcggacctccatataccgcggcgtcacaaagcatagatggacaggaaggtatgaagcccatctttgggacaatagctgcagaagagaaggtcaaactcggaaaggtagacaaggtggatatgataaagaagaaaaagcagctagagcttatgatttagctgctctcaagtaccggggcaccacaactactacaaattttccgatgagcaactatgaaaaggagttagaagagatgaagcatatgtcacgacaagaatatgttgcatcccttagaaggaaaagcagtggtttttctcgtggtgcatcaatttaccgaggggttaccaggcaccatcagcatggaaggtggcaagcaagaataggaagtgtggcaggaaacaaggatctttatttgggcacattcagtacccaggaggaagctgcagaggcttacgacattgctgccatcaaattccgaggcctcaatgctgtcacgaactttgacatgagccggtatgacgtcaagagcatcattgagagcagctccctgcctgttggcggcactccaaagcgtctcaaggaagtgcctgatcaatcagatatgggcatcaacataaacggtgactctgctggtcatatgactgctatcaaccttcttactgatggcaatgacagctatggagctgagagttatggttacagtggttggtgtcccacagccatgacgccaatcccctttcaattcagcaatggccatgaccattccaggctgtggtgcaagccagagcaggacaatgcggttgttgcagcactgcataacctgcatcacctccagcacttgccagccccagttggcacccataattttttccagccatcgcctgttcaggacatgacaggtgttgccgatgcttcatcgccaccagtagaatctaattcattcctgtacaatggggacgttggttaccatggtgccatgggtggcagctatgccatgccggttgccacactagttgagggcaactctgcgggcagtggctatggagttgaggaaggcacagggtctgaaatctttggtggacggaacttgtattctctctcccaaggttcctcaggcgccaatactggaaaggcagatgcttatgaaagctgggatccatctatgctggtgatatcacagaagtctgccaatgtgactgtctgccatggcgcacctgtattttcagtttggaaatga

SEQ ID NO: 200 - DNA, 1755 bp, Arabidopsis thaliana
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaatacattaaccaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggacccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaacaatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 201 - DNA, 1659 bp, Setaria italica
atgggttccaccaacaactggctgggcttcgcctcgttctccggcgccgcggatgacgccgcaatcctgcccccgctgccgccgtcgccccgtggcgatggggccggagcggagccgaagctggaggacttcctcggcctgcaggaaccggccgccacggtggctgccggacggccgttcgtgggtaccggcggcgcgagctccattgggctgtccatgatcaagaactggctgcgaagccagccggcgccggagcctgctgtggcggccgattcgatggcgctggcggcggtggcggtggtgtctccggaggggagcggcaaggtgaccgacgacggcgctgagagcggcggaggcgccgtggtggttgcggcgcagcagaggaaggcggcggcggtggacacgttcggccagcggacgtccatctaccgcggcgtcacaaagcatcgatggacaggaagatatgaagcacatctttgggacaacagctgcagaagagaaggccagactcgcaaaggaagacaagtgtatcttggtggatatgataaagaagagaaagcggccagagcttatgatctagctgctctcaagtactggggcactacaacaactacaaattttcagatcagcaactacgaaaaagagttggaagagatgaagcatatgtcacggcaggaatatgttgcatcccttagaaggaaaagcagcgggttttctcgtggtgcatctatttaccgaggggttaccaggcaccatcagcatggaaggtggcaagcaagaataggaagagtggcaggaaacaaggatctgtatttgggcacattcagtactcaggaggaagctgcagaggcttacgacattgctgcaatcaaattccgaggccttaatgccgtcacaaactttgacatgagccggtacgacgtcaagagcatcatcgagagcagctccctgccagtaggtggcaccacaaagcgtctcaaggatgtgcctgatcaatctgacatgggcaggaacggccacagtgcagactctgttggtcatatgactgctaccaaccttcttactgatggcattggcagctatggccctgagaattatggttatagtggatggtcgcccgctgccatgacatcaatccccttgcaattcagcaatggccatgaccagtccaggctgtggtgcaagccagagcaggacagtgcggttgttgcagcagcacacaacctgcaccacctccagcactttccggccccaggtggcacccacaattttttccagccatcgcccattcaggacatgacaggtgttgctgatgtttcatctccatcagtggactcgaattcattctcgtacaatgggagcgttggttaccatggtgccatgggtggcggctatgccatgccggttacaacactagtggagggcaaccctgcagccagtggctatggagttgaggaaggcacgactgacgtctatgattgtcggaacatttattatctctcccaaggctcgccaggtgccaatactggaaagccagaagcttatgatcagcaaggtgccgggtatgaaagttgggtaccggctgtgccggtgatatcgcagaaggctgccaatgtgactgtctgccatggcacacctctatactcggtttggaaatga

SEQ ID NO: 202 - DNA, 1662 bp, Panicum virgatum
atgggttccaccaacaactggctgggcttcgcctcgttctccggtgccgccgcagccgacgatatcctgcccccgctgccgccgccccgaggcgatgaggctgccgcggagccgaagctcgaggacttcctcggcctgcaggagccggcggccggagtggccggacgggcgccgttcgcggggagcggcggcggcgcgagctccatcgggctgtccatgatcaagaactggctgcgcagccagccggcgccgggacctgcgggggccgattcgatggctctggcggtggtggaagaagcgtcgacggacgaggtcaggaaggtgaccgacgaccgcggcgcggagagcgttgccgccgtggtcgacgcggcgcagcagaggaaggcggtggctgcggtggatacgttcgggcagcggacctccatataccgcggcgttaccaagcatagatggacaggaaggtatgaagcccatctttgggacaacagctgcagaagagaaggtcagactcgcaagggcagacaagtgtatcttggtggatatgataaagaagagaaagccgccagggcttatgatctagctgctctcaagtactggggcactacaacgactacaaattttccgatgagcaactacgaaaaagagttagaagagatgaagcatatgtcacgacaagaatatgttgcatcccttagaaggaaaagcagcggattttctcgtggtgcgtctatttaccgaggggttacaaggcaccatcagcatggaaggtggcaagcaagaataggaagggtggcaggaaacaaggatctgtatttgggcacattcagtacccaggaggaagcagcagaggcttacgacatcgccgcaatcaaattccgaggccttaatgcggtcactaactttgacatgagccggtatgacgtcaagagcatcatggagagcagcgccctaccagttggtggcaccacaaagtgtctcaaggatgtgcatgatcaatctgatatgggcatgaacagcagcggtgcagactctgccagccatatgactgctactacaaagcttcttactgatggcattggtagctacggcaatgagaattatggttacagtggctggtcgccctctgccatgatgcgtatccccctgcaattcagcaatggccaggaacactccaggctgtggtgcaagccagagcaggacagtgcggttgttgccgcagcacacaacctgcagcatctccagcacttcccgtccccaggtggcactcacgatttctttcatccatcacatgttcaggacgtgacgggcgtcgctgatgtttcatcaccatcagtggaccctaattcattcttgtacaatggagttgttggttaccatggttccatgggtggcggttatgccatgcctgtcgccaccctagtggatagcaaccatgcaaccagtagctatggagttgaggaaggcacttctgaattatatagtggtcagaacttgtattatctctcccaagcttccccaggcgccaatactggaaaggcagatgcttatgagcagcaaggtgttgggtatgaaagctgggttccatctgtgcctgtgatatcgcagaaggaccccaatgtgacggtttgccatggcacacctctattctcagtttggaagtga

SEQ ID NO: 203 - DNA, 1584 bp, Panicum hallii
atgggttccacgaacaactggctgggcttcgcctcgttctccggcgccgccgcagccgacgatatcctgcccccgctgccgccgccccgaggcgatgaggcggacgctgagccgaagctcgaggacttcctcggcctgcaggggccggccgcagcagtggccggacggccgttcgcggggagcggcggcggcgcgagctccatcgggctgtccatgatcaagaactggctgcgcagccagccggcgccgggacctacgggggcagattcgatggctctggcggcggtggaagcggcgtcgacggacgggagcgggaaggtggccgacggcgcggtgagcgacgccgccgtggtggacgcggcgcagcagaggaaggcggcggcggcggtggacacgttcgggcagcggacctccatataccgcggcgtcacgaagcatagatggacaggaaggtatgaagcccatctttgggacaacagctgcagaagagaaggtcaaactcgcaagggcagacaagtgtatcttggtggatatgataaagaagagaaagcagccagggcttatgatctagctgctctcaagtactggggcactacaacgactacaaattttccgatgagcaactacgagaatgagttagaagagatgaagcatatgtcacgacaagaatatgttgcatcccttagaaggaaaagcagcggattttctcgtggtgcgtcgatttaccgaggggttacaaggcaccatcagcatggaaggtggcaagcaagaataggaagggtggcaggaaacaaggatctgtatttgggcacattcagtacccaggaggaagcagcagaggcttacgacattgccgcgatcaaattccgaggccttaatgcggttacaaactttgacatgagccggtataacgtcaagagcatcatggagagcagcgccctaccagttggtggcaccacaaagcgtctcaaggatgtgcctgatcaatcagatatgggcatgaacagcaacgttgcagactctgctggccatatgactgctacaaagcttcttactgatggcattggcagctatggccatgagaattatggttacagtggctggtcggcctctgccatgacgccaatccccctgcaattcagcaattgccatgaccactccaggctgtggtgcaagccagagcatgacagtgcggttgttgcggcagcacacaacctgcaccacctccagcacttccctgacccaggtggcactcacaattttttccatccatcgcctggtcaggacgtgaccggtgtcaccaatgtttcatcaccatcagtggactctaattcattcttgtacagtggagatgttggttaccatggtgccatgggtggtggttatgccatgcctgttgccacactagttgatagcaaccatgcggccagtggctatggagttgaggaaggcacatctgacttatatagtggtcggaatttgtattatctctcccaagcttccccaggcaccaatactggaaaggcagatgcttatgaacagcaaggtgttgggtatgaaagctgggttccatctgtgcggtga

SEQ ID NO: 204 - DNA, 2139 bp, Panicum hallii
atggctactgtgaacaactggctggctttctccctctccccgcaggatctgccgccttcccagacggactccaccctcatctccgccgccgccaccgacgaggtctccggcgatgtctgcttcaacatcccccaagattggggcatgaggggatcggagctttcagctctcgtctccgagcccaagctggaggacttcctcggcggcatcaacttctcggagcagcatcacaaggccaacctcaacgtgatccccagcagtagcagcacttgctacgcgagctcaggtgctagcaccggctaccaccaccagctgtaccaccaccccagctccgcgctccatttcgctgactccgtcatggtggcctcttccgccggcgtccatgacggcggtgccatgctcagcgcggccgccgccaatggtggagctggcgctgccggtgccaatggcggcagcatcgggctgtccatgatcaagaactggctgcggagccagccagctccgccgccgcagccgagagtggcggtggccgagggcgcgcaggcggcgcagggtctctctttgtccatgaacatggcagggacgcaaggagctggcatgccgcttctcgccggagagcgaggccgggcgcccgagagcgtatccacgtcggcacagggtggagccgtcgccgctcggaaggaggacagcggtggtgccggcgccctagtagcagtgagcacggacacgggtggcagcggcggcgcgtctgctgagacggtggcgaggaagacggtggacacgttcggccagcgcacgtcgatttaccgcggcgtgaccaggcacagatggacggggaggtacgaggctcacctgtgggacaacagttgcagaagggaaggacaaactcgcaagggtcgtcaaggtggttatgacaaagaggagaaagctgccagggcttatgatttggctgctcttaagtactggggtcccacgacgacgacaaattttccggtgagtaactacgaaaaggagttggaggaaatgaagcacatgacaaggcaggagtttgttgcgtctctgagaaggaagagcagtggcttctccagaggtgcatccatttacagaggagtaactaggcatcaccaacatgggaggtggcaagcacggattggacgagttgcagggaacaaggacctctacttgggcaccttcagcacgcaggaggaggcggcggaggcgtacgacatcgcggcgatcaagttccgcggcctcaacgccgtcaccaacttcgacatgagccgctacgacgtgaagagcatcctggacagcagcgcgctccccatcggcagcgctgccaagcgcctcaaggaggccgaggccgccgcgtccgcgcagcatcatgccggcgtggtgagctacgacgtcggccgcatcgcctcgcagctcggcgacggcggcgccctggcggcctacggagcgcactatcacgccgccgccgccgccgcttggccgaccatcgcgttccagccgggcgccaccgccggcctgtaccatccgtatgcgcagccgctgccacgcgggtggtgcaagaaggagcaggaccacgcggtgatcgcggccgcgcacagcctgcaggagctcaaccacctgaacctgggcgccggcgcgcacgacttcttctcggcggggcaggcggcgatgcacggcctcggcagcatcgacaactcctctctggagcatagcaccggctccaactccgtcgtgtacaacggggtaggggacagcaacggtggcgccgtaggcggcggctacatgatgaccatgagcgctgccgcggcgaccaccacggcgatggtgagccacgagcaggtgcatgcacgggcacaaggggaccacgacgaagccagcaaacatgcggcgcagatggggtacgagagctacctgatgaacgcagaggcggcctacggcggcgggaggatgccttcctggacgcggcctcggctccaccggtggcggcggcggcaagcagcaacgacaacatggccggcgttggccatggcggcgcgcagctcttcagtgtctggaatgacacttaataagctgcgtacgccgacccggcatcgtggcaaaaagtacgctgacataagtaacacttaa

SEQ ID NO: 205 - protein, 698 aa, Setaria italica
MATVNNWLAFSLSPQDLPPSQTDSTLISATATDEVSGDVCFNIPQDWSMRGSELSALVAEPKLEDFLGGINFSEQHHKANLNVIPSSSNACYASSGASTGYHQLYHHQSSALHFADSVMVASSAGVHDGGASMLSAAATVNGGAGAASANGGSIGLSMIKNWLRSQPAPPLPQPRVVAAAEGAQAAQGLSLSMNMAGAQGAGMPLLAGERGRAPESVSTSAQGGAVAARKEDSGSSGGAGALLAVSTDTGGSGTVAETAARKTVDTFGQRTSIYRGVTRHRWTGRYEAHLWDNSCRREGQTRKGRQVYLGGYDKEEKAARAYDLAALKYWGPTTTTNFPVSNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHAGVVSYDVGRIASHLGDGGALAAYGTHYHAAAAAWPTIAFQPSAAAAGLYHPYAQPMRGWCKQEQDHAVIAAAHSLHELNHLNLGAGGGAHDFFSAGQAAMHGLGSIDNASLEHSTGSNSVVYNGVGDSNGGAVGGGYMMPMSAATATTTAMVSHEQVHARSHQGEHDEATKQAAQMGYESYLVNAEAAYGGGRMPSWTPASAPAAASSNDNMAGVGHGGAQLFSVWNDT

SEQ ID NO: 206 - protein, 693 aa, Panicum virgatum
MATVNNWLAFSLSQQDLPPSQTDSTLISAAATDEVSGDVCFNIPQDWSMRGSELSALVAEPKLEDFLGGINFSEQHHKANLNVIPSSSSTCYASSGASTGYHQLYHHPSSALHFADSVMVASSAGVHDGGAMLSAATAHGGAGAAGANGGSIGLSMIKNWLRSQPAPPPQPRVAVPEGAQAVQGLSLSMNMAGTQGAGMPFLAGDRGRAPESVSTSAQGGAVAARKEDSGGSGGAGALVVAVSTDTGGSGSGGASAETAARKTVDTFGQRTSIYRGVTRHRWTGRYEAHLWDNSCRREGQTRKGRQGGYDKEEKAARAYDLAALKYWGPTTTTNFPVSNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHAGVVSYDVGRIASQLGDGGALAAYGAHYHAAAAAAWPTIAFQPGAAAGGLYHPYAQPLRGWCKQEQDHAVIAAAHSLQELNHLNLGAGAHDFFSAGQAAMHGLGSIDNSSLEHSTGSNSVVYNGVGDSNGGGGYMMPMNAAAATTTAMVNHEQVHARAHGDHDEASKQVMGYESYLVNAEAAYGGGRMPSWTTASASPVAAAAASSNDNMAGVGHGGAQLFSVWNDT

SEQ ID NO: 207 - protein, 666 aa, Panicum virgatum
MATVNNWLAFSLSPQDLPPSQTDSTLISAAATDEVSGDVCFNIPQDWGMRGSELSALVSEPKLEDFLGGINFSEQHHKANLNVIPSSSSTCYASSGASSAGYHQLYHHPSSALHFADSVMVASSAGVHDGGAMISAATASGGAGAAGANGGSIGLSMIKNWLRSQPAPAPPQPRVAVAEGAQAAQGLSLSMNMAGTQGAGMPFLAGERGRAPESASTSAQGGAVAARKEDSGGSGGAGALVTVSTDTGGSGGASAETAARKTVDTFGQRTSIYRGVTRHRWTGRYEAHLWDNSCRREGQTRKGRQVYLGGYDKEEKAARAYDLAALKYWGPTTTTNFPVSNYEKELEEMKHMTRQEFVASLRSGIFKSDPMSGTQEEAAEAYDIAAIKFRGLNAVTNFDMSRYDVKSILDSSALPIGSAAKRLKEAEAAASAQHHAGVVSYDVGRIASQLGDGGALAAYGAHYHAAAAAWPTIAFQPGAAAAGLYHPYAQPLRGWCKQEQDYAVIAAAHSLQELNHLNLGAGAHDFFSAGQAAMHGLGSIDNSSLEHSTGSNSVVYNGVGDSNGGAVGGGYMMPTSAAAAATTTAMVSHEQVHARAQGDHDEASKRAAHMGYESYLVNAEAAYGGGRMPSWTTASAPPVAAAASSNDNMAGVGHGGAQLFSVWNDT

SEQ ID NO: 208 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVNAGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 209 - protein, 700 aa, Oryza sativa
MASANNWLGFSLSGQENPQPHQDSSPPAAIDVSGAGDFYGLPTSQPTAADAHLGVAGHHHNASYGIMEAFNRGAQEAQDWNMRGLDYNGGASELSMLVGSSGGKRAAAVEETEPKLEDFLGGNSFVSEQDHHAAGGFLFSGVPMASSTNSNSGSNTMELSMIKTWLRNNGQVPAGHQPQQQQPAAAAAAAQQQAHEAAEMSTDASASSFGCSSDAMGRSNNGGAVSAAAGGTSSQSLALSMSTGSHSHLPIVVAGGGNASGGAAESTSSENKRASGAMDSPGGGAIEAVPRKSIDTFGQRTSIYRGVTRHRWTGRYEAHLWDNSCRREGQSRKGRQGGYDKEDKAARAYDLAALKYWGTTTTTNFPISNYEKELDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKEAADHAEAAGATIWRAADMDGAGVISGLADVGMGAYAASYHHHHHHGWPTIAFQQPPPLAVHYPYGQAPAAPSRGWCKPEQDAAVAAAAHSLQDLQQLHLGSAAAHNFFQASSSSTVYNGGGGGYQGLGGNAFLMPASTVVADQGHSSTATNHGNTCSYGNEEQGKLIGYDAMAMASGAAGGGYQLSQGSASTVSIARANGYSANWSSPFNGAMG

SEQ ID NO: 210 - protein, 695 aa, Oryza sativa
MATMNNWLAFSLSPQDQLPPSQTNSTLISAAATTTTAGDSSTGDVCFNIPQDWSMRGSELSALVAEPKLEDFLGGISFSEQQHHHGGKGGVIPSSAAACYASSGSSVGYLYPPPSSSSLQFADSVMVATSSPVVAHDGVSGGGMVSAAAAAAASGNGGIGLSMIKNWLRSQPAPQPAQALSLSMNMAGTTTAQGGGAMALLAGAGERGRTTPASESLSTSAHGATTATMAGGRKEINEEGSGSAGAVVAVGSESGGSGAVVEAGAAAAAARKSVDTFGQRTSIYRGVTRHRWTGRYEAHLWDNSCRREGQTRKGRQVLIGGYDKEEKAARAYDLAALKYWGPTTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSAALPVGTAAKRLKDAEAAAAYDVGRIASHLGGDGAYAAHYGHHHHSAAAAWPTIAFQAAAAPPPHAAGLYHPYAQPLRGWCKQEQDHAVIAAAHSLQDLHHLNLGAAAAAHDFFSQAMQQQHGLGSIDNASLEHSTGSNSVVYNGDNGGGGGGYIMAPMSAVSATATAVASSHDHGGDGGKQVQMGYDSYLVGADAYGGGGAGRMPSWAMTPASAPAATSSSDMTGVCHGAQLFSVWNDT

SEQ ID NO: 211 - protein, 687 aa, Brachypodium distachyon
MATVNNWLGFSLSPQELPSSAAAAAGDVSGADVCFNIPQDWGMRGSELSALVAEPKLEDFLGGISSYSDHHKAARSNNMNINGAAACYASSGSSGYQLYHDHPNSLQFADSVMVASSAGGVHNEHGIMASTTANGAGTNGGIGLSMIKSWLRSQPAPAQQEQQRAEGLSLSMNMPLLQAAAAETSLSTSAAQQGSRKENNGSSSAGSGAVVSAGTTSAGAVVVESPAAGRKTADTFGQRTSIYRGVTRHRWTGRYEAHLWDNSCRREGQTRKGRQVYLGGYDKEEKAARAYDLAALKYWGPTTTTNFPVDNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSTAALPVGGTKRLRDAAAADQHYQQRAGGVVSYAAPQLGGVNETALAYGAPYYHHQTSAAAWPTIAFQAAPQASSGHGHMLYHPYGQPLMRGWCKQEQEQGQGQQEPDHAVIAAAHSLQDLHHLNLGAGAHDFFSQHAHAMHQQQQQHGGLGSVDNNGAASLEHSTGSNSVVYNGAAAAGDTNNSYMLPPMSAAAAAGFGLRDQQDEGGKMAYENFLLGAATDGYCGPGRMAATWTPVSVSAAQPVAATSSGSDMAGAVCHGGAQLFSVWNDDS

SEQ ID NO: 212 - protein, 579 aa, Brassica napus
MNNNWLGFSLSPYEQNHHRKDVCSSTTTTAVDVAGEYCYDPTAASDESSAIQTSFPSPFGVVLDAFTRDNNSHSRDWDINGSACNNIHNDEQDGPKLENFLGRTTTIYNTNENVGDIDGSGCYGGGDGGGGSLGLSMIKTWLRNQPVDNVDNQENGNGAKGLSLSMNSSTSCDNNNYSSNNLVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEIEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENNASGWQNAAVQHHQGVDLSLLQQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQHSPGGDFPAAMTNNVGSNMYYHGEGGGEVAPTFTVWNDN

SEQ ID NO: 213 - protein, 579 aa, Brassica napus
MNNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 214 - protein, 575 aa, Brassica rapa
MNNNWLGFSLSPYEQNHHRKDVCSSTTTTAVDVAGEYCYDPTAASDESSAIQTSFPSPFGVVLDAFTRDNNSHSRDWDINGSACNNIHNDEQDGPKLENFLGRTTTIYNTNENVGDIDGSGCYGGGDGGGGSLGLSMIKTWLRNQPVDNVDNQENGNGAKGLSLSMNSSTSCDNNNYSSNNLVAQGKTIDDSVEATPKKTIESFGQRTSIYRGVTRHRWTGRYEAHLWDNSCKREGQTRKGRQGGYDKEEKAARAYDLAALKYWRTTTTTNFPMSEYEKEIEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENNASGWQNAAVQHHQGVDLSLLQQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQHSPGGDFPAAMTNNVGSNMYYHGEGGGEVAPTFTVWND

SEQ ID NO: 215 - protein, 567 aa, Brassica rapa
MNNNWLGFSLSPSDQNIHRTGVVDSSTATTAVDVTGEYCYDMTAASDESSAVQTSFPSPFGVVLDAFTRDNNSHSRDWDINGGACNNIHSNDQNEPKLENFFGRTTTIYNNSENGGDGNGGCGGGDGAGGSLGLSMIKTWLTNQPVSNVDHQENNGNAARGLSLSMNSSTTCDSNKYNNRNNVVQEKTNVDSVDATPKKTIESFGQRTSIYRGVTRHRWTGRYEAHLWDNSCKREGQARKGRQVYLGGYDKEEKAARAYDLAALKYWGTTTTTNFPITEYEKEVEDMNHMTRQEYVASLRRKSSGFSRGASIYRGVTRHHQHGRWQARIGRVAGNKDLYLGTFEEAAEAYDIAAIKFRGLTAVTNFEMSRYNVKAILESPSLPIGSSAKRLKEVNHPVPSMMINNNISEGENDASGWQNAAVQHHHGIDLSLLQQHQERYNNYYNGGSARAYFKEEEDQHHFLSNSPSLMANIDQHSSASGDMVTVYGNVDGYGSHQALATPVGTSLNCDALAAAEIAYDARNHYYFAQQQQGGGGFPVGISNNVGSNMYFNGEGGGEGAFTVWNNT

SEQ ID NO: 216 - protein, 574 aa, Boechera stricta
MNSMNNWLGFSLSPHDQNHHRTDVDSTTTTAVDVAGEYCFDMAAASDGSSAVQTSFPSPFGVVLEAFTRDNNSHSRDWDINSGACNNIHNNEQNGPKLENFLGRTTTIYNTNENDGSVGCRGGDGGSGGGSLGLSMIKTWLSNQPVANINHQDNGNGARGLSLSMNSSTTCDSNSYNNNNDVVQEKTIVDIVEATPKKTVESFGQRTSIYRGVTRHRWTGRYEAHLWDNSCKREGQTRKGRQGGYDKEEKAARAYDLAALKYWGTTTTTNFPMSSYEKEVEEMKHMTRQEYVASLRRKSSGFSRGASIYRGVTRHHQHGRWQARIGRVAGNKDLYLGTFGTQEEAAEAYDIAAIKFRGLSAVTNFDMSRYNVKAILESPSLPIGSSAKRLKDVNSPVPSMMISNNVSESANNVSGWQNAAFHHHQGMDLSLLQQQQERYVGYYNGGNLSSESTRVCFKQEEEKQHFLSNSIDHHSSTSNDSVTVCGNVVGYGGYQGFAIPVGTSVNCDSFTAAEIAYNARNHYYFAQQHQQQQIQQSPEGDFPAAISNNVGSNMYFHGEGGGEGASTFTVWNDS

SEQ ID NO: 217 - protein, 572 aa, Capsella rubella
MNSMNNWLGFSLSPHDQNHHRSTDVDSSTTTTAVDVASEYCFDLAAASDVSSAVQTSFPSPFGVVLEAFTRDNNSHSRDWDINSGACNTIHNNEQNGPKLENFLGRTTTIYNTANESVGDGSGGLGGGDGGSLGLSMIKSWLSNQPVANVNHQDNGNGARGLSLSMNSSTTCDSKNYNNNDAVQEKTIVDVVEATPKKTIESFGQRTSIYRGVTRHRWTGRYEAHLWDNSCKREGQTRKGRQGGYDKEEKAARAYDLAALKYWGTTTTTNFPMSSYEKEVEEMKHMTRQEYVASLRRKSSGFSRGASIYRGVTRHHQHGRWQARIGRVAGNKDLYLGTFGTQEEAAEAYDIAAIKFRGLSAVTNFDMSRYNVKAILESPSLPIGSSAKRLKEVNTVPSMVISNNVSESANNVSGWQNAAFQHHQGMDLSLLQQQQERYVGYYNGGNLSSESTRVCFKQGEEQQRFLSNSMDHYSSTSDDSVTVCGNAVGYGGYQGFAIPVGTSVNCDAYTAAEIAYNARSHYYYAQQQQQQQQIQQSPEGDFPVAITIGSNMYFHGEGGGEGASTFSVWNGT

SEQ ID NO: 218 - protein, 575 aa, Capsella grandiflora
MNSMNNWLGFSLSPHDQNHHRSTDVDSSTTTTAVDVASEYCFDLAAASDVSSAVQTSFPSPFGVVLEAFTRDNNSHSRDWDINSGACNTIHNNEQNGPKLENFLGRTTTIYNTANESVGDGSGGLGGGDGGSLGLSMIKSWLSNQPVANVNHQDNGNGARGLSLSMNSSTTCDSKNYNNNDVVKEKTIVDVVEATPKKTIESFGQRTSIYRGVTRHRWTGRYEAHLWDNSCKREGQTRKGRQGGYDKEEKAARAYDLAALKYWGTTTTTNFPMSSYEKEVEEMKHMTRQEYVASLRRKSSGFSRGASIYRGVTRHHQHGRWQARIGRVAGNKDLYLGTFGTQEEAAEAYDIAAIKFRGLSAVTNFDMSRYNVKAILESPSLPIGSSAKRLKEVNSPVPSMVISNNVSESANNVSGWQNAAFQHHQGMDLSLLQQQQERYVGYYNGGNLSSESTRVCFKQGEEQQRFLSNSMDHHSSTSDDSVTVCGNAVGYGGYQGFAIPVGTSVNCDAYTAAEIAYNARSHYYYAQQQQQQQQIQQSPEGDFPVAVLNNVGSNMYFHGEGGGEGASTFSVWNGT

SEQ ID NO: 219 - protein, 698 aa, Setaria viridis
MATVNNWLAFSLSPQDLPPSQTDSTLISATATDEVSGDVCFNIPQDWSMRGSELSALVAEPKLEDFLGGINFSEQHHKANLNVIPSSSNACYASSGASTGYHQLYHHQSSALHFADSVMVASSAGVHDGGASMLSAAATVNGGAGAASANGGSIGLSMIKNWLRSQPAPPLPQPRVVAAAEGAQAAQGLSLSMNMAGAQGAGMPLLAGERGRAPESVSTSAQGGAVAARKEDSGGSGGAGALVAVSTDTGGSGTVAETAARKTVDTFGQRTSIYRGVTRHRWTGRYEAHLWDNSCRREGQTRKGRQVYLGGYDKEEKAARAYDLAALKYWGPTTTTNFPVSNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHAGVVSYDVGRIASHLGDGGALAAYGTHYHAAAAAWPTIAFQPSAAAAGLYHPYAQPMRGWCKQEQDHAVIAAAHSLHELNHLNLGAAAGAHDFFSAGQAAMHGLGSIDNASLEHSTGSNSVVYNGVGDSNGGAVGGGYMMPMSAAAATTTAMVSHEQVHARSHQGEHDEATKQAAQMGYESYLVNAEAAYGGGRMPSWTPASAPAAASSNDNMAGVGHGGAQLFSVWNDT

SEQ ID NO: 220 - protein, 710 aa, Zea mays
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGHGGAQLFSVWNDT

SEQ ID NO: 221 - protein, 706 aa, Zea mays
MATVNNWLAFSLSPQELPPSQTTDSTLISAATADHVSGDVCFNIPQDWSMRGSELSALVAEPKLEDFLGGISFSEQHHKSNCNLIPSTSSTVCYASSAASTGYHHQLYQPTSSALHFADSVMVASSAGVHDGGSMLSAAAANGVAGAASANGGGIGLSMIKNWLRSQPAPMQPRAAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAATTGLYHPYAQQPMRGGGWCKQEQDHAVIAAAHSLQDLHHLNLGAAGAHDFFSAGQQAAAAAAMHGLASIDSASLEHSTGSNSVVYNGGVGDSNGASAVGSGGGYMMPMSAAGATTTSAMVSHEQMHARAYDEAKQAAQMGYESYLVNAENNGGGRMSAWGTVVSAAAAAAASSNDNIAADVGHGGAQLFSVWNDT

SEQ ID NO: 222 - protein, 542 aa, Pennisetum squamulatum
MGSTNNWLRFASFSGGGGAKDAAALLPLPPSPRGDVDEAGAEPKLEDFLGLQEPSAAAVGAGRPFAVGGGASSIGLSMIRNWLRSQPAPAGPAAGVDSMVLAAAAASTEVAGDGAEGGGAVADAVQQRKAAAVDTFGQRTSIYRGVTKHRWTGRYEAHLWDNSCRREGQTRKGRQGGYDKEEKAARAYDLAALKYRGTTTTTNFPMSNYEKELEEMKHMSRQEYVASLRRKSSGFSRGASIYRGVTRHHQHGRWQARIGSVAGNKDLYLGTFSTQEEAAEAYDIAAIKFRGLNAVTNFDMSRYDVKSIIESSSLPVGGTPKRLKEVPDQSDMGININGDSAGHMTAINLLTDGNDSYGAESYGYSGWCPTAMTPIPFQFSNGHDHSRLWCKPEQDNAVVAALHNLHHLQHLPAPVGTHNFFQPSPVQDMTGVADASSPPVESNSFLYNGDVGYHGAMGGSYAMPVATLVEGNSAGSGYGVEEGTGSEIFGGRNLYSLSQGSSGANTGKADAYESWDPSMLVISQKSANVTVCHGAPVFSVWK

SEQ ID NO: 223 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNTLTNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGPTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 224 - protein, 552 aa, Setaria italica
MGSTNNWLGFASFSGAADDAAILPPLPPSPRGDGAGAEPKLEDFLGLQEPAATVAAGRPFVGTGGASSIGLSMIKNWLRSQPAPEPAVAADSMALAAVAVVSPEGSGKVTDDGAESGGGAVVVAAQQRKAAAVDTFGQRTSIYRGVTKHRWTGRYEAHLWDNSCRREGQTRKGRQVYLGGYDKEEKAARAYDLAALKYWGTTTTTNFQISNYEKELEEMKHMSRQEYVASLRRKSSGFSRGASIYRGVTRHHQHGRWQARIGRVAGNKDLYLGTFSTQEEAAEAYDIAAIKFRGLNAVTNFDMSRYDVKSIIESSSLPVGGTTKRLKDVPDQSDMGRNGHSADSVGHMTATNLLTDGIGSYGPENYGYSGWSPAAMTSIPLQFSNGHDQSRLWCKPEQDSAVVAAAHNLHHLQHFPAPGGTHNFFQPSPIQDMTGVADVSSPSVDSNSFSYNGSVGYHGAMGGGYAMPVTTLVEGNPAASGYGVEEGTTDVYDCRNIYYLSQGSPGANTGKPEAYDQQGAGYESWVPAVPVISQKAANVTVCHGTPLYSVWK

SEQ ID NO: 225 - protein, 553 aa, Panicum virgatum
MGSTNNWLGFASFSGAAAADDILPPLPPPRGDEAAAEPKLEDFLGLQEPAAGVAGRAPFAGSGGGASSIGLSMIKNWLRSQPAPGPAGADSMALAVVEEASTDEVRKVTDDRGAESVAAVVDAAQQRKAVAAVDTFGQRTSIYRGVTKHRWTGRYEAHLWDNSCRREGQTRKGRQVYLGGYDKEEKAARAYDLAALKYWGTTTTTNFPMSNYEKELEEMKHMSRQEYVASLRRKSSGFSRGASIYRGVTRHHQHGRWQARIGRVAGNKDLYLGTFSTQEEAAEAYDIAAIKFRGLNAVTNFDMSRYDVKSIMESSALPVGGTTKCLKDVHDQSDMGMNSSGADSASHMTATTKLLTDGIGSYGNENYGYSGWSPSAMMRIPLQFSNGQEHSRLWCKPEQDSAVVAAAHNLQHLQHFPSPGGTHDFFHPSHVQDVTGVADVSSPSVDPNSFLYNGVVGYHGSMGGGYAMPVATLVDSNHATSSYGVEEGTSELYSGQNLYYLSQASPGANTGKADAYEQQGVGYESWVPSVPVISQKDPNVTVCHGTPLFSVWK

SEQ ID NO: 226 - protein, 527 aa, Panicum hallii
MGSTNNWLGFASFSGAAAADDILPPLPPPRGDEADAEPKLEDFLGLQGPAAAVAGRPFAGSGGGASSIGLSMIKNWLRSQPAPGPTGADSMALAAVEAASTDGSGKVADGAVSDAAVVDAAQQRKAAAAVDTFGQRTSIYRGVTKHRWTGRYEAHLWDNSCRREGQTRKGRQVYLGGYDKEEKAARAYDLAALKYWGTTTTTNFPMSNYENELEEMKHMSRQEYVASLRRKSSGFSRGASIYRGVTRHHQHGRWQARIGRVAGNKDLYLGTFSTQEEAAEAYDIAAIKFRGLNAVTNFDMSRYNVKSIMESSALPVGGTTKRLKDVPDQSDMGMNSNVADSAGHMTATKLLTDGIGSYGHENYGYSGWSASAMTPIPLQFSNCHDHSRLWCKPEHDSAVVAAAHNLHHLQHFPDPGGTHNFFHPSPGQDVTGVTNVSSPSVDSNSFLYSGDVGYHGAMGGGYAMPVATLVDSNHAASGYGVEEGTSDLYSGRNLYYLSQASPGTNTGKADAYEQQGVGYESWVPSVR

SEQ ID NO: 227 - protein, 712 aa, Panicum hallii
MATVNNWLAFSLSPQDLPPSQTDSTLISAAATDEVSGDVCFNIPQDWGMRGSELSALVSEPKLEDFLGGINFSEQHHKANLNVIPSSSSTCYASSGASTGYHHQLYHHPSSALHFADSVMVASSAGVHDGGAMLSAAAANGGAGAAGANGGSIGLSMIKNWLRSQPAPPPQPRVAVAEGAQAAQGLSLSMNMAGTQGAGMPLLAGERGRAPESVSTSAQGGAVAARKEDSGGAGALVAVSTDTGGSGGASAETVARKTVDTFGQRTSIYRGVTRHRWTGRYEAHLWDNSCRREGQTRKGRQGGYDKEEKAARAYDLAALKYWGPTTTTNFPVSNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHAGVVSYDVGRIASQLGDGGALAAYGAHYHAAAAAAWPTIAFQPGATAGLYHPYAQPLPRGWCKKEQDHAVIAAAHSLQELNHLNLGAGAHDFFSAGQAAMHGLGSIDNSSLEHSTGSNSVVYNGVGDSNGGAVGGGYMMTMSAAAATTTAMVSHEQVHARAQGDHDEASKHAAQMGYESYLMNAEAAYGGGRMPSWTRPRLHRWRRRQAATTTWPALAMAARSSSVSGMTLNKLRTPTRHRGKKYADISNT

SEQ ID NO: 228 - DNA, 991 bp, Boechera stricta
tgaaaaatgttcctcaccctgatgtatgcagacattgattatggttattaaatatagttttttttttaatagtatgatgagaaaatatctgttcactgattttgaataaaagagtttgcgtttgggattaccattgcgtttatacaaggataagatccaccgaactggaatagcttcaaaccattaacagaacaaaaccaagttttgtctagaaacagagcatatagtaaacaaagagacaaaccattaacttgtcaaagaagatagttaatttatgcttttcagcagtctattttagaaccattatcatagtttgtaagtcaacagtgatacaaataacagagaaactagaaagctcgagaaagcacattcattatttttatatttgtatcggagattgcgaatatttgaccatatgttttagttcctacagagatatatgttttagataacggttatagactgtaatttgaccatatgttttagatatgttttgaacccggatagttacacaatgatggatagtataacatattatttagagtaaaacaaactggttacaaacgttaagctattaccctcggaaagaagttaaattataaactgaaaaaattatatatataataaatgtatttgtaacatgtggagatgtgatccttcacataacataaagttcaatgactgataaaatgcatagcataaagttcaatgagtaaaaaaagaagaagtatttgttcaccttctgtaacacgacgttaaaccttcgtctccaatacgaccgttactcattcaacaagccaatattaaatacccttccactatcaaatgctttaaagttccatgattaagtaaaaccatttaccgtaaatcattttacttactcttaaatcaccagtaagcttcgtatccatccaaaaccacgctataaaacacacaactctcacaactcaacccatcacatcacaacacaccagaactcaaaataagaata

(19) United States
(12) Patent Application Publication
(10) Pub. No.: US 2022/0356481 A1
(43) Pub. Date: Nov. 10, 2022

(54) WOX GENES

(71) Applicant: SYNGENTA CROP PROTECTION AG, Basel (CH)

(72) Inventors: Yuejin Sun, Research Triangle Park, NC (US); Qiudeng Que, Research Triangle Park, NC (US); Samson Prabhakar Nalapalli, Research Triangle Park, NC (US); Zhongying Zhong, Research Triangle Park, NC (US)

(73) Assignee: SYNGENTA CROP PROTECTION AG, Basel (CH)

(21) Appl. No.: 17/634,701

(22) PCT Filed: Aug. 10, 2020
(86) PCT No.: PCT/US2020/045573

§ 371 Date: Feb. 11, 2022

Related U.S. Application Data
(60) Provisional application No. 62/885,411, filed on Aug. 12, 2019.

(51) Int. Cl. C12N 15/82; C07K 14/415

(52) U.S. Cl. CPC C12N 15/8201; C07K 14/415; C12N 15/8233

(57) ABSTRACT

The invention provides methods for improving transformation efficiency of a plant. In some aspects, the methods according to the invention comprise the use of a WOX protein or WOX coding sequence, e.g., a nucleic acid encoding the amino acid sequence set forth in SEQ ID NO: 143 or a nucleic acid encoding a polypeptide comprising an amino acid sequence having at least 85% identity with the amino acid sequence set forth in SEQ ID NO: 143. Also provided are BABYBOOM coding sequences and methods of use thereof in improving transformation efficiency.

### RELATED APPLICATIONS

This application claims priority from provisional application 62/885,411 filed Aug. 12, 2019 and incorporated by reference in its entirety herein.

### FIELD OF THE INVENTION

The field of the invention is plant biotechnology and transformation, methods therefore, and nucleic acids and proteins useful in increasing transformation.

### SEQUENCE LISTING

This application is accompanied by a sequence listing entitled “WOXGENES_ST25.txt”, created Aug. 10, 2020, which is approximately 479 kilobytes in size. This sequence listing is incorporated herein by reference in its entirety. This sequence listing is submitted herewith via EFS-Web, and is in compliance with 37 C.F.R. § 1.824(a)(2)—(6) and (b).

### BACKGROUND

Current transformation technology provides an opportunity to engineer plants with desired traits. Major advances in plant transformation have occurred over the last few years. However, most transformation methods rely on the introduction of polynucleotides into embryonic tissues that are rapidly proliferating. Methods that allow for the transformation of more mature tissues would save considerable time and money. Accordingly, methods are needed in the art to increase transformation efficiencies of plants and allow for the transformation of more mature tissues.

When present, WUSCHEL proteins and their evolutionary relatives can improve transformation efficiency (U.S. Pat. No. 7,256,322, incorporated herein by reference in its entirety). The WUSCHEL protein, designated hereafter as WUS, plays a key role in the initiation and maintenance of the apical meristem, which contains a pool of pluripotent stem cells (Endrizzi et al., 1996, Plant Journal 10:967-979; Laux et al., 1996, Development 122:87-96; and Mayer et al., 1998, Cell 95:805-815). Arabidopsis plants mutant for the WUS gene contain stem cells that are misspecified and that appear to undergo differentiation. WUS encodes a novel homeodomain protein, which presumably functions as a transcriptional regulator (Mayer et al., 1998, Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand et al. (2000) Science 289:617-619; Schoof et al. (2000) Cell 100:635-644). Constitutive expression of WUS in Arabidopsis has been recently shown to lead to adventitious shoot proliferation from leaves (in planta) (Laux, T., Talk Presented at the XVI International Botanical Congress Meeting, Aug. 1-7, 1999, St. Louis, Mo.).

The WUSCHEL-related HOMEOBOX (WOX) gene family performs related functions during initiation and/or maintenance of various embryonic, meristematic, and organ initial cells (Haecker et al., 2004). Among the WUSCHEL-related HOMEOBOX (WOX) gene family proteins, WOX4 acts as a key regulator of TDIF signaling pathway (Hirakawa et al. 2010) and expressed preferentially in the procambium and cambium (Schrader et al., 2004; Ji et al., 2010 and Hirakawa et al. 2010; U.S. Pat. No. 10,125,371, incorporated herein by reference). For example, TDIF-TDR induces the transcription of master transcription factor WUSCHEL-related HOMEOBOX4 (WOX4) that promotes the maintenance of procambium/cambium stem cells in Arabidopsis and in Tomato. WUSCHEL-related HOMEOBOX4 (WOX4) polypeptide catalyzes the initiation of bast fiber in plant. However, there are not many characterization reports or existing technologies provided in the prior art relating to this polypeptide. U.S. Patent No. 2011/0283420 A1 (incorporated by reference) has disclosed WUSCHEL related homeobox 1-like (WOX1-like) polypeptide for enhanced yield-related traits in plants. In another E.P. Patent No. 1451301 B1 disclosed the use of WUSCHEL gene in promotion of somatic embryogenesis in plants. Recently, some WUSCHEL gene homologs were disclosed in U.S. Patent Application Publication No. 2010/0100981 A1 (incorporated herein by reference).

There is a great deal of interest in identifying the genes that encode proteins involved in development in plants, generally toward the objective of altering plant growth and architecture and improving transformation (Lowe et al., 2016, Gordon-Kamm et al 2019). SbWOX5 (SEQ ID NO: 143) represents one such protein. However, the SbWOX5 coding sequence (SEQ ID NO: 142) can also be used for the novel application of stimulating in vitro growth of plant tissue and improving transformation. In this manner, SbWOX5 can expand the range of tissues types targeted for transformation. Specifically, the SbWOX5 gene may be used to improve plant transformation frequencies and could result in genotype independent transformation of many important crops such as maize, soybean and sunflower. Furthermore, transformation into meristems would stimulate the formation of new apical initials reducing the chimeric nature of the transgenic events. Lastly, ectopic expression into non-meristematic cells would stimulate adventive meristem formation. This could lead to transformation of non-traditional tissues such as leaves, leaf bases, stem tissue, etc. Alternatively, transformation of a more traditional target such as callus or the scutellum of immature embryos could promote a “non-traditional” growth response, i.e. meristems in place of somatic embryos. In addition, SbWOX5 may also be used as a genetic marker for meristems.

### SUMMARY

One embodiment of the invention is a method for improving transformation efficiency of a plant, comprising transforming a plant with a nucleic acid encoding the amino acid sequence set forth in SEQ ID NO: 143 or a nucleic acid encoding a polypeptide comprising an amino acid sequence having an at least 85% identity (e.g., at least 85%, at least 90%, at least 95%, at least 98% or at least 99% identity) with the amino acid sequence set forth in SEQ ID NO: 143 and, optionally, having an effect that improves transformation efficiency of a plant. In another embodiment, the method comprises overexpressing an amino acid sequence having an at least 85% identity (e.g., at least 85%, at least 90%, at least 95%, at least 98% or at least 99% identity) with the amino acid sequence set forth in SEQ ID NO: 143, optionally wherein transformation efficiency of the plant is improved. In some embodiments, the nucleic acid encoding the amino acid sequence is a nucleic acid having a nucleic acid sequence of SEQ ID NO: 142 or a nucleic acid sequence having an at least 85% identity (e.g., at least 85%, at least 90%, at least 95%, at least 98% or at least 99% identity) with a nucleic acid sequence of SEQ ID NO: 142. In some embodiments of the method, the plant is a monocotyledon, and it may be, e.g., corn (i.e., maize), wheat, barley, rice, sorghum, and rye. In another aspect of the method, the plant is a dicotyledon, and it may be, e.g., soybean, sunflower, watermelon, or Arabidopsis. In another embodiment of the method the improvement of transformation efficiency of a plant comprises one or more of: (i) improvement of efficiency of callus formation of the plant; (ii) improvement of redifferentiation rate of the plant; and (iii) improvement of gene transfer efficiency.

Another embodiment of the invention is a nucleic acid construct comprising: (i) a nucleic acid encoding the amino acid sequence set forth in SEQ ID NO: 143 or a nucleic acid encoding a polypeptide comprising an amino acid sequence having at least 85% identity (e.g., at least 85%, at least 90%, at least 95%, at least 98% or at least 99% identity) with the amino acid sequence set forth in SEQ ID NO: 143 and, optionally, having an effect that improves transformation efficiency of a plant; and (ii) a promoter for producing a nucleic acid in the plant. In some embodiments, the promoter is a constitutive promoter, an inducible promoter, or a site-specific promoter. In another embodiment of the method comprises introducing into a plant a nucleic acid construct above, and further comprising a second nucleic acid to be expressed in the plant. In some embodiments, the transformation is transient. In another, it is stable. Another embodiment is a transformed plant obtained by the method of transformation.

Yet another embodiment of the invention is a nucleic acid construct comprising: (i) a nucleic acid encoding the amino acid sequence set forth in SEQ ID NO: 143 or a nucleic acid encoding a polypeptide comprising an amino acid sequence having at least 85% identity (e.g., at least 85%, at least 90%, at least 95%, at least 98% or at least 99% identity) with the amino acid sequence set forth in SEQ ID NO: 143; and (ii) a promoter for producing a nucleic acid in the plant; and (iii) optionally a desired nucleic acid to be produced in the plant; further optionally wherein the transformation efficiency is improved. In a further aspect, the nucleic acid construct further comprises a desired nucleic acid to be produced in the plant.

In another embodiment, the invention provides a method for improving transformation efficiency of a plant, comprising transforming a plant with (a) a nucleic acid encoding the amino acid sequence set forth in SEQ ID NO: 143 or a nucleic acid encoding a polypeptide comprising an amino acid sequence having an at least 85% identity (e.g., at least 85%, at least 90%, at least 95%, at least 98% or at least 99% identity) with the amino acid sequence set forth in SEQ ID NO: 143; and (b) a nucleic acid encoding a BABY BOOM amino acid sequence; optionally wherein the transformation efficiency of a plant is improved compared to a wildtype plant. In some embodiments, the nucleic acid encoding a BABY BOOM amino acid sequence is selected from the group consisting of SEQ ID NO: 179, SEQ ID NO: 180, and SEQ ID NO: 181.

In another embodiment, the invention provides a nucleic acid construct comprising: (a) a nucleic acid encoding the amino acid sequence set forth in SEQ ID NO: 143 or a nucleic acid encoding a polypeptide comprising an amino acid sequence having at least 85% identity (e.g., at least 85%, at least 90%, at least 95%, at least 98% or at least 99% identity) with the amino acid sequence set forth in SEQ ID NO: 143; (b) a nucleic acid encoding a BABY BOOM amino acid sequence; and (c) a promoter for producing a nucleic acid in the plant; optionally wherein the transformation efficiency is improved. In another embodiment, the nucleic acid construct further comprises a desired nucleic acid to be produced in the plant. In some embodiments, the nucleic acid construct comprises nucleic acid encoding a BABY BOOM amino acid sequence selected from the group consisting of SEQ ID NO: 179, SEQ ID NO: 180, and SEQ ID NO: 181.

The invention provides in some embodiments a nucleic acid construct comprising SEQ ID NO: 179, SEQ ID NO: 180, or SEQ ID NO: 181 operably linked to a heterologous regulatory sequence. Also provided is a method of increasing the transformation efficiency of a plant, comprising transforming a plant with a nucleic acid set forth in SEQ ID NO: 179 or a nucleic acid sequence having an at least 85% identity (e.g., at least 85%, at least 90%, at least 95%, at least 98% or at least 99% identity) with the sequence set forth in SEQ ID NO: 179, SEQ ID NO: 180, or SEQ ID NO: 181; optionally wherein the transformation efficiency of a plant is improved compared to a wildtype plant.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the promoter is an egg-cell preferred promoter.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the promoter is SEQ ID NO. 288.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the plant is a monocotyledon.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the monocotyledon is corn.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the plant comprises the matrilineal haploid induction locus.

In another embodiment, the invention provides a haploid plant obtained by the method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant.

In another embodiment, the invention provides a recombinant DNA molecule comprising a DNA sequence selected from the group consisting of: a) a sequence with at least 85 percent sequence identity to SEQ ID NO:288; b) a fragment of SEQ ID NO:288, wherein the fragment has gene-regulatory activity; wherein said DNA sequence is operably linked to a heterologous transcribable DNA molecule.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant comprises the matrilineal haploid induction locus.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant comprises modifications to alter meiosis to mitosis.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant comprises modifications to alter meiosis to mitosis, wherein the plant comprises knockouts of the meiotic genes REC8, PAIR1, and OSD1.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the promoter is an egg-cell preferred promoter.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the promoter is an egg-cell preferred promoter, wherein the promoter is SEQ ID NO. 288.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant is a monocotyledon.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant is a monocotyledon, wherein the monocotyledon is corn.

In another embodiment, the invention provides a plant produced by the method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization.

### BRIEF DESCRIPTION OF THE SEQUENCES IN THE SEQUENCE LISTING

SEQ ID NO: 1 is the AT3G18010.1_ARATH WOX 1 coding sequence from Arabidopsis thaliana.

SEQ ID NO: 2 is the AT3G18010.1_ARATH WOX 1 protein from Arabidopsis thaliana.

SEQ ID NO: 3 is the Bra001694 coding sequence from Brassica rapa.

SEQ ID NO: 4 is the Bra001694 protein from Brassica rapa.

SEQ ID NO: 5 is the bra022267 coding sequence from Brassica rapa.

SEQ ID NO: 6 is the Bra022267 protein from Brassica rapa.

SEQ ID NO: 7 is the Medtr3g088485.1 coding sequence from Medicago truncatula.

SEQ ID NO: 8 is the Medtr3g088485.1 protein from Medicago truncatula.

SEQ ID NO: 9 is the Medtr4g084550.1 coding sequence from Medicago truncatula.

SEQ ID NO: 10 is the Medtr4g084550.1 protein from Medicago truncatula.

SEQ ID NO: 11 is the Medtr8g095580.1 coding sequence from Medicago truncatula.

SEQ ID NO: 12 is the Medtr8g095580.1 protein from Medicago truncatula.

SEQ ID NO: 13 is the Medtr8g107210.1 coding sequence from Medicago truncatula.

SEQ ID NO: 14 is the Medtr8g107210.1 protein from Medicago truncatula.

SEQ ID NO: 15 is the Phvul.002G095800.1.p coding sequence from Phaseolus vulgaris.

SEQ ID NO: 16 is the Phvul.002G095800.1.p protein from Phaseolus vulgaris.

SEQ ID NO: 17 is the Phvul.002G329500.1.p coding sequence from Phaseolus vulgaris.

SEQ ID NO: 18 is the Phvul.002G329500.1.p protein from Phaseolus vulgaris.

SEQ ID NO: 19 is the Phvul.L002200.1.p coding sequence from Phaseolus vulgaris.

SEQ ID NO: 20 is the Phvul.L002200.1.p protein from Phaseolus vulgaris.

SEQ ID NO: 21 is the ZmWOX (DP Seq14) protein from Zea mays.

SEQ ID NO: 22 is the AT5G59340.1_ARATH WOX2 coding sequence from Arabidopsis thaliana.

SEQ ID NO: 23 is the AT5G59340.1_ARATH WOX2 protein from Arabidopsis thaliana.

SEQ ID NO: 24 is the Bra002576 coding sequence from Brassica rapa.

SEQ ID NO: 25 is the Bra002576 protein from Brassica rapa.

SEQ ID NO: 26 is the Bradi2g54590.1.p coding sequence from Brachypodium distachyon (BdWOX2).

SEQ ID NO: 27 is the Bradi2g54590.1.p protein from Brachypodium distachyon (BdWOX2).

SEQ ID NO: 28 is the GRMZM2G108933_P01 coding sequence from Zea mays.

SEQ ID NO: 29 is the GRMZM2G108933_P01 protein from Zea mays.

SEQ ID NO: 30 is the GRMZM2G339751_P01 coding sequence from Zea mays.

SEQ ID NO: 31 is the GRMZM2G339751_P01 protein from Zea mays.

SEQ ID NO: 32 is the LOC_Os01g62310.1 coding sequence from Oryza sativa.

SEQ ID NO: 33 is the LOC_Os01g62310.1 protein from Oryza sativa.

SEQ ID NO: 34 is the Medtr4g063735.1 coding sequence from Medicago truncatula.

SEQ ID NO: 35 is the Medtr4g063735.1 protein from Medicago truncatula.

SEQ ID NO: 36 is the Phvul.005G142900.1.p coding sequence from Phaseolus vulgaris.

SEQ ID NO: 37 is the Phvul.005G142900.1.p protein from Phaseolus vulgaris.

SEQ ID NO: 38 is the Phvul.011G064900.1.p coding sequence from Phaseolus vulgaris.

SEQ ID NO: 39 is the Phvul.011G064900.1.p protein from Phaseolus vulgaris.

SEQ ID NO: 40 is the Sobic.003G350900.1.p coding sequence from Sorghum bicolor.

SEQ ID NO: 41 is the Sobic.003G350900.1.p protein from Sorghum bicolor.

SEQ ID NO: 42 is the Traes_3B_669466D5C.1 coding sequence from Triticum aestivum.

SEQ ID NO: 43 is the Traes_3B_669466D5C.1 protein from Triticum aestivum.

SEQ ID NO: 44 is the Traes_5DS_903A67B97.2 coding sequence from Triticum aestivum.

SEQ ID NO: 45 is the Traes_5DS_903A67B97.2 protein from Triticum aestivum.

SEQ ID NO: 46 is the AT2G28610.1_ARATH WOX3 coding sequence from Arabidopsis thaliana.

SEQ ID NO: 47 is the AT2G28610.1_ARATH WOX3 protein from Arabidopsis thaliana.

SEQ ID NO: 48 is the Bra000484 coding sequence from Brassica rapa.

SEQ ID NO: 49 is the Bra000484 protein from Brassica rapa.

SEQ ID NO: 50 is the Bra035688 coding sequence from Brassica rapa.

SEQ ID NO: 51 is the Bra035688 protein from Brassica rapa.

SEQ ID NO: 52 is the Bradi2g37650.1.p coding sequence from Brachypodium distachyon.

SEQ ID NO: 53 is the Bradi2g37650.1.p protein from Brachypodium distachyon.

SEQ ID NO: 54 is the Bradi4g45325.1.p coding sequence from Brachypodium distachyon (BdWOX3).

SEQ ID NO: 55 is the Bradi4g45325.1.p protein from Brachypodium distachyon (BdWOX3).

SEQ ID NO: 56 is the GRMZM2G069028_P01 coding sequence from Zea mays.

SEQ ID NO: 57 is the GRMZM2G069028_P01 protein from Zea mays.

SEQ ID NO: 58 is the GRMZM2G122537_P02 coding sequence from Zea mays.

SEQ ID NO: 59 is the GRMZM2G122537_P02 protein from Zea mays.

SEQ ID NO: 60 is the GRMZM2G140083_P01 coding sequence from Zea mays.

SEQ ID NO: 61 is the GRMZM2G140083_P01 protein from Zea mays.

SEQ ID NO: 62 is the LOC_Os05g02730.1 coding sequence from Oryza sativa.

SEQ ID NO: 63 is the LOC_Os05g02730.1 protein from Oryza sativa.

SEQ ID NO: 64 is the LOC_Os11g01130.2 coding sequence from Oryza sativa.

SEQ ID NO: 65 is the LOC_Os11g01130.2 protein from Oryza sativa.

SEQ ID NO: 66 is the LOC_Os12g01120.1 coding sequence from Oryza sativa.

SEQ ID NO: 67 is the LOC_Os12g01120.1 protein from Oryza sativa.

SEQ ID NO: 68 is the Medtr7g060630.1 coding sequence from Medicago truncatula.

SEQ ID NO: 69 is the Medtr7g060630.1 protein from Medicago truncatula.

SEQ ID NO: 70 is the Phvul.008G100800.1.p coding sequence from Phaseolus vulgaris.

SEQ ID NO: 71 is the Phvul.008G100800.1.p protein from Phaseolus vulgaris.

SEQ ID NO: 72 is the Sobic.005G042200.1.p coding sequence from Sorghum bicolor.

SEQ ID NO: 73 is the Sobic.005G042200.1.p protein from Sorghum bicolor.

SEQ ID NO: 74 is the Sobic.009G023900.1.p coding sequence from Sorghum bicolor.

SEQ ID NO: 75 is the Sobic.009G023900.1.p protein from Sorghum bicolor.

SEQ ID NO: 76 is the Traes_1 AS_3 CA8D36FB.1 coding sequence from Triticum aestivum.

SEQ ID NO: 77 is the Traes_1 AS_3 CA8D36FB.1 protein from Triticum aestivum.

SEQ ID NO: 78 is the Traes_1 BS_C908081B8.1 coding sequence from Triticum aestivum.

SEQ ID NO: 79 is the Traes_1 BS_C908081B8.1 protein from Triticum aestivum.

SEQ ID NO: 80 is the Traes_1DS_E50CDDF05.1 coding sequence from Triticum aestivum.

SEQ ID NO: 81 is the Traes_1DS_E50CDDF05.1 protein from Triticum aestivum.

SEQ ID NO: 82 is the Traes_5 BL_2E6FA4A97.1 coding sequence from Triticum aestivum.

SEQ ID NO: 83 is the Traes_5 BL_2E6FA4A97.1 protein from Triticum aestivum.

SEQ ID NO: 84 is the Traes_5 DL_193218298.1 coding sequence from Triticum aestivum.

SEQ ID NO: 85 is the Traes_5 DL_193218298.1 protein from Triticum aestivum.

SEQ ID NO: 86 is the AT1G46480.1_ARATH WOX4 coding sequence from Arabidopsis thaliana.

SEQ ID NO: 87 is the AT1G46480.1_ARATH WOX4 protein from Arabidopsis thaliana.

SEQ ID NO: 88 is the Bra014055 coding sequence from Brassica rapa.

SEQ ID NO: 89 is the Bra014055 protein from Brassica rapa.

SEQ ID NO: 90 is the Bra032212 coding sequence from Brassica rapa.

SEQ ID NO: 91 is the Bra032212 protein from Brassica rapa.

SEQ ID NO: 92 is the Bradi5g24080.1.p coding sequence from Brachypodium distachyon (BdWOX4).

SEQ ID NO: 93 is the Bradi5g24080.1.p protein from Brachypodium distachyon (BdWOX4).

SEQ ID NO: 94 is the LOC_Os04g55590.1 coding sequence from Oryza sativa.

SEQ ID NO: 95 is the LOC_Os04g55590.1 protein from Oryza sativa.

SEQ ID NO: 96 is the Medtr1g019130.1 coding sequence from Medicago truncatula.

SEQ ID NO: 97 is the Medtr1g019130.1 protein from Medicago truncatula.

SEQ ID NO: 98 is the Medtr1g019130.2 coding sequence from Medicago truncatula.

SEQ ID NO: 99 is the Medtr1g019130.2 protein from Medicago truncatula.

SEQ ID NO: 100 is the Phvul.001G023600.1.p coding sequence from Phaseolus vulgaris.

SEQ ID NO: 101 is the Phvul.001G023600.1.p protein from Phaseolus vulgaris.

SEQ ID NO: 102 is the Phvul.008G098800.1.p coding sequence from Phaseolus vulgaris.

SEQ ID NO: 103 is the Phvul.008G098800.1.p protein from Phaseolus vulgaris.

SEQ ID NO: 104 is the Sobic.006G241000.1.p coding sequence from Sorghum bicolor.

SEQ ID NO: 105 is the Sobic.006G241000.1.p protein from Sorghum bicolor.

SEQ ID NO: 106 is the Traes_2AL_BF4D53AA5.1 coding sequence from Triticum aestivum.

SEQ ID NO: 107 is the Traes_2AL_BF4D53AA5.1 protein from Triticum aestivum.

SEQ ID NO: 108 is the Traes_2BL_7AED4E232.1 coding sequence from Triticum aestivum.

SEQ ID NO: 109 is the Traes_2BL_7AED4E232.1 protein from Triticum aestivum.

SEQ ID NO: 110 is the Traes_2 DL_467797574.2 coding sequence from Triticum aestivum.

SEQ ID NO: 111 is the Traes_2 DL_467797574.2 protein from Triticum aestivum.

SEQ ID NO: 112 is the AT3G11260.1_ARATH WOX5 coding sequence from Arabidopsis thaliana.

SEQ ID NO: 113 is the AT3G11260.1_ARATH WOX5 protein from Arabidopsis thaliana.

SEQ ID NO: 114 is the AT5G05770.1_ARATH WOX7 coding sequence from Arabidopsis thaliana.

SEQ ID NO: 115 is the AT5G05770.1_ARATH WOX7 protein from Arabidopsis thaliana.

SEQ ID NO: 116 is the Bra009132 coding sequence from Brassica rapa.

SEQ ID NO: 117 is the Bra009132 protein from Brassica rapa.

SEQ ID NO: 118 is the Bra028749 coding sequence from Brassica rapa.

SEQ ID NO: 119 is the Bra028749 protein from Brassica rapa.

SEQ ID NO: 120 is the Bra034855 coding sequence from Brassica rapa.

SEQ ID NO: 121 is the Bra034855 protein from Brassica rapa.

SEQ ID NO: 122 is the Bradi2g55270.1.p coding sequence from Brachypodium distachyon (BdWOX5).

SEQ ID NO: 123 is the Bradi2g55270.1.p protein from Brachypodium distachyon (BdWOX5).

SEQ ID NO: 124 is the GRMZM2G116063_P01 coding sequence from Zea mays.

SEQ ID NO: 125 is the GRMZM2G116063_P01 protein from Zea mays.

SEQ ID NO: 126 is the GRMZM2G478396_P01 coding sequence from Zea mays.

SEQ ID NO: 127 is the GRMZM2G478396_P01 protein from Zea mays.

SEQ ID NO: 128 is the LOC_Os01g63510.1 coding sequence from Oryza sativa.

SEQ ID NO: 129 is the LOC_Os01g63510.1 protein from Oryza sativa.

SEQ ID NO: 130 is the Medtr5g081990.1 coding sequence from Medicago truncatula.

SEQ ID NO: 131 is the Medtr5g081990.1 protein from Medicago truncatula.

SEQ ID NO: 132 is the Phvul.001G241000.1.p coding sequence from Phaseolus vulgaris.

SEQ ID NO: 133 is the Phvul.001G241000.1.p protein from Phaseolus vulgaris.

SEQ ID NO: 134 is the Phvul.008G226100.1.p coding sequence from Phaseolus vulgaris.

SEQ ID NO: 135 is the Phvul.008G226100.1.p protein from Phaseolus vulgaris.

SEQ ID NO: 136 is the Sobic.003G360200.1.p coding sequence from Sorghum bicolor.

SEQ ID NO: 137 is the Sobic.003G360200.1.p protein from Sorghum bicolor.

SEQ ID NO: 138 is the Traes_3B_7E3E06FD6.2 coding sequence from Triticum aestivum.

SEQ ID NO: 139 is the Traes_3B_7E3E06FD6.2 protein from Triticum aestivum.

SEQ ID NO: 140 is the OsWOX5 coding sequence from Oryza sativa.

SEQ ID NO: 141 is the OsWOX5 protein from Oryza sativa.

SEQ ID NO: 142 is the SbWOX5 coding sequence from Sorghum bicolor.

SEQ ID NO: 143 is the SbWOX5 protein from Sorghum bicolor.

SEQ ID NO: 144 is the TaWOX5 coding sequence from Triticum aestivum.

SEQ ID NO: 145 is the TaWOX5 protein from Triticum aestivum.

SEQ ID NO: 146 is the ZmWOX (DP Seq4) protein from Zea mays.

SEQ ID NO: 147 is the AT2G01500.1_ARATH WOX6 coding sequence from Arabidopsis thaliana.

SEQ ID NO: 148 is the AT2G01500.1_ARATH WOX6 protein from Arabidopsis thaliana.

SEQ ID NO: 149 is the Bra017448 coding sequence from Brassica rapa.

SEQ ID NO: 150 is the Bra017448 protein from Brassica rapa.

SEQ ID NO: 151 is the Bra026791 coding sequence from Brassica rapa.

SEQ ID NO: 152 is the Bra026791 protein from Brassica rapa.

SEQ ID NO: 153 is the AT2G17950.1 WUS coding sequence from Arabidopsis thaliana.

SEQ ID NO: 154 is the AT2G17950.1 WUS protein from Arabidopsis thaliana.

SEQ ID NO: 155 is the Bra024485 coding sequence from Brassica rapa.

SEQ ID NO: 156 is the Bra024485 protein from Brassica rapa.

SEQ ID NO: 157 is the Bra039894 coding sequence from Brassica rapa.

SEQ ID NO: 158 is the Bra039894 protein from Brassica rapa.

SEQ ID NO: 159 is the Bra037245 coding sequence from Brassica rapa.

SEQ ID NO: 160 is the Bra037245 protein from Brassica rapa.

SEQ ID NO: 161 is the BRADI5G25113.1.P coding sequence from Brachypodium distachyon (BdWUS).

SEQ ID NO: 162 is the BRADI5G25113.1.P protein from Brachypodium distachyon (BdWUS).

SEQ ID NO: 163 is the GRMZM2G047448_P01 protein from Zea mays.

SEQ ID NO: 164 is the LOC_Os04g56780.1 coding sequence from Oryza sativa.

SEQ ID NO: 165 is the LOC_Os04g56780.1 protein from Oryza sativa.

SEQ ID NO: 166 is the Medtr5g021930.1 coding sequence from Medicago truncatula.

SEQ ID NO: 167 is the Medtr5g021930.1 protein from Medicago truncatula.

SEQ ID NO: 168 is the Phvul.002G109400.1.p coding sequence from Phaseolus vulgaris.

SEQ ID NO: 169 is the Phvul.002G109400.1.p protein from Phaseolus vulgaris.

SEQ ID NO: 170 is the Sobic.006G254900.1.p coding sequence from Sorghum bicolor.

SEQ ID NO: 171 is the Sobic.006G254900.1.p protein from Sorghum bicolor.

SEQ ID NO: 172 is the GRMZM2G028622_T01 coding sequence from Zea mays.

SEQ ID NO: 173 is the GRMZM2G028622_T01 protein from Zea mays.

SEQ ID NO: 174 is the ZmWUS2 (ABW43772) protein from Zea mays.

SEQ ID NO: 175 is the ZmWOX (DP Seq6) protein from Zea mays.

SEQ ID NO: 176 is the ZmWOX (DP Seq8) protein from Zea mays.

SEQ ID NO: 177 is the PMI coding sequence from E. coli.

SEQ ID NO: 178 is the synthetic CFP gene.

SEQ ID NO: 179 is the BABY BOOM1 coding sequence from foxtail millet (Setaria italica).

SEQ ID NO: 180 is the BABY BOOM1 coding sequence from Brachypodium distachyon.

SEQ ID NO: 181 is the BABY BOOM1 coding sequence from Brassica napus, codon optimized for maize expression.

SEQ ID NOs: 182-227 are described in Table 1.

SEQ ID NO: 228 is a root preferred promoter from Boechera stricta.

### BRIEF DESCRIPTION OF THE DRAWINGS

FIG. 1 shows the molecular phylogenetic analysis by the maximum likelihood method. The evolutionary history was inferred by using the Maximum Likelihood method based on the JTT matrix-based model. See Jones D. T., Taylor W. R., and Thornton J. M. (1992). The rapid generation of mutation data matrices from protein sequences. See Computer Applications in the Biosciences 8: 275-282. The tree with the highest log likelihood (−1123.5348) is shown. Initial tree for the heuristic search was obtained automatically by applying Neighbor-Join and BioNJ algorithms to a matrix of pairwise distances estimated using a JTT model, and then selecting the topology with superior log likelihood value. The tree is drawn to scale, with branch lengths measured in the number of substitutions per site. The analysis involved 91 amino acid sequences. All positions containing gaps and missing data were eliminated. There were a total of 30 positions in the final dataset. Evolutionary analyses were conducted in MEGA7. See Kumar S., Stecher G., and Tamura K. (2015) MEGA7: Molecular Evolutionary Genetics Analysis version 7.0 for bigger datasets, Molecular Biology and Evolution (submitted).

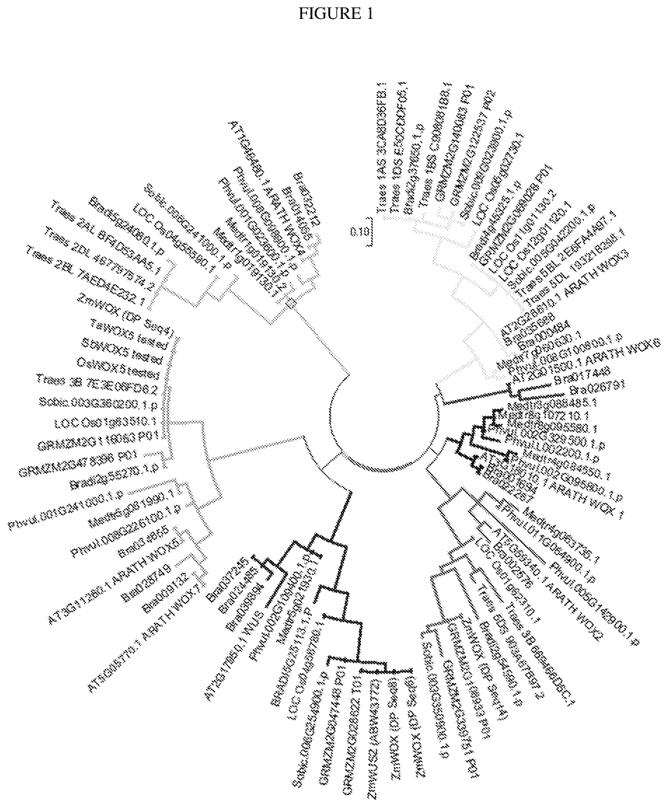

### DEFINITIONS

This invention is not limited to the particular methodology, protocols, cell lines, plant species or genera, constructs, and reagents described herein as such. The terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a plant” is a reference to one or more plants and includes equivalents thereof known to those skilled in the art, and so forth. As used herein, the word “or” means any one member of a particular list and also includes any combination of members of that list (i.e., includes also “and”).

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this invention (e.g., in combinations with temperature or molecular weight values) the exact value (i.e., without “about”) is preferred.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASB A, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

The term “specific DNA sequence” indicates a polynucleotide sequence having a nucleotide sequence homology of more than 80%, preferably more than 85%, more preferably more than 90%, even more preferably more than 95%, still more preferably more than 97%, most preferably more than 99% with another named sequence.

“cDNA” refers to a single-stranded or a double-stranded DNA that is complementary to and derived from mRNA.

The term “chimeric construct”, “chimeric gene”, “chimeric polynucleotide” or chimeric nucleic acid” (and similar terms) as used herein refers to a construct or molecule comprising two or more polynucleotides of different origin assembled into a single nucleic acid molecule. The term “chimeric construct”, “chimeric gene”, “chimeric polynucleotide” or “chimeric nucleic acid” refers to any construct or molecule that contains (1) polynucleotides (e.g., DNA), including regulatory and coding polynucleotides that are not found together in nature (i.e., at least one of polynucleotides is heterologous with respect to at least one of its other polynucleotides), or (2) polynucleotides encoding parts of proteins not naturally adjoined, or (3) parts of promoters that are not naturally adjoined. Further, a chimeric construct, chimeric gene, chimeric polynucleotide or chimeric nucleic acid may comprise regulatory polynucleotides and coding polynucleotides that are derived from different sources, or comprise regulatory polynucleotides and coding polynucleotides derived from the same source, but arranged in a manner different from that found in nature. In a preferred aspect of the present invention the chimeric construct, chimeric gene, chimeric polynucleotide or chimeric nucleic acid comprises an expression cassette comprising a polynucleotides of the present invention under the control of regulatory polynucleotides, particularly under the control of regulatory polynucleotides functional in plants.

The term “chromosome” is used herein as recognized in the art as meaning the self-replicating genetic structure in the cellular nucleus containing the cellular DNA and bearing the linear array of genes.

A “coding polynucleotide” is a polynucleotide that is transcribed into RNA, such as mRNA, rRNA, tRNA, snRNA, sense RNA or antisense RNA. Preferably the RNA is then translated in an organism to produce a protein. It may constitute an “uninterrupted coding polynucleotide”, i.e., lacking an intron, such as in a cDNA, or it may include one or more introns bounded by appropriate splice junctions. An “intron” is a poly(ribo)nucleotide which is contained in the primary transcript but which is removed through cleavage and religation of the RNA within the cell to create the mature mRNA that can be translated into a protein.

“dsRNA” or “double-stranded RNA” is RNA with two substantially complementary strands, which directs the sequence-specific degradation of mRNA through a process known as RNA interference (RNAi). dsRNA is cut into siRNAs interfering with the expression of a specific gene.

As used herein, explant refers to an immature embryo isolated from the seed or kernel. For maize elite lines, explants are obtained approximately 9 days after pollination (“DAP”) and 8 DAP for sweet corn lines.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

“Expression cassette” as used herein means a nucleic acid molecule capable of directing expression of a particular polynucleotide or polynucleotides in an appropriate host cell, comprising a promoter operably linked to the polynucleotide or polynucleotides of interest which is/are operably linked to termination signals. It also typically comprises polynucleotides required for proper translation of the polynucleotide or polynucleotides of interest. The expression cassette may also comprise polynucleotides not necessary in the direct expression of a polynucleotide of interest but which are present due to convenient restriction sites for removal of the cassette from an expression vector. The expression cassette comprising the polynucleotide(s) of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e. the particular polynucleotide of the expression cassette does not occur naturally in the host cell and must have been introduced into the host cell or an ancestor of the host cell by a transformation process known in the art. The expression of the polynucleotide(s) in the expression cassette is generally under the control of a promoter. In the case of a multicellular organism, such as a plant, the promoter can also be specific or preferential to a particular tissue, or organ, or stage of development. An expression cassette, or fragment thereof, can also be referred to as “inserted polynucleotide” or “insertion polynucleotide” when transformed into a plant.

A “gene” is defined herein as a hereditary unit consisting of a polynucleotide that occupies a specific location on a chromosome and that contains the genetic instruction for a particular characteristic or trait in an organism, or such hereditary unit from a group of heterologous organisms depending on context.

“Genetic engineering,” “transformation,” and “genetic modification” are all used herein as synonyms for the transfer of isolated and cloned genes into the DNA, usually the chromosomal DNA or genome, of another organism.

The term “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait. In some embodiments, a genotype is expressed in terms of a haplotype (defined herein below).

The term “heterologous” when used in reference to a gene or nucleic acid refers to a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide.

The terms “homology”, “sequence similarity” or “sequence identity” of nucleotide or amino acid sequences mean a degree of identity or similarity of two or more sequences and may be determined conventionally by using known software or computer programs such as the Best-Fit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2:482-489 (1981), to find the best segment of identity or similarity between two sequences. Sequence comparison between two or more polynucleotides or polypeptides is generally performed by comparing portions of the two sequences over a comparison window to identify and compare local regions of sequence similarity. The comparison window is generally from about 20 to 200 contiguous nucleotides. Gap performs global alignments: all of one sequence with all of another similar sequence using the method of Needleman and Wunsch, J. Mol. Biol. 48:443-453 (1970). When using a sequence alignment program such as BestFit to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity, or homology between two different amino acid sequences, the default settings may be used, or an appropriate scoring matrix, such as blosum45 or blosum80, may be selected to optimize identity, similarity, or homology scores.

“Homologous recombination” is the exchange (“crossing over”) of DNA fragments between two DNA molecules or chromatids of paired chromosomes in a region of identical polynucleotides. A “recombination event” is herein understood to mean a meiotic crossing-over.

The term “heterozygous” means a genetic condition existing when different alleles reside at corresponding loci on homologous chromosomes.

The term “homozygous” means a genetic condition existing when identical alleles reside at corresponding loci on homologous chromosomes.

The term “hybrid” in the context of nucleic acids refers to a double-stranded nucleic acid molecule, or duplex, formed by hydrogen bonding between complementary nucleotide bases. The terms “hybridize” or “anneal” refer to the process by which single strands of polynucleotides form double-helical segments through hydrogen bonding between complementary bases.

The term “isolated,” when used in the context of the nucleic acid molecules or polynucleotides of the present invention, refers to a polynucleotide that is identified within and isolated/separated from its chromosomal polynucleotide context within the respective source organism. An isolated nucleic acid or polynucleotide is not a nucleic acid as it occurs in its natural context, if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In a preferred embodiment, the nucleic acid molecules of the present invention are understood to be isolated.

The term “linkage,” and grammatical variants thereof, refers to the tendency of alleles at different loci on the same chromosome to segregate together more often than would be expected by chance if their transmission were independent, in some embodiments as a consequence of their physical proximity.

The phrase “linkage disequilibrium” (also called “allelic association”) refers to a phenomenon wherein particular alleles at two or more loci tend to remain together in linkage groups when segregating from parents to offspring with a greater frequency than expected from their individual frequencies in a given population. For example, a genetic marker allele and a QTL allele can show linkage disequilibrium when they occur together with frequencies greater than those predicted from the individual allele frequencies. Linkage disequilibrium can occur for several reasons including, but not limited to the alleles being in close proximity on a chromosome.

The term “linkage group” refers to all of the genes or genetic traits that are located on the same chromosome. Within the linkage group, those loci that are close enough together will exhibit linkage in genetic crosses. Since the probability of crossover increases with the physical distance between genes on a chromosome, genes whose locations are far removed from each other within a linkage group may not exhibit any detectable linkage in direct genetic tests. The term “linkage group” is mostly used to refer to genetic loci that exhibit linked behavior in genetic systems where chromosomal assignments have not yet been made. Thus, in the present context, the term “linkage group” is synonymous to (the physical entity of) chromosome.

The term “locus” refers to a position (e.g., of a gene, a genetic marker, or the like) on a chromosome of a given species.

The terms “messenger RNA” or “mRNA” refer to RNA that does not comprise introns and that can be translated into a protein by the cell.

The terms “molecular marker” or “genetic marker” refer to an indicator that is used in methods for visualizing differences in characteristics of polynucleotides. It refers to a feature of an individual's genome (e.g., a polynucleotide that is present in an individual's genome) that is associated with one or more loci of interest. In some embodiments, a genetic marker is polymorphic in a population of interest or the locus occupied by the polymorphism, depending on the context. Genetic markers include, for example, single nucleotide polymorphisms (SNPs), indels (i.e., insertions/deletions), simple sequence repeats (also named microsatellite markers; SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Additional markers include insertion mutations, sequence-characterized amplified regions (SCARs), or isozyme markers or combinations of the markers described herein which defines a specific genetic and chromosomal location. Genetic markers can, for example, be used to locate genetic loci containing alleles that contribute to variability in expression of phenotypic traits on a chromosome. The phrase “genetic marker” can also refer to the sequence of a polynucleotide complementary to a genomic polynucleotide, such as a sequence of a nucleic acid used as a probe. A genetic marker can be physically located in a position on a chromosome that is within or outside of the genetic locus with which it is associated (i.e., is intragenic or extragenic, respectively). Stated another way, whereas genetic markers are typically employed when the location on a chromosome of the gene that corresponds to the locus of interest has not been identified and there is a non-zero rate of recombination between the genetic marker and the locus of interest, the presently disclosed subject matter can also employ genetic markers that are physically within the boundaries of a genetic locus (e.g., inside a genomic polynucleotide that corresponds to a gene such as, but not limited to a polymorphism within an intron or an exon of a gene).

The term “microsatellite or SSRs (simple sequence repeats) marker” is understood within the scope of the invention to refer to a type of genetic marker that comprises numerous repeats of short sequences of DNA bases, which are found at loci throughout the plant's DNA and have a likelihood of being highly polymorphic.

The phrase “nucleic acid” or “polynucleotide” refers to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

“Operably linked” refers to the association of polynucleotides on a single nucleic acid fragment so that the function of one affects the function of the other. For example, a promoter is operably linked with a coding polynucleotide or functional RNA when it is capable of affecting the expression of that coding polynucleotide or functional RNA (i.e., that the coding polynucleotide or functional RNA is under the transcriptional control of the promoter). Coding polynucleotide in sense or antisense orientation can be operably linked to regulatory polynucleotides.

“PCR (polymerase chain reaction)” is understood within the scope of the invention to refer to a method of producing relatively large amounts of specific regions of DNA, thereby making possible various analyses that are based on those regions.

“Polymorphism” is understood within the scope of the invention to refer to the presence in a population of two or more different forms of a gene, genetic marker, or inherited trait.

The term “probe” refers to a single-stranded oligonucleotide that will form a hydrogen-bonded duplex with a substantially complementary oligonucleotide in a target nucleic acid analyte or its cDNA derivative.

The term “primer”, as used herein, refers to an oligonucleotide which is capable of annealing to the amplification target allowing a DNA polymerase to attach, thereby serving as a point of initiation of DNA synthesis when placed under conditions in which synthesis of primer extension product is induced, e.g., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer is generally sufficiently long to prime the synthesis of extension products in the presence of the agent for polymerization. The exact lengths of the primers will depend on many factors, including temperature and composition (A/T and G/C content) of primer. A pair of bi-directional primers consists of one forward and one reverse primer as commonly used in the art of DNA amplification such as in PCR amplification. It will be understood that “primer,” as used herein, may refer to more than one primer, particularly in the case where there is some ambiguity in the information regarding the terminal sequence(s) of the target region to be amplified. Hence, a “primer” includes a collection of primer oligonucleotides containing sequences representing the possible variations in the sequence or includes nucleotides which allow a typical base pairing. The oligonucleotide primers may be prepared by any suitable method. Methods for preparing oligonucleotides of specific sequence are known in the art, and include, for example, cloning and restriction of appropriate sequences, and direct chemical synthesis. Chemical synthesis methods may include, for example, the phospho di- or tri-ester method, the diethylphosphoramidate method and the solid support method disclosed in, for example, U.S. Pat. No. 4,458,066. The primers may be labeled, if desired, by incorporating means detectable by, for instance, spectroscopic, fluorescence, photochemical, biochemical, immunochemical, or chemical means. Template-dependent extension of the oligonucleotide primer(s) is catalyzed by a polymerizing agent in the presence of adequate amounts of the four deoxyribonucleotide triphosphates (dATP, dGTP, dCTP and dTTP, i.e. dNTPs) or analogues, in a reaction medium which is comprised of the appropriate salts, metal cations, and pH buffering system. Suitable polymerizing agents are enzymes known to catalyze primer- and template-dependent DNA synthesis. Known DNA polymerases include, for example, E. coli DNA polymerase I or its Klenow fragment, T4 DNA polymerase, and Taq DNA polymerase. The reaction conditions for catalyzing DNA synthesis with these DNA polymerases are known in the art. The products of the synthesis are duplex molecules consisting of the template strands and the primer extension strands, which include the target sequence. These products, in turn, serve as template for another round of replication. In the second round of replication, the primer extension strand of the first cycle is annealed with its complementary primer; synthesis yields a “short” product which is bound on both the 5′- and the 3′-ends by primer sequences or their complements. Repeated cycles of denaturation, primer annealing, and extension result in the exponential accumulation of the target region defined by the primers. Sufficient cycles are run to achieve the desired amount of polynucleotide containing the target region of nucleic acid. The desired amount may vary, and is determined by the function which the product polynucleotide is to serve. The PCR method is well described in handbooks and known to the skilled person. After amplification by PCR, the target polynucleotides may be detected by hybridization with a probe polynucleotide which forms a stable hybrid with that of the target sequence under low, moderate, or even highly stringent hybridization and wash conditions. If it is expected that the probes will be essentially completely complementary (i.e., about 99% or greater) to the target sequence, highly stringent conditions may be used. If some mismatching is expected, for example if variant strains are expected with the result that the probe will not be completely complementary, the stringency of hybridization may be lessened. However, conditions are typically chosen which rule out nonspecific/adventitious binding. Conditions, which affect hybridization, and which select against nonspecific binding are known in the art, and are described in, for example, Sambrook and Russell, 2001. Generally, lower salt concentration and higher temperature increase the stringency of hybridization conditions. “PCR primer” is preferably understood within the scope of the present invention to refer to relatively short fragments of single-stranded DNA used in the PCR amplification of specific regions of DNA.

The terms “protein,” “peptide,” and “polypeptide” are used interchangeably herein.

The term “promoter” refers to a polynucleotide, usually upstream (5′) of its coding polynucleotide, which controls the expression of the coding polynucleotide by providing the recognition for RNA polymerase and other factors required for proper transcription.

“Constitutive promoter” refers to a promoter that is able to express the open reading frame (ORF) that it controls in all or nearly all of the plant tissues during all or nearly all developmental stages of the plant (referred to as “constitutive expression”). “Regulated promoter” refers to promoters that direct gene expression not constitutively, but in a temporally- and/or spatially-regulated manner, and includes tissue-specific, tissue-preferred and inducible promoters. It includes natural and synthetic polynucleotides as well as polynucleotides which may be a combination of synthetic and natural polynucleotides. Different promoters may direct the expression of a gene in different tissues or cell types, or at different stages of development, or in response to different environmental conditions.

As used herein, gene or trait “stacking” is combining desired genes or traits into one transgenic plant line. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. Another way to stack genes is by re-transforming a transgenic plant with another gene of interest. For example, gene stacking can be used to combine two different insect resistance traits, an insect resistance trait and a disease resistance trait, or an herbicide resistance trait (such as, for example, Bt11). The use of a selectable marker in addition to a gene of interest would also be considered gene stacking.

“Tissue-specific promoter” or “tissue-preferred promoter” refers to regulated promoters that are not expressed in all plant cells but only or preferentially in one or more cell types in specific organs (such as leaves or seeds), specific tissues (such as embryo or cotyledon), or specific cell types (such as leaf parenchyma or seed storage cells). These terms also include promoters that are temporally regulated, such as in early or late embryogenesis, during fruit ripening in developing seeds or fruit, in fully differentiated leaf, or at the onset of senescence. Those skilled in the art will understand that tissue-specific promoters need not exhibit an absolute tissue-specificity, but mediate transcriptional activation in most plant parts at a level of about 1% or less of the level reached in the part of the plant in which transcription is most active.

“Inducible promoter” refers to those regulated promoters that can be turned on in one or more cell types by an external stimulus, such as a chemical, light, hormone, stress, or a pathogen.

The terms “stringent conditions” or “stringent hybridization conditions” include reference to conditions under which a polynucleotide will hybridize to its target sequence to a detectably greater degree than other sequences (e.g., at least 2-fold over background). Stringent conditions are sequence-dependent and will be different in different circumstances. By controlling the stringency of the hybridization and/or washing conditions, target polynucleotides can be identified which are 100% complementary to the probe (homologous probing). Alternatively, stringency conditions can be adjusted to allow some mismatching in sequences so that lower degrees of similarity are detected (heterologous probing). Typically, stringent conditions will be those in which the salt concentration is less than approximately 1.5 M Na ion, typically about 0.01 to 1.0 M Na ion (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions also may be achieved with the addition of destabilizing agents such as formamide. Exemplary low stringency conditions include hybridization with a buffer solution of 30 to 35% formamide, 1 M NaCl, 1% SDS (w/v; sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA—DNA hybrids, the Tm can be approximated from the equation of Meinkoth and Wahl (Anal. Biochem., 138:267-284, 1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., eds., Greene Publishing and Wiley-Interscience, New York (1995). Methods of stringent hybridization are known in the art which conditions can be calculated by means known in the art. This is disclosed in Sambrook et al., Molecular Cloning: A Laboratory Manual, 2nd Ed., Cold Spring Harbor Laboratory Press, 1989, Cold Spring Harbor, N.Y. and Current Protocols in Molecular Biology, Ausebel et al, eds., John Wiley and Sons, Inc., 2000. Methods of determining percent sequence identity are known in the art, an example of which is the GCG computer sequence analysis software (GCG, Inc, Madison Wis.).

### DETAILED DESCRIPTION

The disclosure provides, at least in part, methods and compositions for improving transformation efficiency, using a WOX protein (e.g., WOX5), a BABY BOOM protein, or a combination thereof. Example WOX proteins and BABY BOOM proteins, and corresponding coding sequences, are described herein (see, e.g., the Sequence Listing, the Brief Description of the Sequences and Table 1).

One embodiment of the invention is a method, comprising transforming a plant with a nucleic acid encoding the amino acid sequence set forth in SEQ ID NO: 143, 27, 55, 93, 123, or 162, or a nucleic acid encoding a polypeptide comprising an amino acid sequence having an at least 85% identity (e.g., at least 85%, at least 90%, at least 95%, at least 98% or at least 99% identity) with the amino acid sequence set forth in SEQ ID NO: 143, 27, 55, 93, 123, or 162, and, optionally, having an effect that improves transformation efficiency of a plant. In another embodiment, the method comprises overexpressing in a plant an amino acid sequence having an at least 85% identity (e.g., at least 85%, at least 90%, at least 95%, at least 98%, at least 99% identity or 100% identity) with the amino acid sequence set forth in SEQ ID NO: 143, 27, 55, 93, 123, or 162, optionally wherein transformation efficiency of the plant is improved. In some embodiments, the nucleic acid encoding the amino acid sequence is a nucleic acid having a nucleic acid sequence of SEQ ID NO: 142, 26, 54, 92, 122, or 161, or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161. In some embodiments of the method, the plant is a monocotyledon, and it may be corn (i.e., maize), wheat, barley, rice, sorghum, and rye. In another aspect of the method, the plant is a dicotyledon, and it may be soybean, sunflower, watermelon, or Arabidopsis. In another embodiment of the method the improvement of transformation efficiency of a plant comprises one or more of: (i) improvement of efficiency of callus formation of the plant; (ii) improvement of redifferentiation rate of the plant; and (iii) improvement of gene transfer efficiency. In some embodiments, the method further comprises transforming the plant with a desired nucleic acid to be produced in the plant.

In another embodiment, the invention provides a method, comprising transforming a plant with a nucleic acid encoding a BABY BOOM amino acid sequence, optionally wherein transformation efficiency of the plant is improved. In another embodiment, the method comprises overexpressing in a plant a BABY BOOM amino acid sequence, optionally wherein transformation efficiency of the plant is improved. In some embodiments, the BABY BOOM amino acid sequence is an amino acid sequence having an at least 85% identity (e.g., at least 85%, at least 90%, at least 95%, at least 98%, at least 99% identity or 100% identity) with a BABY BOOM amino acid sequence. In some embodiments, the BABY BOOM amino acid sequence comprises an amino acid sequence of any one of SEQ ID NO: 205-227 or an amino acid sequence having an at least 85% identity (e.g., at least 85%, at least 90%, at least 95%, at least 98% or at least 99% identity) with an amino acid sequence of any one of SEQ ID NO: 205-227. In some embodiments, the BABY BOOM amino acid sequence comprises an amino acid sequence of any one of SEQ ID NO: 205, 211 or 213 or an amino acid sequence having an at least 85% identity (e.g., at least 85%, at least 90%, at least 95%, at least 98% or at least 99% identity) with an amino acid sequence of any one of SEQ ID NO: 205, 211 or 213. In some embodiments, the BABY BOOM amino acid sequence comprises an amino acid sequence of any one of SEQ ID NO: 205, 211, 213, or 224 or an amino acid sequence having an at least 85% identity (e.g., at least 85%, at least 90%, at least 95%, at least 98% or at least 99% identity) with an amino acid sequence of any one of SEQ ID NO: 205, 211, 213, or 224. In some embodiments, the nucleic acid encoding a BABY BOOM amino acid sequence is a nucleic acid having a nucleic acid sequence of any one of SEQ ID NO: 179-204 or a nucleic acid sequence having an at least 85% identity (e.g., at least 85%, at least 90%, at least 95%, at least 98% or at least 99% identity) with a nucleic acid sequence of any one of SEQ ID NO: 179-204. In some embodiments, the nucleic acid encoding a BABY BOOM amino acid sequence is selected from the group consisting of SEQ ID NO: 179, SEQ ID NO: 180, and SEQ ID NO: 181 or a nucleic acid sequence having an at least 85% identity (e.g., at least 85%, at least 90%, at least 95%, at least 98% or at least 99% identity) with a nucleic acid sequence of any one of SEQ ID NO: 179, SEQ ID NO: 180, and SEQ ID NO: 181. In some embodiments, the method further comprises transforming the plant with a desired nucleic acid to be produced in the plant.

In another embodiment, the invention provides a method, comprising transforming a plant with a nucleic acid encoding a WOX amino acid sequence (e.g., a WOX5 amino acid sequence) and a nucleic acid encoding a BABY BOOM amino acid sequence, optionally wherein transformation efficiency of the plant is improved. In another embodiment, the method comprises overexpressing in a plant a WOX amino acid sequence (e.g., a WOX5 amino acid sequence) and a BABY BOOM amino acid sequence, optionally wherein transformation efficiency of the plant is improved. In some embodiments, the WOX amino acid sequence comprises the amino acid sequence set forth in SEQ ID NO: 143, 27, 55, 93, 123, or 162, or an amino acid sequence having an at least 85% identity (e.g., at least 85%, at least 90%, at least 95%, at least 98% or at least 99% identity) with the amino acid sequence set forth in SEQ ID NO: 143, 27, 55, 93, 123, or 162, and the BABY BOOM amino acid sequence comprises an amino acid sequence of any one of SEQ ID NO: 205-227 or an amino acid sequence having an at least 85% identity (e.g., at least 85%, at least 90%, at least 95%, at least 98% or at least 99% identity) with an amino acid sequence of any one of SEQ ID NO: 205-227. In some embodiments, the nucleic acid encoding a WOX amino acid sequence is a nucleic acid having a nucleic acid sequence of SEQ ID NO: 142, 26, 54, 92, 122, or 161, or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161. In some embodiments, the BABY BOOM amino acid sequence comprises an amino acid sequence of any one of SEQ ID NO: 205, 211 or 213 or an amino acid sequence having an at least 85% identity (e.g., at least 85%, at least 90%, at least 95%, at least 98% or at least 99% identity) with an amino acid sequence of any one of SEQ ID NO: 205, 211 or 213. In some embodiments, the BABY BOOM amino acid sequence comprises an amino acid sequence of any one of SEQ ID NO: 205, 211, 213, or 224 or an amino acid sequence having an at least 85% identity (e.g., at least 85%, at least 90%, at least 95%, at least 98% or at least 99% identity) with an amino acid sequence of any one of SEQ ID NO: 205, 211, 213, or 224. In some embodiments, the nucleic acid encoding a BABY BOOM amino acid sequence is a nucleic acid having a nucleic acid sequence of any one of SEQ ID NO: 179-204 or a nucleic acid sequence having an at least 85% identity (e.g., at least 85%, at least 90%, at least 95%, at least 98% or at least 99% identity) with a nucleic acid sequence of any one of SEQ ID NO: 179-204. In some embodiments, the nucleic acid encoding a BABY BOOM amino acid sequence is selected from the group consisting of SEQ ID NO: 179, SEQ ID NO: 180, and SEQ ID NO: 181 or a nucleic acid sequence having an at least 85% identity (e.g., at least 85%, at least 90%, at least 95%, at least 98% or at least 99% identity) with a nucleic acid sequence of any one of SEQ ID NO: 179, SEQ ID NO: 180, and SEQ ID NO: 181. In some embodiments, the method further comprises transforming the plant with a desired nucleic acid to be produced in the plant.

Another embodiment of the invention is a nucleic acid construct comprising: (i) a nucleic acid encoding the amino acid sequence set forth in SEQ ID NO: 143, 27, 55, 93, 123, or 162, or a nucleic acid encoding a polypeptide comprising an amino acid sequence having at least 85% identity (e.g., at least 85%, at least 90%, at least 95%, at least 98%, at least 99% identity, or 100% identity) with the amino acid sequence set forth in SEQ ID NO: 143, 27, 55, 93, 123, or 162, and optionally having an effect that improves transformation efficiency of a plant; and (ii) a promoter for producing a nucleic acid in the plant. In some embodiments, the nucleic acid encoding the amino acid sequence is a nucleic acid having a nucleic acid sequence of SEQ ID NO: 142, 26, 54, 92, 122, or 161, or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161. In some embodiments, the promoter is a constitutive promoter, an inducible promoter, or a site-specific promoter. In another embodiment of the method comprises introducing into a plant a nucleic acid construct above, and further comprising a second nucleic acid to be expressed in the plant. In some embodiments, the transformation is transient. In another, it is stable. Another embodiment is a transformed plant obtained by the method of transformation.

Yet another embodiment of the invention is a nucleic acid construct comprising: (i) a nucleic acid encoding the amino acid sequence set forth in SEQ ID NO: 143, 27, 55, 93, 123, or 162 or a nucleic acid encoding a polypeptide comprising an amino acid sequence having at least 85% identity (e.g., at least 85%, at least 90%, at least 95%, at least 98% or at least 99% identity) with the amino acid sequence set forth in SEQ ID NO: 143, 27, 55, 93, 123, or 162; and (ii) a promoter for producing a nucleic acid in a plant; optionally wherein the transformation efficiency is improved. In some embodiments, the nucleic acid encoding the amino acid sequence is a nucleic acid having a nucleic acid sequence of SEQ ID NO: 142, 26, 54, 92, 122, or 161, or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161. In a further aspect, the nucleic acid construct further comprises a desired nucleic acid to be produced in the plant.

In another embodiment, the invention provides a method, comprising transforming a plant with (a) a nucleic acid encoding the amino acid sequence set forth in SEQ ID NO: 143, 27, 55, 93, 123, or 162 or a nucleic acid encoding a polypeptide comprising an amino acid sequence having an at least 85% identity (e.g., at least 85%, at least 90%, at least 95%, at least 98% or at least 99% identity) with the amino acid sequence set forth in SEQ ID NO: 143, 27, 55, 93, 123, or 162; and (b) a nucleic acid encoding a BABY BOOM amino acid sequence; optionally wherein the transformation efficiency of a plant is improved compared to a wildtype plant. In some embodiments, the nucleic acid encoding the amino acid sequence is a nucleic acid having a nucleic acid sequence of SEQ ID NO: 142, 26, 54, 92, 122, or 161, or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161. In some embodiments, the BABY BOOM amino acid sequence comprises an amino acid sequence of any one of SEQ ID NO: 205-227 or an amino acid sequence having an at least 85% identity (e.g., at least 85%, at least 90%, at least 95%, at least 98% or at least 99% identity) with an amino acid sequence of any one of SEQ ID NO: 205-227. In some embodiments, the BABY BOOM amino acid sequence comprises an amino acid sequence of any one of SEQ ID NO: 205, 211 or 213 or an amino acid sequence having an at least 85% identity (e.g., at least 85%, at least 90%, at least 95%, at least 98% or at least 99% identity) with an amino acid sequence of any one of SEQ ID NO: 205, 211 or 213. In some embodiments, the BABY BOOM amino acid sequence comprises an amino acid sequence of any one of SEQ ID NO: 205, 211, 213, or 224 or an amino acid sequence having an at least 85% identity (e.g., at least 85%, at least 90%, at least 95%, at least 98% or at least 99% identity) with an amino acid sequence of any one of SEQ ID NO: 205, 211, 213, or 224. In some embodiments, the nucleic acid encoding a BABY BOOM amino acid sequence is a nucleic acid having a nucleic acid sequence of any one of SEQ ID NO: 179-204 or a nucleic acid sequence having an at least 85% identity (e.g., at least 85%, at least 90%, at least 95%, at least 98% or at least 99% identity) with a nucleic acid sequence of any one of SEQ ID NO: 179-204. In some embodiments, the nucleic acid encoding a BABY BOOM amino acid sequence is selected from the group consisting of SEQ ID NO: 179, SEQ ID NO: 180, and SEQ ID NO: 181 or a nucleic acid sequence having an at least 85% identity (e.g., at least 85%, at least 90%, at least 95%, at least 98% or at least 99% identity) with a nucleic acid sequence of any one of SEQ ID NO: 179, SEQ ID NO: 180, and SEQ ID NO: 181. In some embodiments, the method further comprises transforming the plant with a desired nucleic acid to be produced in the plant.

TABLE l
Example BABY BOOM genes

| | | | Nucleic | Amino |
|---|---|---|---|---|
| | | | Acid | Acid |
| | | | sequence | sequence |
| | Gene | | SEQ ID | SEQ ID |
| Plant Species | name | Gene ID | NO | NO |
| Selaria italica | SiBBM1 | Seita.5G415800.l | 182 | 205 |
| Panicum | PvBBM1 | Pavir.J01327.1 | 183 | 206 |
| virgatum | | | | |
| Panicum | PvBBM2 | Pavir.Ea03550.1 | 184 | 207 |
| virgatum | | | | |
| Oryza sativa | OsBBM1 | Os11g19060 | 185 | 208 |
| Oryza sativa | OsBBM2 | Os02g40070 | 186 | 209 |
| Oryza sativa | OsBBM3 | Os01g67410 | 187 | 210 |
| Brachypodium | BdBBM1 | Bradi2g57747.2 | 188 | 211 |
| distachyon | | | | |
| Brassica | BnBBM2 | AF317905.1 | 189 | 212 |
| napus | | | | |
| Brassica | BnBBM1 | AF317904.1 | 190 | 213 |
| napus | | | | |
| Brassica rapa | BrBBM1 | Brara.J01807.1 | 191 | 214 |
| Brassica rapa | BrBBM2 | Brara.B00712.1 | 192 | 215 |
| Boechera | BsBBM | Bostr.26527s0471.1 | 193 | 216 |
| stricta | | | | |
| Capsella | CrBBM | Carubv10002745m | 194 | 217 |
| rubella | | | | |
| Capsella | CgBBM | Cagra.6170s0013.1 | 195 | 218 |
| grandiflora | | | | |
| Setaria viridis | SvBBM1 | Sevir.5G421100.1 | 196 | 219 |
| Zea mays | ZmODP2 | CS155772.1 | 197 | 220 |
| Zea mays | ZmBBM | GRMZM2G141638_ | 198 | 221 |
| | | T01 | | |
| Pennisetum | PsBBML | EU559280 | 199 | 222 |
| squamulatum | | | | |
| Arabidopsis | AtBBM | AF317907.1 | 200 | 223 |
| thaliana | | | | |
| Setaria italica | SiBBM2 | XP_004979180.1 | 201 | 224 |
| Panicum | PvBBM3 | Pavir.Hb01059.1 | 202 | 225 |
| virgatum | | | | |
| Panicum | PhBBM2 | Pahal.H02285.1 | 203 | 226 |
| hallii | | | | |
| Panicum | PhBBM1 | Pahal.E00418.1 | 204 | 227 |
| hallii | | | | |

In another embodiment, the invention provides a nucleic acid construct comprising: (a) a nucleic acid encoding the amino acid sequence set forth in SEQ ID NO: 143, 27, 55, 93, 123, or 162 or a nucleic acid encoding a polypeptide comprising an amino acid sequence having at least 85% identity (e.g., at least 85%, at least 90%, at least 95%, at least 98% or at least 99% identity) with the amino acid sequence set forth in SEQ ID NO: 143, 27, 55, 93, 123, or 162; (b) a nucleic acid encoding a BABY BOOM amino acid sequence; and (c) a promoter for producing a nucleic acid of (a) and (b) in the plant; optionally wherein the transformation efficiency is improved. In another embodiment, the nucleic acid construct according further comprising a desired nucleic acid to be produced in the plant. In some embodiments, the nucleic acid encoding the amino acid sequence is a nucleic acid having a nucleic acid sequence of SEQ ID NO: 142, 26, 54, 92, 122, or 161, or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161. In some embodiments, the BABY BOOM amino acid sequence comprises an amino acid sequence of any one of SEQ ID NO: 205-227 or an amino acid sequence having an at least 85% identity (e.g., at least 85%, at least 90%, at least 95%, at least 98% or at least 99% identity) with an amino acid sequence of any one of SEQ ID NO: 205-227. In some embodiments, the BABY BOOM amino acid sequence comprises an amino acid sequence of any one of SEQ ID NO: 205, 211 or 213 or an amino acid sequence having an at least 85% identity (e.g., at least 85%, at least 90%, at least 95%, at least 98% or at least 99% identity) with an amino acid sequence of any one of SEQ ID NO: 205, 211 or 213. In some embodiments, the BABY BOOM amino acid sequence comprises an amino acid sequence of any one of SEQ ID NO: 205, 211, 213, or 224 or an amino acid sequence having an at least 85% identity (e.g., at least 85%, at least 90%, at least 95%, at least 98% or at least 99% identity) with an amino acid sequence of any one of SEQ ID NO: 205, 211, 213, or 224. In some embodiments, the nucleic acid encoding a BABY BOOM amino acid sequence is a nucleic acid having a nucleic acid sequence of any one of SEQ ID NO: 179-204 or a nucleic acid sequence having an at least 85% identity (e.g., at least 85%, at least 90%, at least 95%, at least 98% or at least 99% identity) with a nucleic acid sequence of any one of SEQ ID NO: 179-204. In some embodiments, the nucleic acid construct comprises nucleic acid encoding a BABY BOOM amino acid sequence selected from the group consisting of SEQ ID NO: 179, SEQ ID NO: 180, and SEQ ID NO: 181 or a nucleic acid sequence having an at least 85% identity (e.g., at least 85%, at least 90%, at least 95%, at least 98% or at least 99% identity) with a nucleic acid sequence of any one of SEQ ID NO: 179, SEQ ID NO: 180, and SEQ ID NO: 181.

The invention provides in another embodiment a nucleic acid construct comprising SEQ ID NO: 179, 180 or 181 operably linked to a heterologous regulatory sequence. Also provided is a method, comprising transforming a plant with a nucleic acid comprising a sequence set forth in SEQ ID NO: 179, 180, or 181 or a nucleic acid sequence having an at least 85% identity (e.g., at least 85%, at least 90%, at least 95%, at least 98% or at least 99% identity) with the sequence set forth in SEQ ID NO: 179, 180, or 181; optionally wherein the transformation efficiency of a plant is improved compared to a wildtype plant. In another embodiment, the invention provides a method for producing a haploid plant comprising (a) transiently transforming a plant cell with a nucleic acid encoding a WOX amino acid sequence (e.g., a WOX5 amino acid sequence) under the control of a promoter to produce a transgenic plant cell, wherein the promoter is selected from the group consisting of a haploid tissue specific promoter, an inducible promoter and a promoter that is both haploid-tissue specific and inducible; (b) optionally transforming the plant cell with a nucleic acid sequence encoding a BABY BOOM amino acid sequence; (c) generating a transgenic plant from said transgenic plant cell; (d) overexpressing the nucleic acid encoding the WOX amino acid sequence in a haploid tissue of said transgenic plant to produce a haploid somatic embryo; and (e) growing said embryo into a haploid plant. In some embodiments, the WOX amino acid sequence comprises the amino acid sequence set forth in SEQ ID NO: 143, 27, 55, 93, 123, or 162, or an amino acid sequence having an at least 85% identity (e.g., at least 85%, at least 90%, at least 95%, at least 98% or at least 99% identity) with the amino acid sequence set forth in SEQ ID NO: 143, 27, 55, 93, 123, or 162, and the BABY BOOM amino acid sequence comprises an amino acid sequence of any one of SEQ ID NO: 205-227 or an amino acid sequence having an at least 85% identity (e.g., at least 85%, at least 90%, at least 95%, at least 98% or at least 99% identity) with an amino acid sequence of any one of SEQ ID NO: 205-227. In some embodiments, the nucleic acid encoding a WOX amino acid sequence is a nucleic acid having a nucleic acid sequence of SEQ ID NO: 142, 26, 54, 92, 122, or 161, or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161. In some embodiments, the BABY BOOM amino acid sequence comprises an amino acid sequence of any one of SEQ ID NO: 205, 211 or 213 or an amino acid sequence having an at least 85% identity (e.g., at least 85%, at least 90%, at least 95%, at least 98% or at least 99% identity) with an amino acid sequence of any one of SEQ ID NO: 205, 211 or 213. In some embodiments, the BABY BOOM amino acid sequence comprises an amino acid sequence of any one of SEQ ID NO: 205, 211, 213, or 224 or an amino acid sequence having an at least 85% identity (e.g., at least 85%, at least 90%, at least 95%, at least 98% or at least 99% identity) with an amino acid sequence of any one of SEQ ID NO: 205, 211, 213, or 224. In some embodiments, the nucleic acid encoding a BABY BOOM amino acid sequence is a nucleic acid having a nucleic acid sequence of any one of SEQ ID NO: 179-204 or a nucleic acid sequence having an at least 85% identity (e.g., at least 85%, at least 90%, at least 95%, at least 98% or at least 99% identity) with a nucleic acid sequence of any one of SEQ ID NO: 179-204. In some embodiments, the nucleic acid encoding a BABY BOOM amino acid sequence is selected from the group consisting of SEQ ID NO: 179, SEQ ID NO: 180, and SEQ ID NO: 181 or a nucleic acid sequence having an at least 85% identity (e.g., at least 85%, at least 90%, at least 95%, at least 98% or at least 99% identity) with a nucleic acid sequence of any one of SEQ ID NO: 179, SEQ ID NO: 180, and SEQ ID NO: 181.

In another embodiment, the invention provides a method for producing a haploid plant comprising (a) transiently transforming a plant cell with a nucleic acid encoding the amino acid sequence set forth in SEQ ID NO: 143, 27, 55, 93, 123, or 162, or an amino acid sequence having an at least 85% identity (e.g., at least 85%, at least 90%, at least 95%, at least 98% or at least 99% identity) with the amino acid sequence set forth in SEQ ID NO: 143, 27, 55, 93, 123, or 162, under the control of a promoter to produce a transgenic plant cell, wherein the promoter is selected from the group consisting of a haploid tissue specific promoter, an inducible promoter and a promoter that is both haploid-tissue specific and inducible; (b) optionally transforming the plant cell with a nucleic acid sequence encoding a BABY BOOM amino acid sequence, wherein the nucleic acid sequence is selected from the group consisting of SEQ ID NO: 179, SEQ ID NO: 180, and SEQ ID NO: 181 or a nucleic acid sequence having an at least 85% identity (e.g., at least 85%, at least 90%, at least 95%, at least 98% or at least 99% identity) with a nucleic acid sequence of any one of SEQ ID NO: 179, SEQ ID NO: 180, and SEQ ID NO: 181; (c) generating a transgenic plant from said transgenic plant cell; (d) overexpressing the nucleic acid encoding the amino acid sequence set forth in SEQ ID NO: 143, 27, 55, 93, 123, or 162, or the amino acid sequence having an at least 85% identity (e.g., at least 85%, at least 90%, at least 95%, at least 98% or at least 99% identity) with the amino acid sequence set forth in SEQ ID NO: 143, 27, 55, 93, 123, or 162 in a haploid tissue of said transgenic plant to produce a haploid somatic embryo; and (e) growing said embryo into a haploid plant. In some embodiments, the nucleic acid encoding the amino acid sequence is a nucleic acid having a nucleic acid sequence of SEQ ID NO: 142, 26, 54, 92, 122, or 161 or a nucleic acid sequence having an at least 85% identity (e.g., at least 85%, at least 90%, at least 95%, at least 98% or at least 99% identity) with a nucleic acid sequence of SEQ ID NO: 142, 26, 54, 92, 122, or 161.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the promoter is an egg-cell preferred promoter.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the promoter is SEQ ID NO. 288.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the plant is a monocotyledon.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the monocotyledon is corn.

In another embodiment, the invention provides a method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant, wherein the plant comprises the matrilineal haploid induction locus.

In another embodiment, the invention provides a haploid plant obtained by the method for producing a haploid plant comprising (a) transiently transforming a plant cell with a nucleic acid sequence from group consisting of SEQ ID NO: 179, SEQ ID NO: 180, and SEQ ID NO: 181 under the control of a promoter to produce a transgenic plant cell, wherein the promoter is selected from the group consisting of a haploid tissue specific promoter, an inducible promoter and a promoter that is both haploid-tissue specific and inducible; (b) generating a transgenic plant from said transgenic plant cell; (c) overexpressing the nucleic acid encoding the amino acid sequence set forth in SEQ ID NO: 162, SEQ ID NO: 229 or SEQ ID NO: 230, in a haploid tissue of said transgenic plant to produce a haploid somatic embryo; and (e) growing said embryo into a haploid plant.

In another embodiment, the invention provides a recombinant DNA molecule comprising a DNA sequence selected from the group consisting of: a) a sequence with at least 85 percent sequence identity to SEQ ID NO:288; b) a fragment of SEQ ID NO:288, wherein the fragment has gene-regulatory activity; wherein said DNA sequence is operably linked to a heterologous transcribable DNA molecule.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant comprises the matrilineal haploid induction locus.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant comprises modifications to alter meiosis to mitosis.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant comprises modifications to alter meiosis to mitosis, wherein the plant comprises knockouts of the meiotic genes REC8, PAIR1, and OSD1.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the promoter is an egg-cell preferred promoter.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the promoter is an egg-cell preferred promoter, wherein the promoter is SEQ ID NO. 288.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant is a monocotyledon.

In another embodiment, the invention provides a method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization, wherein the plant is a monocotyledon, wherein the monocotyledon is corn.

In another embodiment, the invention provides a plant produced by the method of propagating from one or more gametophytic or sporophytic cells in an ovule of a plant in the absence of egg cell fertilization, the method comprising:

transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and growing and selecting a progeny plant from the one or more gametophytic or sporophytic cells, wherein the progeny plant contains one or more sets of chromosomes from the transformed plant, and wherein propagation of the plant occurs in the absence of egg cell fertilization.

This invention further provides, in some embodiments, plants or plant parts (e.g., transformed plants or plant parts) produced by any of the methods described herein.

AX5707RS is a transformable haploid inducer with AX5707 background containing the matrilineal gene mutation (mat1) and Rscm2 color marker. MATRILINEAL, a sperm-specific phospholipase, triggers maize haploid induction described in Kelliher et al. Nature 2017 Feb. 2; 542(7639):105-109 herein incorporated by reference.

The examples below will aid a person having ordinary skill in the art understand the scope pf these embodiments.

### EXAMPLES

### Example 1

Three independent maize transformation experiments were performed with the SbWOX5 coding sequence (SEQ ID NO: 142) which encodes the SbWOX5 protein (SEQ ID NO: 143) and, as a control, without the gene. The SbWOX5 coding sequence was driven by the Nopaline synthetase gene promoter from Agrobacterium tumefaciens Ti plasmid (prNOS, EMBL: 212288). In experiments combined with a Baby Boom coding sequence (one of SEQ ID NO: 179, SEQ ID NO: 180 and SEQ ID NO: 181), the Baby Boom coding sequence was driven by a Maize ubiquitin 1 gene promoter (prUbi1). In preparation of transformation, maize explants were surface sterilized with a solution comprising Tween-20 and 20% bleach.

Transgenic maize events were generated using Agrobacterium-mediated transformation of 6 commercially important inbred maize lines Inbred 1, Inbred 2, Inbred 3, Inbred 4, Inbred 5, and Inbred 6. These lines are highly recalcitrant to transformation and regeneration with available methods; yet were used to evaluate the morphogenetic regulator genes, including SbWOX5. Agrobacterium-mediated transformation was conducted as outlined in Negrotto, et al. (Plant Cell Report 19:798-803, 2000; incorporated herein by reference) and Zhong, H., et al. (2018).

Isolation of immature embryo was performed accordingly to the methods described in Zhong, H et al. 2018. Isolated immature embryos sized ranging from 0.7 to 1.2 mm from the sterilized corn stock ears were resuspended in infection liquid then inoculated with Agrobacterium. After infection, explants were placed on co-cultivation medium by incubating it at 23° C. in the dark for 2-3 days. After a period of co-cultivation, explants were transferred to recovery medium supplemented with silver nitrate (10 mg/L) and Timentin (100-200 mg/L) to inhibit or kill Agrobacterium and at the same time allow plant cells to grow and recover.

Recovered explants were transferred to fresh selection media to allow only the transformed plant cells to grow preferentially in the presence or absence of a selection agent mannose. This step helped to differentiate the transformed cells from untransformed cells. Healthy transformed calli were selected and desiccated for 1-2 days on WHATMAN® filter paper to activate CRE-lox excision system which is under the control of rap17 promoter. Then the desiccated transgenic calli were transferred to fresh regeneration media supplemented either with or without selection agent mannose to allow putative transformed callus lines to produce shoots. Regenerated shoots were then transferred to a rooting medium for shoot rooting and elongation to establish well rooted plantlets. When ready, plants were sampled for TAQMAN® qPCR analysis to detect the presence of transgene. Plants positive for transgene were confirmed and the desired plants were transferred to greenhouse for further propagation and seed set. Results are shown in Table 2.

Outline of transformation workflow:
1. Isolation and Inoculation of immature embryos with Agrobacterium.
2. Co-Cultivation (2-3 days).
3. Recovery or callus induction (14-21 days depending on genotype).
4. Selection 1 (14 days).
5. Selection 2 (14 days).
6. Callus desiccation (1-2 days on a pre-sterilized Whatman® filter paper).
7. Regeneration 1 (in dark for 14 days).
8. Regeneration 2 (in light for 14 days).
   a. Medium for Regeneration 1 and 2 is unchanged; the boxes are merely moved from dark to light.
9. Rooting (10-14 days).
10. PCR analyses.
11. Positive and desired plants sent to greenhouse for further propagation and seed set.

TABLE 2
Transformation efficiency of SbW0X5 (SEQ ID NO: 143) used in
various maize genotypes.

| | | | Number | | |
|---|---|---|---|---|---|
| | | | of | Number | |
| Construct | WUS/BBM | Genotype | explants | of | Transformation |
| ID | orthologs | tested | used | events | frequency % |
| 12672 | Control | Inbred 1 | 407 | 7 | 1.75 |
| | (PMI + | Inbred 2 | 470 | 0 | 0.00 |
| | CFP) | Inbred 3 | 147 | 0 | 0.00 |
| | | Inbred 4 | 241 | 0 | 0.00 |
| | | Inbred 5 | 100 | 0 | 0.00 |
| | | Inbred 6 | 135 | 1 | 0.74 |
| 23958 | prNOS- | Inbred 1 | 290 | 56 | 17.50 |
| | SbWOX5 + | Inbred 2 | 334 | 44 | 13.90 |
| | prUbi1- | Inbred 3 | 247 | 9 | 3.64 |
| | SiBBM1 | Inbred 5 | 460 | 149 | 32.39 |
| | (SEQID | Inbred 6 | 170 | 13 | 7.65 |
| | NO: 179) | | | | |
| 23966 | prNOS- | Inbred 1 | 297 | 84 | 27.95 |
| | SbWOX5 + | Inbred 2 | 293 | 20 | 6.83 |
| | prUbi1- | Inbred 3 | 105 | 4 | 3.81 |
| | BdBBM1 | Inbred 4 | 134 | 9 | 6.72 |
| | (SEQID | Inbred 5 | 300 | 169 | 56.33 |
| | NO: 180) | Inbred 6 | 252 | 76 | 30.16 |
| 23967 | prNOS- | Inbred 1 | 291 | 84 | 26.15 |
| | SbWOX5 + | Inbred 2 | 460 | 71 | 15.92 |
| | prUbi1- | Inbred 3 | 200 | 21 | 10.50 |
| | BnBBM1 | Inbred 5 | 399 | 190 | 47.62 |
| | (SEQID | Inbred 6 | 117 | 51 | 43.59 |
| | NO: 181) | | | | |

### Example 2

Experiments were carried out to test the transformation enhancing effect of several Brachypodium WOX homologs with or without BnBBM or BdBBM (Table 3). BdWOX5 was shown to improve recalcitrant corn AA3676 transformation (vector 25072) when under the control of strong constitutive maize ubiquitin 1 promoter (prZmUbil). Use of BdWOX5 in transformation does not require Cre-loxP-mediated excision of the morphogenic factor gene BBM and WOX cassettes like in 23958, 23966 and 23967. Therefore, it is more straightforward and simpler to use in transformation studies. Also, it is easier to make transformation vectors since there is no need to include Cre and BBM expression cassettes.

TABLE 3
Further vectors for enhancing transformation and haploid induction efficiency

| | | | | | | | Number | | |
|---|---|---|---|---|---|---|---|---|---|
| | | Cassette1 | | | Cassette 4 | | of | Number | |
| Vector | Vector | (Cre or | Cassette2 | Cassette3 | (selectable | Genotype | explants | of | Transformation |
| # | ID | CFP) | (WOX) | (BBM) | marker) | tested | used | events | frequency % |
| 1 | 25118 | | prZmUbi1- | | prZmUbi1- | AX5707 | 440 | 33 | 7.5 |
| | | | BdWUS | | AtPPO | AA3676 | 490 | 0 | 0 |
| | | | prUbi- | | | | | | |
| | | | BdWUS | | | | | | |
| | | | (SEQ ID | | | | | | |
| | | | NO: 161 | | | | | | |
| | | | coding | | | | | | |
| | | | sequence, | | | | | | |
| | | | SEQ ID | | | | | | |
| | | | NO: 162 | | | | | | |
| | | | protein | | | | | | |
| | | | sequence) | | | | | | |
| 2 | 25070 | | prZmUbi1- | | prZmUbi1- | AX5707 | 360 | 16 | 4.4 |
| | | | BdWOX2 | | AtPPO | AA3676 | 570 | 0 | 0 |
| | | | (SEQ ID | | | | | | |
| | | | NO: 26 | | | | | | |
| | | | coding | | | | | | |
| | | | sequence, | | | | | | |
| | | | SEQ ID | | | | | | |
| | | | NO: 27 | | | | | | |
| | | | protein | | | | | | |
| | | | sequence) | | | | | | |
| 3 | 25076 | | prZmUbi1- | | prZmUbi1- | AX5707 | 200 | 0 | 0 |
| | | | BdWOX3 | | AtPPO | AA3676 | 575 | 2 | 0.3 |
| | | | prUbi- | | | | | | |
| | | | BdWOX3 | | | | | | |
| | | | (SEQ ID | | | | | | |
| | | | NO: 54 | | | | | | |
| | | | coding | | | | | | |
| | | | sequence, | | | | | | |
| | | | SEQ ID | | | | | | |
| | | | NO: 55 | | | | | | |
| | | | protein | | | | | | |
| | | | sequence) | | | | | | |
| 4 | 25071 | | prZmUbi1- | | prZmUbi1- | AX5707 | 260 | 11 | 4.2 |
| | | | BdWOX4 | | AtPPO | AA3676 | 792 | 0 | 0 |
| | | | (SEQ ID | | | | | | |
| | | | NO: 92 | | | | | | |
| | | | coding | | | | | | |
| | | | sequence, | | | | | | |
| | | | SEQ ID | | | | | | |
| | | | NO: 93 | | | | | | |
| | | | protein | | | | | | |
| | | | sequence) | | | | | | |
| 5 | 25072 | | prZmUbi1- | | prZmUbi1- | AX5707 | 300 | 73 | 24 |
| | | | BdWOX5 | | AtPPO | AA3676 | 670 | 47 | 7 |
| | | | (SEQ ID | | | | | | |
| | | | NO: 122 | | | | | | |
| | | | coding | | | | | | |
| | | | sequence, | | | | | | |
| | | | SEQ ID | | | | | | |
| | | | NO: 123 | | | | | | |
| | | | protein | | | | | | |
| | | | sequence) | | | | | | |
| 6 | 25128 | prRab17- | prNOS- | prZmUbi1- | prZmUbi1- | AX5707 | 487 | 37 | 7.6 |
| | | CRE | BdWOX5 | BnBBM1 | AtPPO | AA3676 | 450 | 13 | 2.9 |
| | | | (SEQ ID | (SEQ ID | | | | | |
| | | | NO: 122 | NO: 190 | | | | | |
| | | | coding | coding | | | | | |
| | | | sequence, | sequence, | | | | | |
| | | | SEQ ID | SEQ ID | | | | | |
| | | | NO: 123 | NO: 213 | | | | | |
| | | | protein | protein | | | | | |
| | | | sequence) | sequence) | | | | | |
| 7 | 25127 | prRab17- | prNOS- | prZmUbi- | prZmUbi- | AX5707 | 340 | 5 | 1.5 |
| | | CRE | BdWOX5 | BdBBM1 | AtPPO | AA3676 | 600 | 2 | 0.3 |
| | | | (SEQ ID | (SEQ ID | | | | | |
| | | | NO: 122 | NO: 188 | | | | | |
| | | | coding | coding | | | | | |
| | | | sequence, | sequence, | | | | | |
| | | | SEQ ID | SEQ ID | | | | | |
| | | | NO: 123 | NO: 211 | | | | | |
| | | | protein | protein | | | | | |
| | | | sequence) | sequence) | | | | | |
| 8 | 25129 | prRab17- | | prZmUbi- | prZmUbi- | AX5707 | 207 | 10 | 4.8 |
| | | CRE | | BdBBM1 | AtPPO | AA3676 | 365 | 6 | 1.6 |
| | | | | (SEQ ID | | | | | |
| | | | | NO: 188 | | | | | |
| | | | | coding | | | | | |
| | | | | sequence, | | | | | |
| | | | | SEQ ID | | | | | |
| | | | | NO: 211 | | | | | |
| | | | | protein | | | | | |
| | | | | sequence) | | | | | |
| 9 | 25056 | | | prDsEc- | prAct-PMI | AX5707 | 612 | 262 | 43.3 |
| | | | | BdBBM1 | | AX5707RS | 116 | 26 | 22.4 |
| | | | | (SEQ ID | | | | | |
| | | | | NO: 188 | | | | | |
| | | | | coding | | | | | |
| | | | | sequence, | | | | | |
| | | | | SEQ ID | | | | | |
| | | | | NO: 211 | | | | | |
| | | | | protein | | | | | |
| | | | | sequence) | | | | | |
| 10 | 25115 | | prDsEc- | | prAct-PMI | AX5707 | 565 | 275 | 48.7 |
| | | | BdWUS | | | AX5707RS | 197 | 110 | 55.8 |
| | | | (SEQ ID | | | | | | |
| | | | NO: 161 | | | | | | |
| | | | coding | | | | | | |
| | | | sequence, | | | | | | |
| | | | SEQ ID | | | | | | |
| | | | NO: 162 | | | | | | |
| | | | protein | | | | | | |
| | | | sequence) | | | | | | |
| 11 | 25055 | prDsEC | | | prAct-PMI | AX5707 | 580 | 288 | 49.7 |
| | | (SEQ ID | | | | AX5707RS | 116 | 36 | 31 |
| | | NO. 228)- | | | | | | | |
| | | ZsGreen | | | | | | | |
| 12 | 25054 | | | prDsEC- | prAct-PMI | AX5707 | 538 | 242 | 45 |
| | | | | cSiBBM1 | | AX5707RS | 226 | 82 | 36.3 |
| | | | | (SEQ ID | | | | | |
| | | | | NO: 201 | | | | | |
| | | | | coding | | | | | |
| | | | | sequence, | | | | | |
| | | | | SEQ ID | | | | | |
| | | | | NO: 224 | | | | | |
| | | | | protein | | | | | |
| | | | | sequence) | | | | | |

### Example 3

BdWUS, BdBBM1 and SiBBM2 genes were also expressed under the control of egg-specific promoter (prDsEC) to test their effect on inducing haploid induction formation (Table 4). A control vector (prDsEC-ZsGreen) is used to confirm egg-specific expression driven by prDsEC promoter isolated from Boechera stricta (see Example 4). These vectors were transformed into maize immature embryos. Transgenic plants were assayed for the presence of transgene. Transgenic plants expressing BdWUS, BdBBM1, and SiBBM2 were also outcrossed to tester lines. Progeny plants were assayed for haploid chromosomes by genotyping assays for the transgenes and positive haploid plants are confirmed with ploidy level analysis using flow cytometry as described (see Kelliher, T. et al., 2019, One-step genome editing of elite crop germplasm during haploid induction. Nature Biotech. 37: 287-292). Some transgenic events of both BdBBM (MZET194504A051A and MZET194504A055A) and SiBBM (MZET194402B021A) expressors are able to induce high level of haploid plant formation when placed under the control of egg-cell specific promoter in corn; Haploid formation was observed only when transgene is provided from the female egg donor side, not from pollen donor, suggesting egg-cell preferred expression is critical for haploid formation. With limited experiments, we did not observe haploid induction with BdWUS gene. Successful haploid induction demonstrates that prDsEC drives expression of heterologous genes in the egg cell.

TABLE 4

| | | | | | Haploid |
|---|---|---|---|---|---|
| | | | | | induction rate |
| Morphogenic | Selectable | | T0 Event | | (haploids/total |
| factor gene | marker | T0 Event ID | background | Cross (Female/Pollen donor) | positives) |
| prDsEc- | prAct- | MZET194504A051A | AX5707 | MZET194504A051A/ID5829 | 37.5% |
| BdBBM1 | PMI | MZET194504A055A | AX5707 | MZET194504A055A/ID5829 | 45.5% |
| | | MZET194504A058A | AX5707 | MZET194504A058A/ID5829 | 0.0% |
| | | MZET194504A062A | AX5707 | ID5829/MZET194504A062A | 0.0% |
| prDsEC- | prAct- | MZET194505A065A | AX5707 | MZET194505A065A/ID5829 | 0.0% |
| cSiBBMl | PMI | MZET194402B021A | AX5707RS | MZET194402B021A/ID5829 | 82.9% |
| | | MZET194505B029A | AX5707 | ID5829/MZET194505B029A | 0.0% |
| | | MZET194505A098A | AX5707 | ID5829/MZET194505A098A | 0.0% |
| prDsEc- | prAct- | MZET194503A082A | AX5707 | MZET194503A082A/ID5829 | 0.0% |
| BdWUS | PMI | MZET194503A084A | AX5707 | MZET194503A084A/ID5829 | 0.0% |
| | | MZET194503A097A | AX5707 | ID5829/MZET194503A097A | 0.0% |
| | | MZET194503A092A | AX5707 | ID5829/MZET194503A092A | 0.0% |

Note:
T0 transgenic plants are single copy event hemizygous transgene for the transgene insert

TABLE 5
Media recipes

| | Stock Name | Amount |
|---|---|---|
| Recovery | MS Basal Salt Mixture | 4.30 g/L |
| or Callus | Sucrose | 20.00 g/L |
| Induction | Glucose | 5.00 g/L |
| Medium | Dicamba 1 mg/ml | 5.00 ml |
| (“CIM”) | 2,4-D 1 mg/ml | 0.10 ml |
| | Gelzan | 2.50 g/L |
| | G5 Additions 100× | 10.00 ml |
| | Silver Nitrate10 mg/ml | 1.00 ml |
| | Timentin 100 mg/ml | 2.00 ml |
| Selection | MS Basal Salt Mixture | 4.30 g/L |
| 1 medium | Proline [C5H9NO2] | 1.38 g/L |
| (if PMI | Casein Hydrolysate Enzymatic | 0.10 g/L |
| used) | Asparagine [C4H8N2O3] | 0.79 g/L |
| | Sucrose [C12H22O11] | 5.00 g/L |
| | Dicamba 1 mg/ml | 5.00 ml |
| | Mannose | 10.00 g/L |
| | Gelzan | 2.50 g/L |
| | MC15a Vitamins 1000× | 1.00 ml |
| | Timentin 100 mg/ml | 2.00 ml |
| | Silver Nitrate 10 mg/ml | 1.00 ml |
| Selection | JMS Salt Mix | 4.30 g/L |
| 2 medium | Sucrose [C12H22O11] | 5.00 g/L |
| (if PMI | Dicamba 1 mg/ml | 5.00 ml |
| used) | Mannose | 15.00 g/L |
| | G5 Additions 100× | 10.00 ml |
| | Gelzan | 2.50 g/L |
| | Silver Nitrate 10 mg/ml | 1.00 ml |
| | Timentin 100 mg/ml | 2.00 ml |
| Regeneration | MS Basal Salt Mixture | 4.3 g/L |
| medium | MS Vitamins 1000× | 1 ml |
| | Sucrose | 20 g/L |
| | Ancymidol | 1 ml |
| | Potassium Phosphate, Monobasic [KH2PO4] | 0.17 g/L |
| | CuSO4*5H2O 5 mg/ml | 7 ml |
| | Mannose | 7 g/L |
| | Gelzan | 2.4 g/L |
| | Kinetin 1 mg/ml | 1 ml |
| | Timentin 100 mg/ml | 2 ml |
| | TDZ 1 mg/ml | 0.2 ml |
| | IAA 1 mg/ml | 0.5 ml |
| Rooting | MS Basal Salt Mixture | 4.3 g/L |
| medium | MS Vitamins 1000× | 1.0 ml |
| | Sucrose | 30.0 g/L |
| | Gelzan | 2.4 g/L |
| | PPM | 5.0 ml |
| | Timentin 100 mg/ml | 2.0 ml |
| | NAA 1 mg/ml | 0.50 ml |
| | IAA 1 mg/ml | 0.25 ml |
| Co-cultivation | LS Modified Majors 20× | 50.0 ml/L |
| medium | LS Minors 1000× | 1 ml/L |
| (“COC”) | MS Iron 200× | 5 ml/L |
| | Proline [C5H9NO2] | 0.7 g/L |
| | Dicamba 1 mg/ml | 5 ml/L |
| | Sucrose [C12H22O11] | 20 g/L |
| | Glucose [C6H12O6] | 10 g/L |
| | MES [C6H13NO4S] | 0.5 g/L |
| | Purified Agar | 13 g/L |
| | JT Additions 100× | 10 ml/L |
| | Kinetin 1 mg/ml | 0.2 ml/L |
| | Acetosyringone 40 mg/ml | 2.5 ml/L |

### Example 4 Characterization of an Egg Cell Specific Promoter (prDsEC) from Boechera Stricta

The protein sequence of DD45/EC1.2 (At2g21740) was used to blast the genome sequence of Boechera stricta v1.2 genome sequence in public JGI (Joint Genome Institute) database Phytozyme 11 using blastp. Bostr.5022s0054.1 was identified as the orthologue of DD45/EC1.2 with 93% identify at amino acid sequence level and named as BsDD45. The 2 kb promoter and 5′ UTR of Bs45 was retrieved from Boechera stricta genome sequence, and 992 bp were selected to serve as prDsEC identified as SEQ ID NO. 228. To test expression a vector was constructed containing prDsEC, ZsGreen, tNOS, prUBI1, cPMI, and tUbI1 (construct 25055 in Table 3). Fluorescent microscopy is used to confirm egg-cell expression of ZsGreen fluorescent protein driven by the prDsEc promoter.

### Example 5 Enhancement of Synthetic Apomixis with Egg Cell Specific Expression of BdBBM and SiBBM Genes

BdWUS, BdBBM1 and SiBBM2 genes are also expressed under the control of egg-specific promoter (prDsEC) to test their effect on enhancing apomixis in a triple knockout of meiotic genes REC8, PAIR1, and OSD1 plant background that turns the process of meiosis to that of mitosis (Mitosis instead of Meiosis or MiMe) and results in unreduced gametes (2N instead of 1N) (Mieulet D, et al, 2016, Cell Res 26: 1242-1254). Use of MiMe background and expression of OsBBM1 under the control of Arabidopsis egg cell-specific promoter prAtDD45 for engineering of synthetic apomixis has been described in Khanday et al. Nature, Vol. 565, Jan. 3, 2019 which is incorporated by reference. MiMe background can be generated by targeted mutagenesis of REC8, PAIR1, and OSD1 genes through the use of site-specific nucleases such as CRISPR-Cas systems (Jaganathan et al, 2018, Front. Plant Sci., 17 Jul. 2018 I or targeted suppression of these genes through RNAi-mediated silencing (Rajeevkumar et al, 2015 Front. Plant Sci., 10 Sep. 2015|).

### REFERENCES

Gordon-Kamm B et al, 2019, Using Morphogenic Genes to Improve Recovery and Regeneration of Transgenic Plants. Plants 8:38, doi:10.3390/plants8020038
Jaganathan D, et al, 2018, CRISPR for Crop Improvement: An Update Review. Front. Plant Sci. doi.org/10.3389/fpls.2018.00985
Khanday I, Skinner D, Yang B, Mercier R, Sundaresan V (2019) A male—expressed rice embryogenic trigger redirected for asexual propagation through seeds. Nature 565: 91-95
Lowe K, et al. (2016) Morphogenic Regulators Baby boom and Wuschel Improve Monocot Transformation. Plant Cell 28, 1998-2015.
Mieulet D, et al, 2016, Turning rice meiosis into mitosis. Cell Res 26: 1242-1254
Negrotto, D., et al. (2000). The use of phosphomannose-isomerase as a selectable marker to recover transgenic maize plants (Zea mays L.) via Agrobacterium transformation. Plant Cell Rep. 19, 798-803. doi: 10.1007/s002999900187
Que, Q., and Nicholl, D. (2012). Enhanced Transformation of Recalcitrant Monocots. United States Patent Application Publication. US2012/0278950 A1.
Que, Q., et al. (2014). Maize transformation technology development for commercial event generation. Frontiers in Plant Science 5.
Rajeevkumar S, et al, 2015, Epigenetic silencing in transgenic plants. Front. Plant Sci., doi.org/10.3389/fpls.2015.00693
Zhong, H., et al. (2018). Advances in Agrobacterium-mediated Maize Transformation. In: Maize: Methods and Protocols—Lagrimini, L. M., ed. New York, N.Y.: Springer New York. 41-59.

1. A method for improving transformation efficiency of a plant, comprising transforming a plant with a nucleic acid encoding the amino acid sequence is selected from the group consisting of SEQ ID NO: 143 and SEQ ID NO: 123 or a nucleic acid encoding a polypeptide comprising an amino acid sequence having an at least 85% identity with the amino acid sequence selected from the group consisting of SEQ ID NO: 143 and SEQ ID NO: 123 and having an effect that improves transformation efficiency of a plant.

2. A method for improving transformation efficiency of a plant, comprising overexpressing the amino acid sequence selected from the group consisting of SEQ ID NO: 143 and SEQ ID NO: 123 or an amino acid sequence having an at least 85% identity with the amino acid sequence selected from the group consisting of SEQ ID NO: 143 and SEQ ID NO: 123, wherein transformation efficiency of the plant is improved.

3. The method according to claim 1, wherein the plant is a monocotyledon.

4. The method according to claim 3, wherein the monocotyledon is selected from the group consisting of corn, wheat, barley, rice, sorghum, and rye.

5. The method according to claim 1, wherein the plant is a dicotyledon.

6. The method according to claim 5, wherein the dicotyledon is selected from the group consisting of soybean, sunflower, watermelon, or Arabidopsis.

7. The method according to claim 1, wherein the improvement of transformation efficiency of a plant comprises one or more of:
a. improvement of efficiency of callus formation of the plant;
b. improvement of redifferentiation rate of the plant; and
c. improvement of gene transfer efficiency.

8. A nucleic acid construct comprising:
a. a nucleic acid encoding the amino acid sequence selected from the group consisting of SEQ ID NO: 143 and SEQ ID NO: 123 or a nucleic acid encoding a polypeptide comprising an amino acid sequence having at least 85% identity with the amino acid sequence selected from the group consisting of SEQ ID NO: 143 and SEQ ID NO: 123 and having an effect that improves transformation efficiency of a plant; and
b. a promoter for producing a nucleic acid in the plant.

9. The nucleic acid construct according to claim 8, wherein the promoter is a constitutive promoter, an inducible promoter, or a tissue-specific promoter.

10. A method of transforming a plant, comprising introducing into a plant a nucleic acid construct according to claim 8, further comprising a second nucleic acid to be expressed in the plant.

11. The method of transformation according to claim 10, wherein the transformation is transient.

12. The method of transformation according to claim 10, wherein the transformation is stable.

13. A transformed plant obtained by the method of transformation according to claim 10.

14. A nucleic acid construct comprising:
a. a nucleic acid encoding the amino acid sequence selected from the group consisting of SEQ ID NO: 143 and SEQ ID NO: 123 or a nucleic acid encoding a polypeptide comprising an amino acid sequence having at least 85% identity with the amino acid sequence selected from the group consisting of SEQ ID NO: 143 and SEQ ID NO: 123; and
b. a promoter for producing the nucleic acid in a plant.

15. The nucleic acid construct according to claim 14, further comprising a desired nucleic acid to be produced in the plant.

16. A method for improving transformation efficiency of a plant, comprising transforming a plant with
a. a nucleic acid encoding the amino acid sequence selected from the group consisting of SEQ ID NO: 143 and SEQ ID NO: 123 or a nucleic acid encoding a polypeptide comprising an amino acid sequence having an at least 85% identity with the amino acid sequence selected from the group consisting of SEQ ID NO: 143 and SEQ ID NO: 123; and
b. a nucleic acid encoding a BABY BOOM amino acid sequence;
wherein the transformation efficiency of a plant is improved compared to a wildtype plant.

17. The method of claim 16, wherein the nucleic acid encoding a BABY BOOM amino acid sequence is selected from the group consisting of SEQ ID NO: 179, SEQ ID NO: 180, and SEQ ID NO: 181.

18. A nucleic acid construct comprising:
a. a nucleic acid encoding the amino acid sequence selected from the group consisting of SEQ ID NO: 143 and SEQ ID NO: 123 or a nucleic acid encoding a polypeptide comprising an amino acid sequence having at least 85% identity with the amino acid sequence selected from the group consisting of SEQ ID NO: 143 and SEQ ID NO: 123;
b. a nucleic acid encoding a BABY BOOM amino acid sequence; and
c. a promoter for producing the nucleic acid of a. and b. in a plant.

19. The nucleic acid construct according to claim 18, further comprising a desired nucleic acid to be produced in the plant.

20. The nucleic acid construct according to claim 18, wherein the nucleic acid encoding a BABY BOOM amino acid sequence is selected from the group consisting of SEQ ID NO: 179, SEQ ID NO: 180, and SEQ ID NO: 181.

21. A nucleic acid construct comprising a sequence selected from the group consisting of SEQ ID NO: 179, SEQ ID NO: 180, and SEQ ID NO: 181 operably linked to a heterologous regulatory sequence.

22. A method of increasing the transformation efficiency of a plant, comprising transforming a plant with a nucleic acid set forth in SEQ ID NO: 179 or a nucleic acid sequence having an at least 85% identity with the sequence set forth in SEQ ID NO: 179; wherein the transformation efficiency of a plant is improved compared to a wildtype plant.

23. A method for producing a haploid plant comprising
a. transforming a plant cell with a nucleic acid encoding the amino acid sequence selected from the group consisting of SEQ ID NO: 205 and SEQ ID NO: 211 under the control of a promoter to produce a transgenic plant cell, wherein the promoter is selected from the group consisting of a haploid tissue specific promoter, an inducible promoter and a promoter that is both haploid-tissue specific and inducible;
b. generating a transgenic plant from said transgenic plant cell;
c. overexpressing the nucleic acid encoding the amino acid sequence selected from the group consisting of SEQ ID NO: 205 and SEQ ID NO:
211 in a haploid tissue of said transgenic plant to produce a haploid somatic embryo; and
d. growing said embryo into a haploid plant.

24. The method according to claim 23, wherein the promoter is an egg-cell preferred promoter.

25. The method according to claim 23, wherein the promoter is SEQ ID NO. 288.

26. The method according to claim 23, wherein the plant is a monocotyledon.

27. The method according to claim 26, wherein the monocotyledon is corn.

28. The method according to claim 23, wherein the plant comprises the matrilineal haploid induction locus.

29. A haploid plant obtained by the method of claim 23.

30. A recombinant DNA molecule comprising a DNA sequence selected from the group consisting of:
a) a sequence with at least 85 percent sequence identity to SEQ ID NO:288;
b) a fragment of SEQ ID NO:288, wherein the fragment has gene-regulatory activity;
wherein said DNA sequence is operably linked to a heterologous transcribable DNA molecule.

31. A method of propagating from one or more gametophytic or sporophytic cells in an ovule of a plant in the absence of egg cell fertilization, the method comprising:
transforming a plant with a gene construct comprising a nucleic acid encoding a polypeptide having at least 95% sequence identity to the polypeptide sequence selected from the group consisting of SEQ ID NO: 205 and SEQ ID NO: 211, wherein the nucleic acid is operably linked to a promoter; and
growing and selecting a progeny plant from the one or more gametophytic or sporophytic cells, wherein the progeny plant contains one or more sets of chromosomes from the transformed plant, and wherein propagation of the plant occurs in the absence of egg cell fertilization.

32. The method according to claim 31, wherein the plant comprises the matrilineal haploid induction locus.

33. The method according to claim 31, wherein the plant comprises modifications to alter meiosis to mitosis.

34. The method according to claim 33, wherein the plant comprises knockouts of the meiotic genes REC8, PAIR1, and OSD1.

35. The method according to claim 31, wherein the promoter is an egg-cell preferred promoter.

36. The method according to claim 35, wherein the promoter is SEQ ID NO. 288.

37. The method according to claim 31, wherein the plant is a monocotyledon.

38. The method according to claim 37, wherein the monocotyledon is corn.

39. A plant produced by the method of claim 31.
